(19) United States
(12) Patent Application Publication
(10) Pub. No.: US 2025/0207109 A1
(43) Pub. Date: Jun. 26, 2025

(54) ENGINEERED SPLIT DHFR-BASED METHODS AND SYSTEMS FOR SELECTING CELLS THAT HAVE STABLY ACQUIRED A HETEROLOGOUS POLYNUCLEOTIDE

(71) Applicant: DNA Twopointo, Inc., Newark, CA (US)

(72) Inventors: Nigel Killeen, Hillsborough, CA (US); Renee Saville, Mountain View, CA (US); Jeremy Minshull, Los Altos, CA (US); Sridhar Govindarajan, Los Altos, CA (US)

(73) Assignee: DNA Twopointo, Inc., Newark, CA (US)

(21) Appl. No.: 18/709,446

(22) PCT Filed: Sep. 20, 2023
(86) PCT No.: PCT/US23/74680

§ 371 Date: May 10, 2024

Related U.S. Application Data
(60) Provisional application No. 63/376,399, filed on Sep. 20, 2022.

(51) Int. Cl. C12N 9/06; C07K 14/39; C12Q 1/26; G01N 21/64

(52) U.S. Cl. CPC C12N 9/003; C07K 14/39; C12Q 1/26; C12Y 105/01003; G01N 21/6428; C07K 2319/00; G01N 2021/6439

(57) ABSTRACT

Engineered split dihydrofolate reductase-based methods and systems are provided for selecting cells that have stably acquired a heterologous polynucleotide.

### CROSS-REFERENCE TO RELATED APPLICATIONS

This application claims priority from U.S. Provisional Patent Application No. 63/376,399, filed on Sep. 20, 2022, which is incorporated by reference herein in its entirety.

### SEQUENCE LISTING

A Sequence Listing has been submitted electronically in XML format and is hereby incorporated by reference in its entirety. The XML copy, created on Sep. 20, 2023, is named SDHFR_ST26.xml and is 287,000 bytes in size.

### BACKGROUND

Transferring genetic material into cells is fundamental to contemporary forms of adoptive cell therapy. In many cases, such transfers involve vectors derived from viruses (such as retroviruses or adeno-associated virus). Alternatively, the transfers involve non-viral delivery procedures (such as electroporation) that provide cells with nucleoprotein complexes, DNA or mRNA molecules, or combinations thereof.

In their most straightforward form, gene transfers accomplish simple gain-of-function effects, conferring upon cells the capacity to express novel proteins and/or variant forms of endogenous proteins, with the acquired proteins providing some form of therapeutic benefit on the treated cells. Rendering T lymphocytes capable of expressing a chimeric antigen receptor (i.e., the process of generating CAR-T cells) is an example of this kind of gain-of-function effect. The acquired chimeric antigen receptor on such T cells gives them a means for distinguishing pathogenic cells (most typically, tumor cells) from normal cells and causing the pathogenic cells to be eliminated.

Gene transfers can also be exploited to accomplish loss-of-function effects. In the context of adoptive cell therapy, such effects can provide a range of benefits, either by way of directly enhancing therapeutic efficacy (e.g., by causing cells to differentiate in an appropriate manner) or by enhancing their capacity to survive in the adoptive host (e.g., by inducing proliferation, counteracting apoptosis, or compromising the capacity of the host to cause their rejection). The inactivation of genes that promote T cell exhaustion or allow for recognition by host immune cells are among various loss-of-function effects being explored for their benefit in improving CAR-T cell and other T cell therapies.

Advanced forms of adoptive cell therapies may be created by gene transfer processes that combine multiple genetic effects to accomplish a plurality of beneficial outcomes. Some of these effects may control how the cells recognize pathology, others the specific kinds of responses the cells make after such recognition. Still further effects may influence the capacity of the cells to migrate to particular locations in the body, their ability to avoid undesirable phenotypes (e.g., exhaustion), and their ability to acquire beneficial phenotypes for the long-term. Finally, safety mechanisms (embodied in transgenes) will be required to ensure that there are multiple ways to control or eliminate the cells should they prove harmful.

Methodology that will facilitate complex forms of gene transfer in primary cells is of considerable interest and potential impact. The transfer of large DNA molecules to cells with high efficiency is obviously one kind of advance that will allow multiple transgenes to be delivered to cells, and thus provide a means for increasing the complexity of genetic effects. An alternative—but also complementary—advance will depend on improved selection or cell sorting procedures that permit the facile enrichment of cells that have undergone gene transfer successfully.

In the research setting, scientists routinely employ a plurality of drug resistance genes as the basis for selecting cells that have stably acquired more than one kind of exogenously provided DNA molecule. Many of these drug resistance genes are of prokaryotic origin, rendering them largely unsuitable for use in therapeutic cells because of immunogenicity. It is of interest, therefore, to develop drug selection systems that are based on human proteins and, thus, should be largely free of immunogenicity concerns.

Methotrexate is an antifolate drug that competitively inhibits the human enzyme dihydrofolate reductase (DHFR). DHFR is responsible for converting dihydrofolate into tetrahydrofolate in cells. Tetrahydrofolate is essential for the de novo synthesis of nucleic acid precursors that include thymidilic acid. Because a deficiency of DHFR activity compromises cell growth and proliferation, methotrexate has proven useful in treating certain kinds of cancers.

The human DHFR enzyme may be mutated such that it demonstrates resistance to otherwise toxic concentrations of methotrexate. A DHFR mutein carrying both Phenylalanine in place of Leucine-22 and Serine in place of Phenylalanine-31 (i.e., DHFR-L22F/F31S, or DHFR^{FS}) is an example of one such methotrexate-resistant form of DHFR. Gene transfer with a vector encoding DHFR^{FS} allows for the survival of cells in concentrations of methotrexate that kill non-transduced/non-transfected cells. Thus, DHFR^{FS} can be exploited as the basis of a drug selection system in gene transfer situations. Importantly, because the mutein is of human origin, minimal concerns about immunogenicity are limited to those that relate solely to the two amino acid substitutions used (i.e., L22F and F31S).

A fully functional form of murine DHFR can be generated by expressing two fragments of the enzyme (i.e., “split DHFR”) in cells in such a manner that they associate to reconstitute enzymatic activity. The fragments comprise pieces of the protein sequence that are normally contiguous with one another, their breakpoint occurring in a surface exposed loop containing residues 101-108. Reconstitution requires that the fragments are physically proximal to one another inside the cell, as is the case if they are fused to protein moieties that have a capacity to form stable dimers. For example, fusions to the homo-dimerizing GCN4 leucine zipper polypeptide are an effective means for accomplishing the required stable association. If the DHFR fragments derive from a methotrexate-resistant mutein (such as DHFR^{FS}), then methotrexate can be used to select for cells that carry the two fragments.

Split DHFR has been used as a screening assay for protein-protein interactions. It has not, however, been used routinely as a means for selecting cells that have undergone gene transfer successfully with two different DNA molecules. Moreover, while there has been success using the mouse form of DHFR in a split context, the human DHFR protein is not functional when it is split in a similar fashion to the mouse protein. Although the mouse and human orthologous proteins are highly similar to one another, they differ at nineteen of one hundred and eighty-seven residues. While these differences must account for why human DHFR loses activity when split, they also attach a risk of immunogenicity to the mouse protein if it is expressed in humans.

For split DHFR to be used as a selection system in therapeutic human cells, there is a need to solve both of the issues just mentioned, i.e., to engineer the human protein such that it retains activity when split and to do so with reduced immunogenicity risk relative to the fully mouse version of the split enzyme.

### SUMMARY

In one aspect, a method is provided for selecting cells that have stably acquired a heterologous polynucleotide, the method comprising: (A) co-transfecting a plurality of cells with: (1) a first heterologous polynucleotide comprising a nucleotide sequence that encodes a first fusion protein, the first fusion protein comprising: (a) a first dimerization domain; and (b) a first DHFR fragment; and (2) a second heterologous polynucleotide comprising a nucleotide sequence that encodes a second fusion protein, the second fusion protein comprising: (a) a second dimerization domain; and (b) a second DHFR fragment that is normally contiguous to the first DHFR fragment, wherein the fragments of the DHFR protein sequence are catalytically inactive in isolation or when co-expressed in cells, but when brought into proximity with one another by fusion to protein domains that co-associate, confer resistance to methotrexate; and (B) subjecting the cells to methotrexate.

In one aspect, a system is provided for selecting cells that have stably acquired a heterologous polynucleotide, the system comprising: (1) a first heterologous polynucleotide comprising a nucleotide sequence that encodes a first fusion protein, the first fusion protein comprising: (a) a first dimerization domain; and (b) a first DHFR fragment; and (2) a second heterologous polynucleotide comprising a nucleotide sequence that encodes a second fusion protein, the second fusion protein comprising: (a) a second dimerization domain; and (b) a second DHFR fragment that is normally contiguous to the first DHFR fragment, wherein the fragments of the DHFR protein sequence are catalytically inactive in isolation or when co-expressed in cells, but when brought into proximity with one another by fusion to protein domains that co-associate, confer resistance to methotrexate.

In one aspect, a modified cell is provided, the modified cell expressing: (1) a gene of interest; (2) a first heterologous polynucleotide comprising a nucleotide sequence that encodes a first fusion protein, the first fusion protein comprising: (a) a first dimerization domain; and (b) a first DHFR fragment; and (3) a second heterologous polynucleotide comprising a nucleotide sequence that encodes a second fusion protein, the second fusion protein comprising: (a) a second dimerization domain; and (b) a second DHFR fragment that is normally contiguous to the first DHFR fragment, wherein the fragments of the DHFR protein sequence are catalytically inactive in isolation or when co-expressed in cells, but when brought into proximity with one another by fusion to protein domains that co-associate, confer resistance to methotrexate.

In another aspect, a method is provided for determining rimiducid-dependent dimerization of FK506 binding proteins (FKBPs), the method comprising: (A) co-transfecting a cell with: (1) a first heterologous polynucleotide comprising a nucleotide sequence that encodes a first fusion protein, the first fusion protein comprising: (a) a first engineered FKBP-based dimerization domain, wherein the first engineered FKBP-based dimerization domain has the ability to be activated upon binding of a chemical inducer of dimerization (CID); and (b) a first DHFR fragment, and (2) a second heterologous polynucleotide comprising a nucleotide sequence that encodes a second fusion protein, the second fusion protein comprising: (a) a second engineered FKBP-based dimerization domain; and (b) a second DHFR fragment that is normally contiguous to the first DHFR fragment, wherein the fragments of the DHFR protein sequence are catalytically inactive in isolation or when co-expressed in cells, but when brought into proximity with one another by fusion to protein domains that co-associate, demonstrate resistance to methotrexate; and (B) subjecting the cell to: (1) the CID; and (2) methotrexate.

### BRIEF DESCRIPTION OF THE FIGURES

The accompanying figures, which are incorporated in and constitute a part of the specification, are used merely to illustrate various example embodiments.

FIG. 1 shows the results of flow cytometric analysis of Jurkat T lymphoma cells that were transfected with pairs of transposon constructs encoding DHFR fragments subject to dimerization by leucine zipper or coiled coil peptides as indicated. Successful methotrexate selection of cells carrying both transfected transposons was evident by the fact that the majority of cells expressed both mTagBFP2 and plobRFP. A transgene encoding one or the other of these fluorescent proteins was present on the transposons used in each case.

FIG. 2 provides an alignment of the human and mouse DHFR protein sequences (involving NCBI reference sequences NP_000782.1 and NP_034179.1, respectively). The protein sequences are shown without the initiator methionine residues, and the numbering convention used throughout this document reflects this elision. Amino acids that differ between the two species are highlighted. Leucine-22 and Phenylalanine-31 are shown in bold, these being the residues that are mutated to Phenylalanine and Serine, respectively, to create a methotrexate-resistant form of the enzyme (DHFR^{FS}). An unnatural Leucine substitution for Phenylalanine-179 (from MacDonald C and Piper R C. Puromycin and Methotrexate Resistance Cassettes and Optimized cre-recombinase Expression Plasmids for use in Yeast. Yeast. 2015; 32 (5): 423-438) was used in carboxy-terminal fragments of human DHFR (identified with an asterisk in FIGS. 3, 6, 11, and 15). Reversion of this substitution to Phenylalanine improved resistance to methotrexate as shown below.

FIG. 3 depicts chimeric carboxy-terminal fragments of DHFR that differ from one another in their relative content of mouse or human orthologous substitutions. The numbers at the top of the diagram correspond to the differing residues (as explained above in the description of FIG. 2, the asterisk identifies a non-natural difference at position 179; the starting human sequence contained a Leucine at this position, whereas the starting mouse sequence contained Phenylalanine, which is normally invariant at this position for both species). The carboxy-terminal fragments initiated at a pair of Leucine residues in place of Leucine 105. These fragments were expressed from transgenes that provided amino-terminal heterodimerizing coiled coil peptide sequences (P7A and P8A for the amino-terminal and carboxy-terminal fragments, respectively). The transgene encoding the amino-terminal fragment was present in a transposon that also carried a transgene expressing mTagBFP2, while the carboxy-terminal fragment transgene was instead paired with a plobRFP transgene.

FIG. 4 shows flow cytometry data collected from cells that had been co-transfected with transposons expressing the carboxy-terminal fragments depicted in FIG. 3 in each case together with a transposon expressing an amino-terminal fragment of the enzyme that was entirely mouse in its protein sequence (except for the Phenylalanine and Serine substitutions at positions 22 and 31, respectively, which are required for DHFR to confer resistance to methotrexate). The transfected cells were selected in 200 nM methotrexate for one week prior to analysis. The bivariate plots show relative BFP fluorescence on the X-axis and RFP fluorescence on the Y-axis (from mTagBFP2 and plobRFP, respectively). In the experiment represented in FIG. 4, and all the similar experiments in the following figures, the BFP reporter was expressed from the transposon carrying the transgene for the amino-terminal fragment of DHFR, while the RFP reporter was expressed from the transposon carrying the transgene encoding the carboxy-terminal fragment of DHFR. The identity of the carboxy-terminal fragment variant no. used in each case is shown in the bottom left-hand corner of the bivariate plots. The numbers at top center of each plot provide the percentages of BFP+RFP+ cells (i.e., those cells found in the rectangular gate at the top right of the plots).

FIG. 5 shows bivariate plots of the mTagBFP2 and plobRFP geometric mean fluorescence intensities (MFIs) measured for cells transfected with the collection of carboxy-terminal DHFR fragments shown in FIG. 3 combined with a fully mouse amino-terminal fragment. The MFIs were derived from the data shown in FIG. 4. The sizes of the data points vary as a function of the percentages of cells that were BFP+RFP+ in each case. The labels next to each data point correspond to the carboxy-terminal variant number as depicted in FIG. 3.

FIG. 6 depicts chimeric DHFR fragments that differ from one another in their relative content of mouse or human orthologous substitutions. The amino-terminal fragments (terminating at Leucine-105) are shown on the left, and the carboxy-terminal fragments (from Leucine-105, as explained with respect to FIG. 3) are shown on the right. These fragments were expressed from transgenes that provided amino-terminal heterodimerizing coiled coil peptide sequences (P7A and P8A). The transgene encoding the amino-terminal fragment was present in a transposon that also carried a transgene expressing mTagBFP2, while the carboxy-terminal fragment transgene was instead paired with a plobRFP transgene. The numbers at the top of the two diagrams correspond to the residues that differ between mouse and human DHFR. As an example, variant no. 13 has a largely human protein sequence except for residues 2 and 3, which were changed to the amino acids present in the mouse protein.

FIGS. 7A-7C show flow cytometry data generated by co-transfecting Jurkat cells with transposons encoding various amino-terminal and carboxy-terminal variants of DHFR as depicted in FIGS. 6, 11, and 15. The cells were selected in methotrexate (200 nM) for a week before analysis. FIG. 7A shows mTagBFP2 fluorescence (X-axis) by plobRFP fluorescence (RFP; Y-axis) for 40 amino-terminal variants (selected from the collections depicted in FIGS. 6 and 11; the relevant variant no. is provided in each bivariate plot) combined with carboxy-terminal fragment variant no. 46 or “Version 46.” Similarly, FIGS. 7B and 7C show the results obtained with the amino terminal variant collection combined with carboxy terminal variant nos. 48 and 53, respectively. The percentage of cells falling in the BFP+RFP+ rectangular gate at the top right is shown in each case. The control amino-terminal fragment was entirely mouse in protein sequence (with the exception of the L22F and F31S substitutions required to create a methotrexate-resistant form of DHFR).

FIGS. 8A-8C show bivariate plots of the mTagBFP2 and plobRFP geometric MFIs measured for cells transfected with transposons encoding the collection of amino-terminal variants shown in FIG. 6 combined with transposons encoding the carboxy-terminal variant dubbed “Version 46.” The full collection of amino-terminal variants combined with carboxy-terminal Version 46 is represented in FIG. 8A (and summarizing all the data in FIG. 7A), while FIGS. 8B and 8C show the indicated portions of the distribution in the two rectangles shown in FIG. 8A. The viability of the cultures on the day of analysis (as assessed by flow cytometric light scatter) is included in FIGS. 8B and 8C. Good DHFR activity is associated with low MFIs for both fluorescent proteins and high cell viability. The best performing amino-terminal variant is highlighted with an arrow (FIG. 8C).

FIGS. 9A-9C show bivariate plots of the mTagBFP2 and plobRFP geometric MFIs measured for cells transfected with transposons encoding the collection of amino-terminal variants shown in FIG. 6 combined with transposons encoding the carboxy-terminal variant dubbed variant no. 48 or “Version 48.” The full collection of amino-terminal variants combined with carboxy-terminal Version 48 is represented in FIG. 9A (and summarizing all the data in FIG. 7B), while FIGS. 9B and 9C show the indicated portions of the distribution in the two rectangles shown in FIG. 9A. The viability of the cultures on the day of analysis (as assessed by flow cytometric light scatter) is included in FIGS. 9B and 9C. Good DHFR activity is associated with low MFIs for both fluorescent proteins and high cell viability. The best performing amino-terminal variant is highlighted with an arrow (FIG. 9C).

FIGS. 10A-10C show bivariate plots of the mTagBFP2 and plobRFP geometric MFIs measured for cells transfected with transposons encoding the collection of amino-terminal variants shown in FIG. 6 combined with transposons encoding the carboxy-terminal variant dubbed variant no. 53 or “Version 53.” The full collection of amino-terminal variants combined with carboxy-terminal Version 53 is represented in FIG. 10A (and summarizing all the data in FIG. 7C), while FIGS. 10B and 10C show the indicated portions of the distribution in the two rectangles shown in FIG. 10A. The viability of the cultures on the day of analysis (as assessed by flow cytometric light scatter) is included in FIGS. 10B and 10C. Good DHFR activity is associated with low MFIs for both fluorescent proteins and high cell viability. The best performing amino-terminal variant is highlighted with an arrow (FIG. 10C).

FIG. 11 depicts chimeric DHFR fragments in which a single human substitution is present in an otherwise entirely mouse context.

FIG. 12 shows a bivariate plot of the mTagBFP2 and plobRFP geometric MFIs obtained in cells transfected with transposons encoding the collection of amino-terminal variants shown in FIG. 11 combined with transposons encoding the Version 46 carboxy-terminal fragment. The data point labels correspond to the residues that were changed in each case. Cell viability on the day of analysis, as assessed flow cytometrically using forward and orthogonal light scatter, is reflected in the relative sizes of the data points, as indicated in the legend.

FIG. 13 shows a bivariate plot of the mTagBFP2 and plobRFP geometric MFIs obtained in cells transfected with transposons encoding the collection of amino-terminal variants shown in FIG. 11 combined with transposons encoding the Version 48 carboxy-terminal fragment. The data point labels correspond to the residues that were changed in each case. Cell viability on the day of analysis, as assessed flow cytometrically using forward and orthogonal light scatter, is reflected in the relative sizes of the data points, as indicated in the legend.

FIG. 14 shows a bivariate plot of the mTagBFP2 and plobRFP geometric MFIs obtained in cells transfected with transposons encoding the collection of amino-terminal variants shown in FIG. 11 combined with transposons encoding the Version 53 carboxy-terminal fragment. The data point labels correspond to the residues that were changed in each case. Cell viability on the day of analysis, as assessed flow cytometrically using forward and orthogonal light scatter, is reflected in the relative sizes of the data points, as indicated in the legend.

FIG. 15 depicts a collection of variants that were used to determine a minimal set of mouse amino acid substitutions that would confer good activity on a split form of human DHFR.

FIGS. 16A-16C show bivariate plots of the mTagBFP2 and plobRFP geometric MFIs measured for cells transfected with transposons encoding the six amino-terminal variants shown in FIG. 15 combined with transposons encoding the six carboxy-terminal variants also shown in FIG. 15. The full collection of amino-terminal variants is represented in FIG. 16A, while FIGS. 16B and 16C show the indicated portions of the distribution in FIG. 16A. The viability of the cultures on the day of analysis (as assessed by flow cytometric light scatter) is included in the lower graphs. Four control transfections were included in this experiment, all involving mouse DHFR fragments dimerized with the GCN4 leucine zipper peptides (LZ), the N7/N8, or the P7A/P8A pairs of dimerizing coiled coil peptides, or an alternative version of the fragments in which the N7/N8 peptides were attached at the carboxy-termini rather than the amino-termini of the mouse DHFR fragments. The best-performing variant combinations—either of which represents a minimal content of mouse substitutions associated with optimal activity—are highlighted with arrows.

FIGS. 17A-17C depict a repeat of the experiment shown in FIGS. 16A-16C but involving just amino-terminal variant nos. 65 and 69 combined in each case with all six carboxy-terminal variants. An additional repeat experiment of the same design yielded similar results.

FIG. 18 shows use of an example human split DHFR as the basis for selecting Jurkat cells carrying two co-transfected DNA molecules collectively harboring five transgenes (not including the two split DHFR selection genes). In each of the five cases, the plasmids used were both ˜10 Kb in size. The transgenes present on the plasmids used are indicated in the table beneath the flow cytometry data. The bivariate plots show expression of the BCMA- or CD19-specific CARs and CD360 (the alpha chain of the receptor for human IL-21) on the transfected cells; the BCMA-specific CAR was detected using an Alexa-647-conjugated form of a recombinant human BCMA-Fc fusion protein (R&D Systems, Minneapolis, MN, USA) while the CD19-specific CAR was detected using a phycoerythrin-conjugated form of recombinant human CD19 (ACROBiosystems, Newark, DE, USA); CD360 was detected with a Brilliant Violet 421-conjugated monoclonal antibody specific for human CD360 (BioLegend Inc., San Diego, CA, USA). Expression of the GD2-specific CAR was not assessed.

FIG. 19 shows expression of Luciola italica luciferase in a two-fold dilution series of the indicated Jurkat cell pools. This luciferase was expressed in the cells from the co-transfected transposons under the control of a constitutive promoter; it was assayed by adding firefly luciferin to the cells (FLAR from Targeting Systems, El Cajon, CA, USA) prior to luminometry.

FIG. 20 shows expression of an NFAT-luciferase transgene in response to activation of the indicated cell pools with a two-fold dilution series of soluble anti-CD3. The relevant luciferase was from Cypridina nocticluca; it was secreted from the cells and assayed in the supernatant fluids taken from the cell cultures by luminometry using vargulin as the substrate (with the VLAR-2 reagent from Targeting Systems, El Cajon, CA, USA).

FIG. 21 shows expression of an NFAT-luciferase transgene in response to activation of the indicated cell pools by exposing the cell pools to cloned, transfected EL4 cells. The ELA cells carried transgenes allowing them to express BCMA (for use with pools 531861 and 531864), CD19 (for use with pools 531862 and 531863), or GD2 (for use with pool 531865), all in a doxycycline-responsive fashion. The graph shows NFAT-luciferase induction (normalized to pools treated with the relevant EL4 cells not treated with doxycycline in each case). NFAT-luciferase activity was assayed as in FIG. 20.

FIG. 22 is a repeat of the experiment shown in FIG. 21 (performed in parallel) but using different clones of BCMA-, CD19-, or GD2-expressing stimulator cells.

FIG. 23 shows a difference in basal NFAT-luciferase expression in five pools of cells as a presumptive consequence of variation in CAR-dependent tonic signaling. The GD2-specific CAR expressed in the 531865 cell pool is known to be associated with a high level of such tonic signaling, whereas the CD19-specific CAR used in the 531862 and 531863 cell pools do not promote tonic signaling. The BCMA-specific CAR used in 531861 and 531864 demonstrates a moderate level of tonic signaling.

FIG. 24 shows expression of a STAT3-luciferase transgene in the CD19-specific cells stimulated as in FIG. 21. The relevant luciferase used here was from Gaussia princeps and was also secreted from the cells; it was assayed in the cell culture supernatant fluid by luminometry using coelenterazine as the substrate (and the GAR reagent from Targeting Systems of El Cajon, CA, USA).

FIG. 25 shows the functionality of an example split DHFR system in primary human T cells. The cell pools shown were generated by co-transfecting activated primary T cells (using the MaxCyte ATx electroporator) with two plasmids in both cases: one ˜ 10 Kb and the other ˜6 Kb in size, prior to selection in methotrexate (200 nM) for 3 weeks before analysis by flow cytometry. One of the plasmids used to generate pool #1 carried a transgene encoding a BCMA-specific CAR, while a transgene encoding a CD19-specific CAR was used to generate pool #2. CD360 was also (weakly) expressed from a linked transgene in each case.

FIG. 26 illustrates the results of a split DHFR complementation assay to show rimiducid-dependent dimerization of a fusion protein based on an FKBP having a molecular weight of approximately 12.6 kDa (FKBP12).

### DETAILED DESCRIPTION

### I. Definitions

Use of the singular forms “a,” “an,” and “the” include plural references unless the context clearly dictates otherwise. For example, reference to “a polynucleotide” may include a plurality of polynucleotides.

Terms such as “connected,” “attached,” “linked,” and “conjugated” are used interchangeably herein and encompass direct as well as indirect connection, attachment, linkage, or conjugation unless the context clearly dictates otherwise. Where a range of values is recited, each intervening integer value, and each fraction thereof, between the recited upper and lower limits of that range is also specifically disclosed, along with each subrange between such values. The upper and lower limits of any range can independently be included in or excluded from the range, and each range where either, neither, or both limits are included is also encompassed. Where a value being discussed has inherent limits, for example where a component can be present at a concentration of from 0 to 100%, or where the pH of an aqueous solution can range from 1 to 14, those inherent limits are specifically disclosed. Where a value is explicitly recited, values that are “about” (that is, within ±10%) the same quantity or amount as the recited value are also within the scope. Where a combination is disclosed, each sub-combination of the elements of that combination is also specifically disclosed. Conversely, where different elements or groups of elements are individually disclosed, combinations thereof are also disclosed. Where any element is disclosed as having a plurality of alternatives, examples in which each alternative is excluded singly or in any combination with the other alternatives are also hereby disclosed; more than one element can have such exclusions, and all combinations of elements having such exclusions are hereby disclosed.

Unless defined otherwise herein, all technical and scientific terms have the same meaning as commonly understood by one of ordinary skill in the relevant art. Singleton, et al., Dictionary of Microbiology and Molecular Biology, 2^{nd} Ed., John Wiley and Sons, New York (1994), and Hale & Marham, The Harper Collins Dictionary of Biology, Harper Perennial, NY, 1991, provide one of skill with a general dictionary of many of the terms used herein. Unless otherwise indicated, nucleic acids are written left to right in 5′ to 3′ orientation; amino acid sequences are written left to right in amino to carboxy orientation, respectively. The terms defined immediately below are more fully defined by reference to the specification as a whole.

The “configuration” of a polynucleotide means the functional sequence elements within the polynucleotide and the order and direction of those elements.

The terms “corresponding transposon” and “corresponding transposase” are used to indicate an activity relationship between a transposase and a transposon. A transposase transposases its corresponding transposon.

The term “coupling element” or “translational coupling element” means a DNA sequence that allows the expression of a first polypeptide to be linked to the expression of a second polypeptide. IRES elements and cis-acting hydrolase elements are examples of coupling elements.

The terms “DNA sequence,” “RNA sequence,” or “polynucleotide sequence” refer to a contiguous nucleic acid sequence. The sequence can be an oligonucleotide of 2 to 20 nucleotides in length to a full-length genomic sequence of thousands or hundreds of thousands of base pairs.

The term “expression construct” means any polynucleotide designed to transcribe an RNA, such as, for example, a construct that contains at least one promoter that is or may be operably linked to a downstream gene, coding region, or polynucleotide sequence (for example, a cDNA or genomic DNA fragment that encodes a polypeptide or protein, or an RNA effector molecule, for example, an antisense RNA, triplex-forming RNA, ribozyme, an artificially selected high affinity RNA ligand (aptamer), a double-stranded RNA, for example, an RNA molecule comprising a stem-loop or hairpin dsRNA, or a bi-finger or multi-finger dsRNA or a microRNA, or any RNA). An “expression vector” is a polynucleotide comprising a promoter that can be operably linked to a second polynucleotide. Transfection or transformation of the expression construct into a recipient cell allows the cell to express an RNA effector molecule, polypeptide, or protein encoded by the expression construct. An expression construct may be a genetically engineered plasmid, virus, recombinant virus, or an artificial chromosome derived from, for example, a bacteriophage, adenovirus, adeno-associated virus, retrovirus, lentivirus, poxvirus, or herpesvirus. Such expression vectors can include sequences from bacteria, viruses, or phages. Such vectors include chromosomal, episomal, and virus-derived vectors, for example, vectors derived from bacterial plasmids, bacteriophages, yeast episomes, yeast chromosomal elements, and viruses, vectors derived from combinations thereof, such as those derived from plasmid and bacteriophage genetic elements, cosmids, and phagemids. An expression construct can be replicated in a living cell, or it can be made synthetically. The terms “expression construct,” “expression vector,” “vector,” and “plasmid” are used interchangeably herein to demonstrate the application of the invention in a general, illustrative sense, and are not intended to limit the invention to a particular type of expression construct.

The term “expression polypeptide” means a polypeptide encoded by a gene on an expression construct.

The term “expression system” means any in vivo or in vitro biological system that is used to produce one or more gene product encoded by a polynucleotide.

A “gene transfer system” refers to a vector or gene transfer vector, i.e., a polynucleotide comprising the gene to be transferred which is cloned into a vector (a “gene transfer polynucleotide” or “gene transfer construct”). A gene transfer system may also comprise other features to facilitate the process of gene transfer. For example, a gene transfer system may comprise a vector and a lipid or viral packaging mix for enabling a first polynucleotide to enter a cell, or it may comprise a polynucleotide that includes a transposon and a second polynucleotide sequence encoding a corresponding transposase to enhance productive genomic integration of the transposon. The transposases and transposons of a gene transfer system may be on the same nucleic acid molecule or on different nucleic acid molecules. The transposase of a gene transfer system may be provided as a polynucleotide or as a polypeptide.

Two elements are “heterologous” to one another if not naturally associated. For example, a nucleic acid sequence encoding a protein linked to a heterologous promoter means a promoter other than that which naturally drives expression of the protein. A heterologous nucleic acid flanked by transposon ends or inverted terminal repeats (“ITR”s) means a heterologous nucleic acid not naturally flanked by those transposon ends or ITRs, such as a nucleic acid encoding a polypeptide other than a transposase, including an antibody heavy or light chain. A nucleic acid is heterologous to a cell if not naturally found in the cell or if naturally found in the cell but in a different location (e.g., episomal or different genomic location) than the location described.

The term “host” means any prokaryotic or eukaryotic organism that can be a recipient of a nucleic acid. A “host” includes prokaryotic or eukaryotic organisms that can be genetically engineered. For examples of such hosts, see Maniatis et al., Molecular Cloning. A Laboratory Manual, Cold Spring Harbor Laboratory, Cold Spring Harbor, N.Y. (1982). As used herein, the terms “host,” “host cell,” “host system,” and “expression host” can be used interchangeably.

An “intron” is a nucleotide sequence within a gene that is not expressed or operative in the final RNA product.

An “IRES” or “internal ribosome entry site” means a specialized sequence that directly promotes ribosome binding, independent of a cap structure.

An “isolated” polypeptide or polynucleotide means a polypeptide or polynucleotide that has been either removed from its natural environment, produced using recombinant techniques, or chemically or enzymatically synthesized. Polypeptides or polynucleotides may be purified, that is, essentially free from any other polypeptide or polynucleotide and associated cellular products or other impurities.

The terms “nucleoside” and “nucleotide” include those moieties that contain not only the known purine and pyrimidine bases, but also other heterocyclic bases that have been modified. Such modifications include methylated purines or pyrimidines, acylated purines or pyrimidines, or other heterocycles. Modified nucleosides or nucleotides can also include modifications on the sugar moiety, for example, where one or more of the hydroxyl groups are replaced with halogen, aliphatic groups, or are functionalized as ethers, amines, or the like. The term “nucleotidic unit” is intended to encompass nucleosides and nucleotides.

An “open reading frame” or “ORF” means a portion of a polynucleotide that, when translated into amino acids, contains no stop codons. The genetic code reads DNA sequences in groups of three base pairs, which means that a double-stranded DNA molecule can read in any of six possible reading frames-three in the forward direction and three in the reverse. An ORF typically also includes an initiation codon at which translation may start.

The term “operably linked” refers to functional linkage between two sequences such that one sequence modifies the behavior of the other. For example, a first polynucleotide comprising a nucleic acid expression control sequence (such as a promoter, IRES sequence, enhancer, or array of transcription factor binding sites) and a second polynucleotide are operably linked if the first polynucleotide affects transcription and/or translation of the second polynucleotide. Similarly, a first amino acid sequence comprising a secretion signal, i.e., a subcellular localization signal, and a second amino acid sequence are operably linked if the first amino acid sequence causes the second amino acid sequence to be secreted or localized to a subcellular location.

A “piggyBac-like transposase” means a transposase with at least 20% sequence identity as identified using the TBLASTN algorithm to the piggyBac transposase from Trichoplusia ni (SEQ ID NO: 79), and as more fully described in Sakar, A. et. Al., (2003). Mol. Gen. Genomics 270:173-180. “Molecular evolutionary analysis of the widespread piggyBac transposon family and related ‘domesticated’ species,” incorporated herein by reference in its entirety and further characterized by a DDE-like DDD motif, with aspartate residues at positions corresponding to D268, D346, and D447 of Trichoplusia ni piggyBac transposase on maximal alignment. PiggyBac-like transposases are also characterized by their ability to excise their transposons precisely with a high frequency. A “piggyBac-like transposon” means a transposon having transposon ends that are the same or at least 80%, including at least 90, 95, 96, 97, 98 or 99% identical to the transposon ends of a naturally occurring transposon that encodes a piggyBac-like transposase. A piggyBac-like transposon includes an ITR sequence of approximately 12-16 bases at each end. These repeats may be identical at the two ends, or the repeats at the two ends may differ at 1 or 2 or 3 or 4 positions in the two ITRs. The transposon is flanked on each side by a 4 base sequence corresponding to the integration target sequence that is duplicated on transposon integration (the “Target Site Duplication” or “Target Sequence Duplication” or “TSD”).

The terms “polynucleotide,” “oligonucleotide,” “nucleic acid,” “nucleic acid molecule,” and “gene” are used interchangeably to refer to a polymeric form of nucleotides of any length, and may comprise ribonucleotides, deoxyribonucleotides, analogs thereof, or mixtures thereof. These terms refer only to the primary structure of the molecule. Thus, the terms include triple-, double-, and single-stranded DNA, as well as triple-, double-, and single-stranded RNA. The terms also encompass modified, for example by alkylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, siRNA, and mRNA, whether spliced or unspliced, any other type of polynucleotide that is an N- or C-glycoside of a purine or pyrimidine base, and other polymers containing nonnucleotidic backbones, for example, polyamide (for example, peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration that allows for base pairing and base stacking, such as is found in DNA and RNA. There is no intended distinction in length between the terms “polynucleotide,” “oligonucleotide,” “nucleic acid,” and “nucleic acid molecule,” and these terms are used interchangeably herein. These terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (for example, methyl phosphonates, phosphotriesters, phosphoramidates, carbamates, or the like) with negatively charged linkages (for example, phosphorothioates, phosphorodithioates, or the like), and with positively charged linkages (for example, aminoalkylphosphoramidates, aminoalkylphosphotriesters), those containing pendant moieties, such as, for example, proteins (including enzymes (for example, nucleases), toxins, antibodies, signal peptides, poly-L-lysine, or the like), those with intercalators (for example, acridine, psoralen, or the like), those containing chelates (of, for example, metals, radioactive metals, boron, oxidative metals, or the like), those containing alkylators, those with modified linkages (for example, alpha anomeric nucleic acids, or the like), as well as unmodified forms of the polynucleotide or oligonucleotide.

A “promoter” means a nucleic acid sequence sufficient to direct transcription of an operably linked nucleic acid molecule. A promoter can be used together with other transcription control elements (for example, enhancers) that are sufficient to render promoter-dependent gene expression controllable in a cell type-specific, tissue-specific, or temporal-specific manner, or that are inducible by external signals or agents; such elements, may be within the 3′ region of a gene or within an intron. In one aspect, the promoter may be operably linked to a nucleic acid sequence, for example, a cDNA, a gene sequence, or an effector RNA coding sequence, in such a way as to enable expression of the nucleic acid sequence, or a promoter is provided in an expression cassette into which a selected nucleic acid sequence to be transcribed can be conveniently inserted.

The term “selectable marker” means a polynucleotide segment that allows one to select for or against a molecule or a cell that contains it, often under particular conditions. These markers can encode an activity, such as, but not limited to, production of RNA, a peptide, or a protein, or these markers can provide a binding site for RNA, peptides, proteins, inorganic and organic compounds, or compositions. Examples of selectable markers include, but are not limited to: (1) DNA segments that encode products that provide resistance against otherwise toxic compounds (e.g., antibiotics); (2) DNA segments that encode products that are otherwise lacking in the recipient cell (e.g., tRNA genes, auxotrophic markers); (3) DNA segments that encode products that suppress the activity of a gene product; (4) DNA segments that encode products that can be readily identified (e.g., phenotypic markers such as beta-galactosidase, GFP, and cell surface proteins); (5) DNA segments that bind products that are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate a desired molecule (e.g. specific protein binding sites); (9) DNA segments that encode a specific nucleotide sequence that can be otherwise non-functional (e.g., for PCR amplification of subpopulations of molecules); and/or (10) DNA segments, which when absent, directly or indirectly confer sensitivity to particular compounds.

Sequence identity can be determined by aligning sequences using algorithms, such as BESTFIT, FASTA, and TFASTA in the Wisconsin Genetics Software Package Release 7.0 (Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occur in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated, the window of comparison between two sequences is defined by the entire length of the shorter of the two sequences. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal, or internal extensions, deletions, or insertions into the peptide sequence shall not be construed as affecting homology.

A “target nucleic acid” is a nucleic acid into which a transposon is to be inserted. Such a target can be part of a chromosome, episome, or vector.

An “integration target sequence” or “target sequence” or “target site” for a transposase is a site or sequence in a target DNA molecule into which a transposon can be inserted by a transposase. The piggyBac transposase from Trichoplusia ni inserts its transposon predominantly into the target sequence 5′-TTAA-3′. PiggyBac-like transposases transpose their transposons using a cut-and-paste mechanism, which results in duplication of their 4 base pair target sequence on insertion into a DNA molecule. The target sequence is thus found on each side of an integrated piggyBac-like transposon.

The term “translation” refers to the process by which a polypeptide is synthesized by a ribosome “reading” the sequence of a polynucleotide.

A “transposase” is a polypeptide that catalyzes the excision of a corresponding transposon from a donor polynucleotide, for example a vector, and (providing the transposase is not integration-deficient) the subsequent integration of the transposon into a target nucleic acid. A transposase may be a piggyBac-like transposase. Other non-limiting, suitable transposases are disclosed in U.S. Pat. No. 10,041,077B2, which is incorporated herein by reference in its entirety.

The term “transposition” refers to the action of a transposase in excising a transposon from one polynucleotide and then integrating it, either into a different site in the same polynucleotide, or into a second polynucleotide.

The term “transposon” means a polynucleotide that can be excised from a first polynucleotide, for instance, a vector, and be integrated into a second position in the same polynucleotide, or into a second polynucleotide, for instance, the genomic or extrachromosomal DNA of a cell, by the action of a corresponding trans-acting transposase. A transposon comprises a first transposon end and a second transposon end, which are polynucleotide sequences recognized by and transposed by a transposase. A transposon usually further comprises a first polynucleotide sequence between the two transposon ends, such that the first polynucleotide sequence is transposed along with the two transposon ends by the action of the transposase. Natural transposons frequently comprise DNA encoding a transposase that acts on the transposon. Transposons as claimed herein are “synthetic transposons,” comprising a heterologous polynucleotide sequence that is transposable by virtue of its juxtaposition between two transposon ends. A suitable transposon is a piggyBac-like transposon. Other non-limiting, suitable transposons are disclosed in U.S. Pat. No. 10,041,077B2.

The term “transposon end” means the cis-acting nucleotide sequences that are sufficient for recognition by and transposition by a corresponding transposase. Transposon ends of piggyBac-like transposons comprise perfect or imperfect repeats such that the respective repeats in the two transposon ends are reverse complements of each other. These are referred to as ITRs or terminal inverted repeats (“TIR”s). A transposon end may or may not include an additional sequence proximal to the ITR that promotes or augments transposition.

The term “vector,” “DNA vector,” or “gene transfer vector” refers to a polynucleotide that is used to perform a “carrying” function for another polynucleotide. For example, vectors are often used to allow a polynucleotide to be propagated within a living cell, to allow a polynucleotide to be packaged for delivery into a cell, or to allow a polynucleotide to be integrated into the genomic DNA of a cell. A vector may further comprise additional functional elements, such as, for example, a transposon.

The disclosure refers to several genes and proteins for which it provides an example “SEQ ID NO:” representing the wildtype sequence or a variant of the gene or protein. Unless otherwise apparent from the context, reference to a gene or protein should be understood as including the specific SEQ ID NO:, as well as allelic, species, and induced variants thereof having at least 90, 95, or 99% identity thereto. Examples of allelic and species variants can be found in the SwissProt and other databases.

Mutations are sometimes referred to in the form XnY, wherein X is a wildtype amino acid, n is an amino acid position of X in a wildtype sequence, and Y is a replacement amino acid. If the mutation occurs in a sequence having a different number of amino acids than the wildtype sequence, it is present at the position in the sequence aligned with position n in the wildtype sequence when the respective sequences are maximally aligned.

### II. Transposon Elements

Heterologous polynucleotides may be more efficiently integrated into a target genome if they are part of a transposon, so that they may be integrated by a transposase. A particular benefit of a transposon is that the entire polynucleotide between the transposon ITRs is integrated. This is in contrast with random integration, where a polynucleotide introduced into a eukaryotic cell is often fragmented at random in the cell, and only parts of the polynucleotide become incorporated into the target genome, usually at a low frequency. There are several different classes of transposon. piggyBac-like transposons include the piggyBac transposon from the looper moth Trichoplusia ni, Xenopus piggyBac-like transposons, Bombyx piggyBac-like transposons, Heliothis piggyBac-like transposons, Helicoverpa piggyBac-like transposons, Agrotis piggyBac-Ikike transposons, Amyelois piggyBac-like transposons, piggyBat piggyBac-like transposons, and Oryzias piggyBac-like transposons. hAT transposons include TcBuster. Mariner transposons include Sleeping Beauty. Each of these transposons can be integrated into the genome of a mammalian cell by a corresponding transposase. Heterologous polynucleotides incorporated into transposons may be integrated into mammalian cells, as well as hepatocytes, neural cells, muscle cells, blood cells, embryonic stem cells, somatic stem cells, hematopoietic cells, embryos, zygotes, and sperm cells (some of which are open to being manipulated in an in vitro setting). Cells can also be pluripotent cells (cells whose descendants can differentiate into several restricted cell types, such as hematopoietic stem cells or other stem cells) or totipotent cells (i.e., a cell whose descendants can become any cell type in an organism, e.g., embryonic stem cells).

Gene transfer systems may comprise a transposon in combination with a corresponding transposase protein that transposases the transposon, or a nucleic acid that encodes the corresponding transposase protein and is expressible in the target cell. The nucleic acid encoding the transposase protein may be a DNA molecule or an mRNA molecule.

When there are multiple components of a gene transfer system, for example one or more polynucleotides comprising transposon ends flanking genes for expression in the target cell, and a transposase (which may be provided either as a protein or encoded by a nucleic acid), these components can be transfected into a cell at the same time, or sequentially. For example, a transposase protein or its encoding nucleic acid may be transfected into a cell prior to, at the same time, or after transfection of a corresponding transposon. Additionally, administration of either component of the gene transfer system may occur repeatedly, for example, by administering at least two doses of this component.

Transposase proteins may be encoded by polynucleotides including RNA or DNA. RNA molecules may include those with appropriate substitutions to reduce toxicity effects on the cell, such as, for example, substitution of uridine with pseudouridine and substitution of cytosine with 5-methyl cytosine. mRNA encoding the transposase may be prepared such that it has a 5′-cap structure to improve expression in a target cell. Example cap structures include a cap analog (G(5′)ppp(5′)G), an anti-reverse cap analog (3′-O-Me-m⁷G(5′)ppp(5′)G, a clean cap (m7G(5′)ppp(5′)(2′OmeA)pG), and an mCap (m7G(5′)ppp(5′)G). mRNA encoding the transposase may be prepared such that some bases are partially or fully substituted, for example, uridine may be substituted with pseudo-uridine, and cytosine may be substituted with 5-methyl-cytosine. Any combinations of these caps and substitutions may be made. Similarly, the nucleic acid encoding the transposase protein or the corresponding transposon can be transfected into the cell as a linear fragment or as a circularized fragment, either as a plasmid or as recombinant viral DNA. If the transposase is introduced as a DNA sequence encoding the transposase, then the ORF encoding the transposase may be operably linked to a promoter that is active in the target mammalian cell.

A suitable piggyBac-like transposon for modifying the genome of a mammalian cell is a Xenopus transposon, which comprises, from 5′ to 3′, a first ITR with the nucleotide sequence SEQ ID NO: 1, a heterologous polynucleotide to be transposed, and a second ITR with the nucleotide sequence SEQ ID NO: 2. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 5 or 6. The transposon may further comprise a second additional polynucleotide immediately adjacent to one ITR, e.g., the second ITR, and proximal to the heterologous polynucleotide, whose nucleotide sequence is at least 95% identical to SEQ ID NO: 7 or 8. This transposon may be transposed by a corresponding Xenopus transposase comprising a polypeptide sequence at least 90% identical to the polypeptide sequence of SEQ ID NO: 9 or 10, for example any of SEQ ID NOs: 9-41. The Xenopus transposase may optionally be fused to a heterologous nuclear localization signal. The transposase may be a hyperactive variant of a naturally occurring transposase. The hyperactive variant transposase may comprise one or more of the following amino acid changes, relative to the polypeptide sequence of SEQ ID NO: 9: Y6L, Y6H, Y6V, Y6I, Y6C, Y6G, Y6A, Y6S, Y6F, Y6R, Y6P, Y6D, Y6N, S7G, S7V, S7D, E9W, E9D, E9E, M16E, M16N, M16D, M16S, M16Q, M16T, M16A, M16L, M16H, M16F, M16I, S18C, S18Y, S18M, S18L, S18Q, S18G, S18P, S18A, S18W, S18H, S18K, S18I, S18V, S19C, S19V, S19L, S19F, S19K, S19E, S19D, S19G, S19N, S19A, S19M, S19P, S19Y, S19R, S19T, S19Q, S20G, S20M, S20L, S20V, S20H, S20W, S20A, S20C, S20Q, S20D, S20F, S20N, S20R, E21N, E21W, E21G, E21Q, E21L, E21D, E21A, E21P, E21T, E21S, E21Y, E21V, E21F, E21M, E22C, E22H, E22R, E22L, E22K, E22S, E22G, E22M, E22V, E22Q, E22A, E22Y, E22W, E22D, E22T, F23Q, F23A, F23D, F23W, F23K, F23T, F23V, F23M, F23N, F23P, F23H, F23E, F23C, F23R, F23Y, S24L, S24W, S24H, S24V, S24P, S24I, S24F, S24K, S24Y, S24D, S24C, S24N, S24G, S24A, S26F, S26H, S26V, S26Q, S26Y, S26W, S28K, S28Y, S28C, S28M, S28L, S28H, S28T, S28Q, V31L, V31T, V31I, V31Q, V31K, A34L, A34E, L67A, L67T, L67M, L67V, L67C, L67H, L67E, L67Y, G73H, G73N, G73K, G73F, G73V, G73D, G73S, G73W, G73L, A76L, A76R, A76E, A76I, A76V, D77N, D77Q, D77Y, D77L, D77T, P88A, P88E, P88N, P88H, P88D, P88L, N91D, N91R, N91A, N91L, N91H, N91V, Y1411, Y141M, Y141Q, Y141S, Y141E, Y141W, Y141V, Y141F, Y141A, Y141C, Y141K, Y141L, Y141H, Y141R, N145C, N145M, N145A, N145Q, N145I, N145F, N145G, N145D, N145E, N145V, N145H, N145W, N145Y, N145L, N145R, N145S, P146V, P146T, P146W, P146C, P146Q, P146L, P146Y, P146K, P146N, P146F, P146E, P148M, P148R, P148V, P148F, P148T, P148C, P148Q, P148H, Y150W, Y150A, Y150F, Y150H, Y150S, Y150V, Y150C, Y150M, Y150N, Y150D, Y150E, Y150Q, Y150K, H157Y, H157F, H157T, H157S, H157W, A162L, A162V, A162C, A162K, A162T, A162G, A162M, A162S, A162I, A162Y, A162Q, A179T, A179K, A179S, A179V, A179R, L182V, L182I, L182Q, L182T, L182W, L182R, L182S, T189C, T189N, T189L, T189K, T189Q, T189V, T189A, T189W, T189Y, T189G, T189F, T189S, T189H, L192V, L192C, L192H, L192M, L1921, S193P, S193T, S193R, S193K, S193G, S193D, S193N, S193F, S193H, S193Q, S193Y, V196L, V196S, V196W, V196A, V196F, V196M, V196I, S198G, S198R, S198A, S198K, T200C, T2001, T200M, T200L, T200N, T200W, T200V, T200Q, T200Y, T200H, T200R, S202A, S202P, L210H, L210A, F212Y, F212N, F212M, F212C, F212A, N218V, N218R, N218T, N218C, N218G, N218I, N218P, N218D, N218E, A248S, A248L, A248H, A248C, A248N, A248I, A248Q, A248Y, A248M, A248D, L263V, L263A, L263M, L263R, L263D, Q270V, Q270K, Q270A, Q270C, Q270P, Q270L, Q270I, Q270E, Q270G, Q270Y, Q270N, Q270T, Q270W, Q270H, S294R, S294N, S294G, S294T, S294C, T297C, T297P, T297V, T297M, T297L, T297D, E304D, E304H, E304S, E304Q, E304C, S308R, S308G, L310R, L310I, L310V, L333M, L333W, L333F, Q336Y, Q336N, Q336M, Q336A, Q336T, Q336L, Q336I, Q336G, Q336F, Q336E, Q336V, Q336C, Q336H, A354V, A354W, A354D, A354C, A354R, A354E, A354K, A354H, A354G, C357Q, C357H, C357W, C357N, C357I, C357V, C357M, C357R, C357F, C357D, L358A, L358F, L358E, L358R, L358Q, L358V, L358H, L358C, L358M, L358Y, L358K, L358N, L358I, D359N, D359A, D359L, D359H, D359R, D359S, D359Q, D359E, D359M, L377V, L377I, V423N, V423P, V423T, V423F, V423H, V423C, V423S, V423G, V423A, V423R, V423L, P426L, P426K, P426Y, P426F, P426T, P426W, P426V, P426C, P426S, P426Q, P426H, P426N, K428R, K428Q, K428N, K428T, K428F, S434A, S434T, S438Q, S438A, S438M, T447S, T447A, T447C, T447Q, T447N, T447G, L450M, L450V, L450A, L450I, L450E, A462M, A462T, A462Y, A462F, A462K, A462R, A462Q, A462H, A462E, A462N, A462C, V467T, V467C, V467A, V467K, 1469V, 1469N, 1472V, 1472L, 1472W, 1472M, 1472F, L476I, L476V, L476N, L476F, L476M, L476C, L476Q, P488E, P488H, P488K, P488Q, P488F, P488M, P488L, P488N, P488D, Q498V, Q498L, Q498G, Q498H, Q498T, Q498C, Q498E, Q498M, L502I, L502M, L502V, L502G, L502F, E517M, E517V, E517A, E517K, E517L, E517G, E517S, E517I, P520W, P520R, P520M, P520F, P520Q, P520V, P520G, P520D, P520K, P520Y, P520E, P520L, P520T, S521A, S521H, S521C, S521V, S521W, S521T, S521K, S521F, S521G, N523W, N523A, N523G, N523S, N523P, N523M, N523Q, N523L, N523K, N523D, N523H, N523F, N523C, 1533M, 1533V, 1533T, I533S, 1533F, 1533G, I533E, D534E, D534Q, D534L, D534R, D534V, D534C, D534M, D534N, D534A, D534G, D534F, D534T, D534H, D534K, D534S, F576L, F576K, F576V, F576D, F576W, F576M, F576C, F576R, F576Q, F576A, F576Y, F576N, F576G, F576I, F576E, K577L, K577G, K577D, K577R, K577H, K577Y, K577I, K577E, K577V, K577N, 1582V, 1582K, 1582R, I582M, 1582G, 1582N, 1582E, 1582A, 1582Q, Y583L, Y583C, Y583F, Y583D, Y583Q, L587F, L587D, L587R, L5871, L587P, L587N, L587E, L587S, L587Y, L587M, L587Q, L587G, L587W, L587K or L587T.

A suitable piggyBac-like transposon for modifying the genome of a mammalian cell is a Bombyx transposon, which comprises, from 5′ to 3′, a first ITR with the nucleotide sequence SEQ ID NO: 42, a heterologous polynucleotide to be transposed, and a second ITR with the nucleotide sequence SEQ ID NO: 43. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 44. The transposon may further comprise a second additional polynucleotide immediately adjacent to one ITR, e.g., the second ITR, and proximal to the heterologous polynucleotide, whose nucleotide sequence is at least 95% identical to SEQ ID NO: 45. This transposon may be transposed by a corresponding Bombyx transposase comprising a polypeptide sequence at least 90% identical to the polypeptide sequence of SEQ ID NO: 46 or 47, for example any of SEQ ID NOs: 46-69. The Bombyx transposase may optionally be fused to a heterologous nuclear localization signal. The transposase may be a hyperactive variant of a naturally occurring transposase. The hyperactive variant transposase may comprise one or more of the following amino acid changes, relative to the polypeptide sequence of SEQ ID NO: 46: Q85E, Q85M, Q85K, Q85H, Q85N, Q85T, Q85F, Q85L, Q92E, Q92A, Q92P, Q92N, Q92I, Q92Y, Q92H, Q92F, Q92R, Q92D, Q92M, Q92W, Q92C, Q92G, Q92L, Q92V, Q92T, V93P, V93K, V93M, V93F, V93W, V93L, V93A, V93I, V93Q, P96A, P96T, P96M, P96R, P96G, P96V, P96E, P96Q, P96C, F97Q, F97K, F97H, F97T, F97C, F97W, F97V, F97E, F97P, F97D, F97A, F97R, F97G, F97N, F97Y, H165E, H165G, H165Q, H165T, H165M, H165V, H165L, H165C, H165N, H165D, H165K, H165W, H165A, E178S, E178H, E178Y, E178F, E178C, E178A, E178Q, E178G, E178V, E178D, E178L, E178P, E178W, C189D, C189Y, C189I, C189W, C189T, C189K, C189M, C189F, C189P, C189Q, C189V, A196G, L2001, L200F, L200C, L200M, L200Y, A201Q, A201L, A201M, L203V, L203D, L203G, L203E, L203C, L203T, L203M, L203A, L203Y, N207G, N207A, L211G, L211M, L211C, L211T, L211V, L211A, W215Y, T217V, T217A, T217I, T217P, T217C, T217Q, T217M, T217F, T217D, T217K, G219S, G219A, G219C, G219H, G219Q, Q235C, Q235N, Q235H, Q235G, Q235W, Q235Y, Q235A, Q235T, Q235E, Q235M, Q235F, Q238C, Q238M, Q238H, Q238V, Q238L, Q238T, Q238I, R242Q, K246I, K253V, M258V, F261L, S263K, C271S, N303C, N303R, N303G, N303A, N303D, N303S, N303H, N303E, N303R, N303K, N303L, N303Q, 1312F, 1312C, 1312A, 1312L, 1312T, 1312V, 1312G, 1312M, F321H, F321R, F321N, F321Y, F321W, F321D, F321G, F321E, F321M, F321K, F321A, F321Q, V323I, V323L, V323T, V323M, V323A, V324N, V324A, V324C, V324I, V324L, V324T, V324K, V324Y, V324H, V324F, V324S, V324Q, V324M, V324G, A330K, A330V, A330P, A330S, A330C, A330T, A330L, Q333P, Q333T, Q333M, Q333H, Q333S, P337W, P337E, P337H, P337I, P337A, P337M, P337N, P337D, P337K, P337Q, P337G, P337S, P337C, P337L, P337V, F368Y, L373C, L373V, L373I, L373S, L373T, V389I, V389M, V389T, V389L, V389A, R394H, R394K, R394T, R394P, R394M, R394A, Q395P, Q395F, Q395E, Q395C, Q395V, Q395A, Q395H, Q395S, Q395Y, S399N, S399E, S399K, S399H, S399D, S399Y, S399G, S399Q, S399R, S399T, S399A, S399V, S399M, R402Y, R402K, R402D, R402F, R402G, R402N, R402E, R402M, R402S, R402Q, R402T, R402C, R402L, R402V, T403W, T403A, T403V, T403F, T403L, T403Y, T403N, T403G, T403C, T4031, T403S, T403M, T403Q, T403K, T403E, D404I, D404S, D404E, D404N, D404H, D404C, D404M, D404G, D404A, D404Q, D404L, D404P, D404V, D404W, D404F, N408F, N4081, N408A, N408E, N408M, N408S, N408D, N408Y, N408H, N408C, N408Q, N408V, N408W, N408L, N408P, N408K, S409H, S409Y, S409N, S409I, S409D, S409F, S409T, S409C, S409Q, N441F, N441R, N441M, N441G, N441C, N441D, N441L, N441A, N441V, N441W, G448W, G448Y, G448H, G448C, G448T, G448V, G448N, G448Q, E449A, E449P, E449T, E449L, E449H, E449G, E449C, E449I, V469T, V469A, V469H, V469C, V469L, L472K, L472Q, L472M, C473G, C473Q, C473T, C473I, C473M, R484H, R484K, T507R, T507D, T507S, T507G, T507K, T507I, T507M, T507E, T507C, T507L, T507V, G523Q, G523T, G523A, G523M, G523S, G523C, G523I, G523L, 1527M, 1527V, Y528N, Y528W, Y528M, Y528Q, Y528K, Y528V, Y5281, Y528G, Y528D, Y528A, Y528E, Y528R, Y543C, Y543W, Y5431, Y543M, Y543Q, Y543A, Y543R, Y543H, E549K, E549C, E549I, E549Q, E549A, E549H, E549C, E549M, E549S, E549F, E549L, K550R, K550M, K550Q, S556G, S556V, S556I, P557W, P557T, P557S, P557A, P557Q, P557K, P557D, P557G, P557N, P557L, P557V, H559K, H559S, H559C, H559I, H559W, V560F, V560P, V560I, V560H, V560Y, V560K, N561P, N561Q, N561G, N561A, V562Y, V562I, V562S, V562M, V567I, V567H, V567N, S583M, E601V, E601F, E601Q, E601W, E605R, E605W, E605K, E605M, E605P, E605Y, E605C, E605H, E605A, E605Q, E605S, E605V, E605I, E605G, D607V, D607Y, D607C, D607N, D607W, D607T, D607A, D607H, D607Q, D607E, D607L, D607K, D607G, S609R, S609W, S609H, S609V, S609Q, S609G, S609T, S609K, S609N, S609Y, L610T, L610I, L610K, L610G, L610A, L610W, L610D, L610Q, L610S, L610F or L610N.

A suitable piggyBac-like transposon for modifying the genome of a mammalian cell is a Myotis transposon, which comprises, from 5′ to 3′, a first ITR with the nucleotide sequence SEQ ID NO: 70, a heterologous polynucleotide to be transposed, and a second ITR with the nucleotide sequence SEQ ID NO: 71. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 72. The transposon may further comprise a second additional polynucleotide immediately adjacent to one ITR, e.g., the second ITR, and proximal to the heterologous polynucleotide, whose nucleotide sequence is at least 95% identical to SEQ ID NO: 73. This transposon may be transposed by a corresponding Myotis transposase comprising a polypeptide sequence at least 90% identical to the polypeptide sequence of SEQ ID NO: 74. The Myotis transposase may optionally be fused to a heterologous nuclear localization signal. The transposase may be a hyperactive variant of a naturally occurring transposase. The hyperactive variant transposase may comprise one or more of the following amino acid changes, relative to the sequence of SEQ ID NO: 74: A14V, D475G, P491Q. A56IT. T546T, T300A, T294A, A520T, G239S, S5P, S8F. S54N. D9N, D9G. 1345 V, M481V, EI1G, K130T. G9G, R427H, S8P, S36G, DIOG, S36G.

A suitable piggyBac-like transposon for modifying the genome of a mammalian cell is a Trichoplusia transposon, which comprises, from 5′ to 3′, a first ITR with the nucleotide sequence SEQ ID NO: 75, a heterologous polynucleotide to be transposed, and a second ITR with the nucleotide sequence SEQ ID NO: 76. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 77. The transposon may further comprise a second additional polynucleotide immediately adjacent to one ITR, e.g., the second ITR, and proximal to the heterologous polynucleotide, whose nucleotide sequence is at least 95% identical to SEQ ID NO: 78. This transposon may be transposed by a corresponding Trichoplusia transposase comprising a polypeptide sequence at least 90% identical to the polypeptide sequence of SEQ ID NO: 79. The Trichoplusia transposase may optionally be fused to a heterologous nuclear localization signal. The transposase may be a hyperactive variant of a naturally occurring transposase. The hyperactive variant transposase may comprise one or more of the following amino acid changes, relative to the sequence of SEQ ID NO: 79: G2C, Q40R, 130V, G165S, T43A, S61R, S103P, S103T, M194V, R281G, M282V, G316E, 1426V, Q497L, N505D, Q573L, S509G, N570S, N538K, Q591P, Q591R, F594L, M194V, 130V, S103P, G165S, M282V, S509G, N538K, N571S, C4IT, A1424G, C1472A, G1681A, T150C, A351G, A279G, T1638C, A898G, A880G, G1558A, A687G, G715A, T13C, C23T, G161A, G25A, T1050C, A1356G, A26G, A1033G, A1441G, A32G, A389C, A32G, A389C, A32G, T1572A, G456A, T1641C, TI 155C, G1280A, T22C, A106G, A29G, C137T, A14V, D475G, P491Q, A561T, T546T, T300A, T294A, A520T, G239S, S5P, S8F, S54N, DON, D9G, 1345 V, M481V, E11G, K130T, G9G, R427H, S8P, S36G, DIOG, S36G, A51T, C153A, C277T, G201A, G202A, T236A, A103T, A104C, T140C, G138T, T118A, C74T, A179C, S3N, I30V, A46S, A46T, 182W, S103P, R119P, C125A, C125L, G165S, Y177K, Y177H, F180L, F180I, F180V, M185L, A187G, F200W, V207P, V209F, M226F, L235R, V240K, F241L, P243K, N258S, M282Q, L296W, L296Y, L296F, M298V, M298A, M298L, P311V, P311I, R315K, T319G, Y327R, Y328V, C340G, C340L, D421H, V436I, M456Y, L470F, S486K, M503I, M503L, V552K, A570T, Q591P, Q591R, R65A, R65E, R95A, R95E, R97A, R97E, R135A, R135E, R161A, R161E, R192A, R192E, R208A, R208E, K176A, K176E, K195A, K195E, S171E, M14V, D270N, 130V, G165S, M282L, M282I, M282V or M282A.

A suitable piggyBac-like transposon for modifying the genome of a mammalian cell is an Amyelois transposon, which comprises, from 5′ to 3′, a first ITR with the nucleotide sequence SEQ ID NO: 80, a heterologous polynucleotide to be transposed, and a second ITR with the nucleotide sequence SEQ ID NO: 81. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 82. The transposon may further comprise a second additional polynucleotide immediately adjacent to one ITR, e.g., the second ITR, and proximal to the heterologous polynucleotide, whose nucleotide sequence is at least 95% identical to SEQ ID NO: 83. This transposon may be transposed by a corresponding Amyelois transposase comprising a polypeptide sequence at least 90% identical to the polypeptide sequence of SEQ ID NO: 84. The Amyelois transposase may optionally be fused to a heterologous nuclear localization signal. The transposase may be a hyperactive variant of a naturally occurring transposase. The hyperactive variant transposase may comprise one or more of the following amino acid changes, relative to the sequence of SEQ ID NO: 84: P65E, P65D, R95S, R95T, V100I, V100L, V100M, L115D, L115E, E116P, H121Q, H121N, K139E, K139D, T159N, T159Q, V166F, V166Y, V166W, G179N, G179Q, W187F, W187Y, P198R, P198K, L203R, L203K, I209L, I209V, I209M, N211R, N211K, E238D, L273I, L273V, L273M, D304K, D304R, 1323L, 1323M, 1323V, Q329G, Q329R, Q329K, T345L, T345I, T345V, T345M, K362R, T366R, T366K, T380S, L408M, L4081, L408V, E413S, E413T, S416E, S416D, 1426M, I426L, I426V, S435G, L458M, L458I, L458V, A472S, A472T, V475I, V475L, V475M, N483K, N483R, 1491M, 1491V, 1491L, A529P, K540R, S560K, S560R, T562K, T562R, S563K, S563R.

A suitable piggyBac-like transposon for modifying the genome of a mammalian cell is a Heliothis transposon, which comprises, from 5′ to 3′, a first ITR with the nucleotide sequence SEQ ID NO: 85, a heterologous polynucleotide to be transposed, and a second ITR with the nucleotide sequence SEQ ID NO: 86. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 87. The transposon may further comprise a second additional polynucleotide immediately adjacent to one ITR, e.g., the second ITR, and proximal to the heterologous polynucleotide, whose nucleotide sequence is at least 95% identical to SEQ ID NO: 88. This transposon may be transposed by a corresponding Heliothis transposase comprising a polypeptide sequence at least 90% identical to the polypeptide sequence of SEQ ID NO: 89. The Heliothis transposase may optionally be fused to a heterologous nuclear localization signal. The transposase may be a hyperactive variant of a naturally occurring transposase. The hyperactive variant transposase may comprise one or more of the following amino acid changes, relative to the sequence of SEQ ID NO: 89: S41V, S41I, S41L, L43S, L43T, V81E, V81D, D83S, D83T, V85L, V85I, V85M, P125S, P125T, Q126S, Q126T, Q131R, Q131K, Q131T, Q131S, S136V, S136I, S136L, S136M, E140C, E140A, N151Q, K169E, K169D, N212S, 1239L, 1239V, 1239M, H241N, H241Q, T268D, T268E, T297C, M300R, M300K, M305N, M305Q, L312I, C316A, C316M, L321V, L321M, N322T, N322S, P351G, H357R, H357K, H357D, H357E, K360Q, K360N, E379P, K397S, K397T, Y421F, Y421W, V450I, V450L, V450M, Y495F, Y495W, A447N, A447D, A449S, A449V, K476L, V492A, 1500M, L585K and T595K.

A suitable piggyBac-like transposon for modifying the genome of a mammalian cell is an Oryzias transposon which comprises, from 5′ to 3′, a first ITR with the nucleotide sequence SEQ ID NO: 90 or SEQ ID NO: 92, a heterologous polynucleotide to be transposed, and a second ITR with the nucleotide sequence SEQ ID NO: 91 or SEQ ID NO: 93. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 94. The transposon may further comprise a second additional polynucleotide immediately adjacent to one ITR, e.g., the second ITR, and proximal to the heterologous polynucleotide, whose nucleotide sequence is at least 95% identical to SEQ ID NO: 95. This transposon may be transposed by a corresponding Oryzias transposase comprising a polypeptide sequence at least 90% identical to the polypeptide sequence of SEQ ID NO: 96. The Oryzias transposase may optionally be fused to a heterologous nuclear localization signal. The transposase may be a hyperactive variant of a naturally occurring transposase. The hyperactive variant transposase may comprise one or more of the following amino acid changes, relative to the sequence of SEQ ID NO: 96: E22D, A124C, Q131D, Q131E, L138V, L138I, L138M, D160E, Y164F, Y164W, 1167L, 1167V, 1167M, T202R, T202K, I206L, I206V, I206M, I210L, 1210V, 1210M, N214D, N214E, V253I, V253L, V253M, V258L, V258I, V258M, A284L, A284I, A284M, A284V, V386I, V386M, V386L, M400L, M400I, M400V, S408E, S408D, L409I, L409V, L409M, V458L, V458M, V458I, V467I, V467M, V467L, L468I, L468V, L468M, A514R, A514K, V515I, V515M, V515L, R548K, D549K, D549R, D550R, D550K, S551K and S551R

A suitable piggyBac-like transposon for modifying the genome of a mammalian cell is an Agrotis transposon, which comprises, from 5′ to 3′, a first ITR with the nucleotide sequence SEQ ID NO: 97, a heterologous polynucleotide to be transposed, and a second ITR with the nucleotide sequence SEQ ID NO: 98. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 99. The transposon may further comprise a second additional polynucleotide immediately adjacent to one ITR, e.g., the second ITR, and proximal to the heterologous polynucleotide, whose nucleotide sequence is at least 95% identical to SEQ ID NO: 100. This transposon may be transposed by a corresponding Agrotis transposase comprising a polypeptide sequence at least 90% identical to the polypeptide sequence of SEQ ID NO: 101. The Agrotis transposase may optionally be fused to a heterologous nuclear localization signal. The transposase may be a hyperactive variant of a naturally occurring transposase.

A suitable piggyBac-like transposon for modifying the genome of a mammalian cell is a Helicoverpa transposon, which comprises, from 5′ to 3′, a first ITR with the nucleotide sequence SEQ ID NO: 102, a heterologous polynucleotide to be transposed, and a second ITR with the nucleotide sequence SEQ ID NO: 103. The transposon may further be flanked by a copy of the tetranucleotide 5′-TTAA-3′ on each side, immediately adjacent to the ITRs and distal to the heterologous polynucleotide. The transposon may further comprise a first additional polynucleotide immediately adjacent to one ITR, e.g., the first ITR, and proximal to the heterologous polynucleotide, whose nucleotide sequence is at least 95% identical to SEQ ID NO: 104. The transposon may further comprise a second additional polynucleotide immediately adjacent to one ITR, e.g., the second ITR, and proximal to the heterologous polynucleotide, whose nucleotide sequence is at least 95% identical to SEQ ID NO: 105. This transposon may be transposed by a corresponding Helicoverpa transposase comprising a polypeptide sequence at least 90% identical to the polypeptide sequence of SEQ ID NO: 106. The Helicoverpa transposase may optionally be fused to a heterologous nuclear localization signal. The transposase may be a hyperactive variant of a naturally occurring transposase.

A suitable Mariner transposon for modifying the genome of a mammalian cell is a Sleeping Beauty transposon, which comprises, from 5′ to 3′, a first ITR with the with nucleotide sequence of SEQ ID NO: 107, a heterologous polynucleotide to be transposed, and a second ITR with nucleotide sequence of SEQ ID NO: 108. The transposon may further comprise a first additional polynucleotide immediately adjacent to one ITR, e.g., the first ITR, and proximal to the heterologous polynucleotide, whose nucleotide sequence is at least 95% identical to SEQ ID NO: 109. The transposon may further comprise a second additional polynucleotide immediately adjacent to one ITR, e.g., the second ITR, and proximal to the heterologous polynucleotide, whose nucleotide sequence is at least 95% identical to SEQ ID NO: 110. This transposon may be transposed by a corresponding Sleeping Beauty transposase comprising a polypeptide sequence at least 90% identical to the polypeptide sequence of SEQ ID NO: 111, including hyperactive variants thereof.

A suitable hAT transposon for modifying the genome of a mammalian cell is a TcBuster transposon, which comprises, from 5′ to 3′, a first ITR with the nucleotide sequence SEQ ID NO: 112, a heterologous polynucleotide to be transposed, and a second ITR with the nucleotide sequence SEQ ID NO: 113. The transposon may further comprise a first additional polynucleotide immediately adjacent to one ITR, e.g., the first ITR, and proximal to the heterologous polynucleotide, whose nucleotide sequence is at least 95% identical to SEQ ID NO: 114. The transposon may further comprise a second additional polynucleotide immediately adjacent to one ITR, e.g., the second ITR, and proximal to the heterologous polynucleotide, whose nucleotide sequence is at least 95% identical to SEQ ID NO: 115. This transposon may be transposed by a corresponding Sleeping Beauty transposase comprising a polypeptide sequence at least 90% identical to the polypeptide sequence of SEQ ID NO: 116, including hyperactive variants thereof.

A transposase protein can be introduced into a cell as a protein or as a nucleic acid encoding the transposase, for example as a ribonucleic acid, including mRNA or any polynucleotide recognized by the translational machinery of a cell; as DNA, e.g., as extrachromosomal DNA including episomal DNA; as plasmid DNA, or as viral nucleic acid. Furthermore, the nucleic acid encoding the transposase protein can be transfected into a cell as a nucleic acid vector such as a plasmid, or as a gene expression vector, including a viral vector. The nucleic acid can be circular or linear. DNA encoding the transposase protein can be stably inserted into the genome of the cell or into a vector for constitutive or inducible expression. Where the transposase protein is transfected into the cell or inserted into the vector as DNA, the transposase encoding sequence may be operably linked to a heterologous promoter. There are a variety of promoters that could be used, including constitutive promoters, tissue-specific promoters, inducible promoters, species-specific promoters, cell-type specific promoters, and the like. All DNA or RNA sequences encoding transposase proteins are expressly contemplated. Alternatively, the transposase may be introduced into the cell directly as protein, for example using cell-penetrating peptides (e.g., as described in Ramsey and Flynn, 2015. Pharmacol. Ther. 154: 78-86 “Cell-penetrating peptides transport therapeutics into cells”); using small molecules including salt plus propanebetaine (e.g., as described in Astolfo et. Al., 2015. Cell 161: 674-690); or electroporation (e.g., as described in Morgan and Day, 1995. Methods in Molecular Biology 48: 63-71 “The introduction of proteins into mammalian cells by electroporation”).

### III. Split DHFR Selection Systems

In one aspect, systems are provided for selecting cells that have undergone gene transfer successfully with two different DNA molecules. In brief, expression units for the two DHFR^{FS} fragments are placed on two separate DNA molecules, the two DNA molecules are introduced into cells by any gene transfer procedure, and methotrexate is used to kill cells that have not become stably modified by both DNA molecules. The method includes the use of fusions of the DHFR^{FS} fragments that dimerize efficiently and stably inside cells.

In one aspect, a single drug (i.e., methotrexate) may be used to select for two exogenously provided DNA molecules. Since many gene transfer methods become inefficient as the size of the transferred DNA molecule increases, the option to distribute DNA cargo over two DNA molecules offers a practical way to overcome size limitations. Splitting the DNA cargo over two DNA molecules also reduces difficulties associated with assembling and propagating large DNA plasmids while simplifying the creation of combinatorial genetic effects. Methotrexate is a drug that has been used to treat humans for many years, and, therefore, considerable experience exists to draw on concerning its safety, especially when its use is primarily, if not exclusively, in the ex vivo setting.

In one aspect, methotrexate may select for cells that carry two distinct DNA transposons. Transposons are of interest because: (i) they integrate in their entirety into transcriptionally active regions of the genome; (ii) they have a large cargo capacity that allows them to accommodate multiple transgenes; and (iii) the transposase enzyme necessary for their integration into the genome can be provided to cells in a regulated fashion such that the average number of transposon integrants per cell can be kept low. Of course, the use of retroviral and lentiviral vectors is also contemplated and is enabled to one having ordinary skill in the art in view of this disclosure.

The split DHFR methodology provides a facile means for using one drug to select cells that contain stable genomic integrations of two such transposons. The method affords a doubling of transgene cargo capacity, which significantly extends the possibilities for engineering the genome/exome of therapeutic cells.

### EXAMPLES

### Example 1: Split DHFR Selection Systems

The original description of the split DHFR system included a demonstration that a leucine zipper sequence from yeast (taken from the GCN4 transcription factor) could be used to reconstitute DHFR activity in E. Coli. See Pelletier J N, Campbell-Valois F X, Michnick S W. Oligomerization domain-directed reassembly of active dihydrofolate reductase from rationally designed fragments. Proc Natl Acad Sci USA. 1998; 95 (21): 12141-12146. doi:10.1073/pnas.95.21.12141. This same strategy has been subsequently employed to show efficacy of the split DHFR system in Plasmodium falciparum. See Levray Y S, Berhe A D, Osborne A R. Use of split-dihydrofolate reductase for the detection of protein-protein interactions and simultaneous selection of multiple plasmids in Plasmodium falciparum. Mol Biochem Parasitol 2020; 238:111292.doi: 10.1016/j.molbiopara.2020.111292. Importantly, this latter study included a demonstration that the split DHFR could be used as the basis for simultaneous selection of two plasmids using a single antifolate drug. In both of these reports, the murine form of DHFR was used as the source of the two fragments of the enzyme fused to the GCN4 leucine zipper sequence.

In this example, three forms of split DHFR were generated for testing in human T cells (SEQ ID NOs: 117-122):

| SEQ | |
|---|---|
| ID NO: | Construct Name & Sequence |
| 117 | GCN4 Leucine Zipper-Linker-Mouse DHFR |
| | (1-105; L22F/F31S) |
| | MNTEAARRSRARKLQRMKQLEDKVEELLSKNYHLENEVARLK |
| | KLVGERGGGGSGGGGSVRPLNCIVAVSQNMGIGKNGDFPWPP |
| | LRNESKYFQRMTTTSSVEGKQNLVIMGRKTWFSIPEKNRPLK |
| | DRINIVLSRELKEPPRGAHFLAKSLDDALRLIEQPEL* |
| 118 | GCN4 Leucine Zipper-Linker-Mouse DHFR |
| | (106-186) |
| | MNTEAARRSRARKLQRMKQLEDKVEELLSKNYHLENEVARLK |
| | KLVGERGGGGSGGGGSASKVDMVWIVGGSSVYQEAMNQPGHL |
| | RLFVTRIMQEFESDTFFPEIDLGKYKLLPEYPGVLSEVQEEK |
| | GIKYKFEVYEKKD* |
| 119 | N7 Coiled Coil-Linker-Mouse DHFR |
| | (1-105; L22F/F31S) |
| | MGEIAALEAKNAALKAEIAALEAKIAALKAGYGGGGSGGGGS |
| | VRPLNCIVAVSQNMGIGKNGDFPWPPLRNESKYFQRMTTTSS |
| | VEGKQNLVIMGRKTWFSIPEKNRPLKDRINIVLSRELKEPPR |
| | GAHFLAKSLDDALRLIEQPEL* |
| 120 | N8 Coiled Coil-Linker-Mouse DHFR (106-186) |
| | MYGKIAALKAENAALEAKIAALKAEIAALEAGYGGGGSGGGG |
| | SASKVDMVWIVGGSSVYQEAMNQPGHLRLFVTRIMQEFESDT |
| | FFPEIDLGKYKLLPEYPGVLSEVQEEKGIKYKFEVYEKKD* |
| 121 | P7A Coiled Coil-Linker-Mouse DHFR |
| | (1-105; L22F/F31S) |
| | MGEIAALEAKNAALKAEIAALEAKNAALKAGCGGGGSGGGGS |
| | VRPLNCIVAVSQNMGIGKNGDFPWPPLRNESKYFQRMTTTSS |
| | VEGKQNLVIMGRKTWFSIPEKNRPLKDRINIVLSRELKEPPR |
| | GAHFLAKSLDDALRLIEQPEL* |
| 122 | P8A Coiled Coil-Linker-Mouse DHFR |
| | (106-186) |
| | MKIAALKAENAALEAKIAALKAENAALEAGGCGGGGSGGGGS |
| | ASKVDMVWIVGGSSVYQEAMNQPGHLRLFVTRIMQEFESDTF |
| | FPEIDLGKYKLLPEYPGVLSEVQEEKGIKYKFEVYEKKD* |

Each form involved two DNA constructs, one encoding the amino-terminal fragment of murine DHFR and the other encoding the carboxy-terminal fragment of the same enzyme. Three kinds of dimerizing peptides were used: (i) the GCN4 leucine zipper (SEQ ID NO: 185); (ii) the N7/N8 pair of hetero-dimerizing synthetic coiled coil peptides (SEQ ID NOs: 186 and 187); and (iii) the P7A/P8A pair of hetero-dimerizing synthetic coiled coil peptides (SEQ ID NOs: 188 and 189). See Plaper T, Aupič J, Dekleva P, et al. Coiled-coil heterodimers with increased stability for cellular regulation and sensing SARS-COV-2 spike protein-mediated cell fusion. Sci Rep. 2021; 11 (1): 9136. doi: 10.1038/s41598-021-88315-3.

The fusions to the (homo- or hetero-) dimerizing peptides were made at the amino-termini of the DHFR protein fragments. Glycine-serine linker sequences were placed between the dimerizing peptides and the DHFR pieces in all cases. The amino-terminal fragment of DHFR carried L22F and F31S substitutions, which render the enzyme resistant to methotrexate inhibition (at concentrations that are toxic to cells that only express endogenous human DHFR).

Transgenes encoding the six DHFR fusion proteins regulated by the EF1-alpha promoter were each placed in a Leap-In transposon vector that also included a transgene encoding a fluorescent protein. For the transposons expressing the amino-terminal piece of DHFR, the fluorescent protein was mTagBFP2. For the transposons expressing the carboxy-terminal piece, the fluorescent protein was plobRFP. These fluorescent protein transgenes were included so that cells carrying the two kinds of transposons could be readily identified by flow cytometry or fluorescent microscopy.

Jurkat T lymphoma cells were transfected with the appropriate pairs of transposons (i.e., transposons encoding both an amino- and carboxy-terminal piece of DHFR fused to leucine zipper or coiled coil sequences that should associate stably with one another). The ThermoFisher Neon® electroporator was used with 100 μl tips and three pulses of 1350V, each pulse 10 mS in length. 15 μg of plasmid DNA (comprising two plasmids, each encoding different DHFR fragments; i.e., 7.5 μg of each plasmid) were combined with 3 μg of mRNA encoding the Leap-In Transposase® enzyme in each transfection (involving 2 million cells). Electroporations were performed according to the manufacturer's recommendations. The cells were placed into methotrexate-containing medium (RPMI containing 10% (vol/vol) fetal bovine serum and 0.1 μM methotrexate) immediately after transfection and cultured before analysis by flow cytometry.

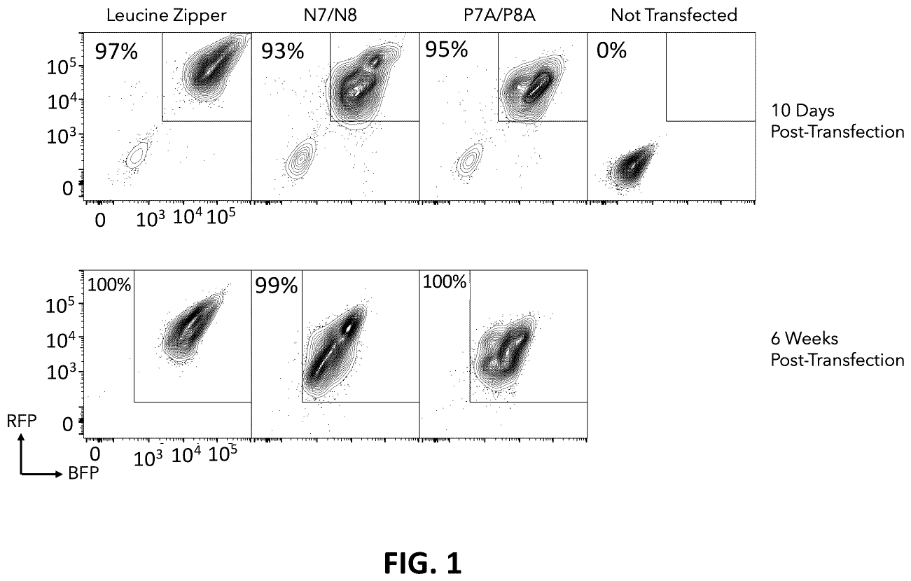

FIG. 1 shows the results of flow cytometric analysis of the cells. Successful methotrexate selection of cells carrying both transfected transposons was evident by the fact that the majority of cells expressed both mTagBFP2 and plobRFP. The cells were analyzed at ten days or six weeks after transfection as indicated.

As shown in FIG. 1, all three pairs of DHFR fragments allowed for the selection of cells that were largely uniform in their co-expression of the two fluorescent proteins (mTagBFP2 and plobRFP). This co-expression indicates that the cells contained both kinds of transposons in each case. Control transfections in which DHFR fragments could not reconstitute a functional enzyme did not support the survival of cells.

The leucine zipper pair of constructs were associated with a higher average level of both red and blue fluorescence than the pairs involving the coiled coil sequences. Since the transgenes controlling expression of the two fluorescent proteins were invariant in the constructs used (and variation in the coding sequences encompassing the DHFR pieces would not be expected to have a direct effect on the transcriptional output of the fluorescent protein transgenes) the increase in mean fluorescence intensity was likely due to more copies of the transposons being present, on average, in the cells that received the leucine zipper pair of constructs. This, in turn, infers that the selection of cells expressing the leucine zipper pairs requires higher levels of the DHFR pieces in cells (as would occur when there are more copies of the transposons) than is true for the coiled coil pairs. This predicts that changing the dimerizing moieties associated with the DHFR pieces can be exploited as a means for achieving different average transposon copy numbers in cells.

The results in FIG. 1 show that the split DHFR system is an effective means for using one drug (methotrexate) to accomplish the selection of cells that carry two distinct DNA molecules (transposons in this case) stably integrated into their genomes.

### Example 2: Generation of a Split DHFR Selection System Based on Human DHFR

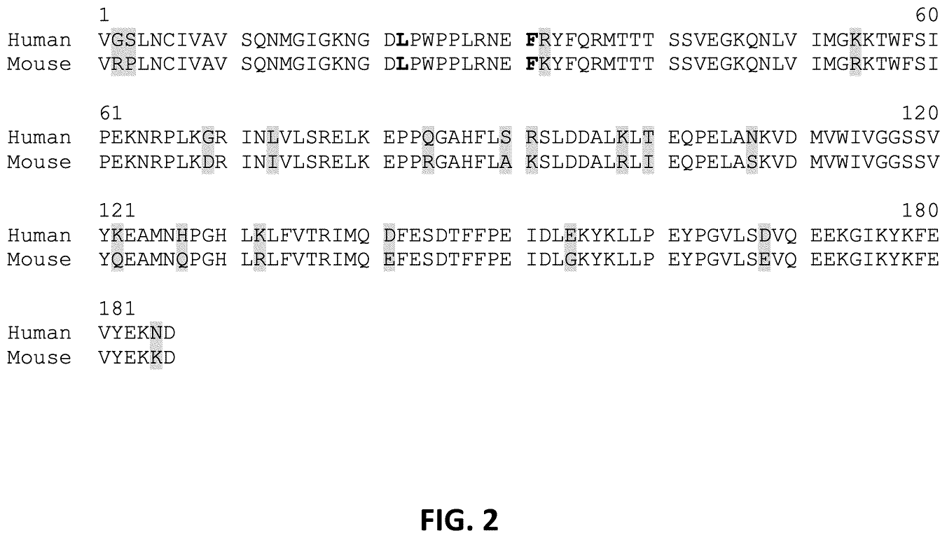

FIG. 2 provides an alignment of the human (SEQ ID NO: 3) and mouse (SEQ ID NO: 4) DHFR protein sequences (involving NCBI reference sequences NP_000782.1 and NP_034179.1 respectively). The protein sequences are shown without the initiator methionine residues, and the numbering convention used throughout this document reflects this elision. Amino acids that differ between the two species are highlighted. Leucine-22 and Phenylalanine-31 are shown in bold, these being the residues that are mutated to Phenylalanine and Serine respectively to create a methotrexate-resistant form of the enzyme (DHFR^{FS}). The mouse and human DHFR proteins are highly similar to one another differing at only 19 of 186 residues (namely, at positions 2, 3, 32, 54, 69, 73, 84, 90, 91, 98, 100, 107, 122, 127, 132, 141, 154, 168 and 185). A split form of human DHFR did not confer resistance to methotrexate in transfected Jurkat cells. This was true even though the human protein was fragmented at the same site as in the functional mouse split enzyme, and despite the use of identical dimerizing peptides. The lack of activity in the human form of split DHFR could not be mitigated by migrating the breakpoint between Proline 103 and Valine 109.

A series of chimeric DHFR fragments was created to determine if a subset of the differences between mouse and human DHFR might be sufficient to render a split form of the human enzyme functional (in terms of conferral of resistance to methotrexate).

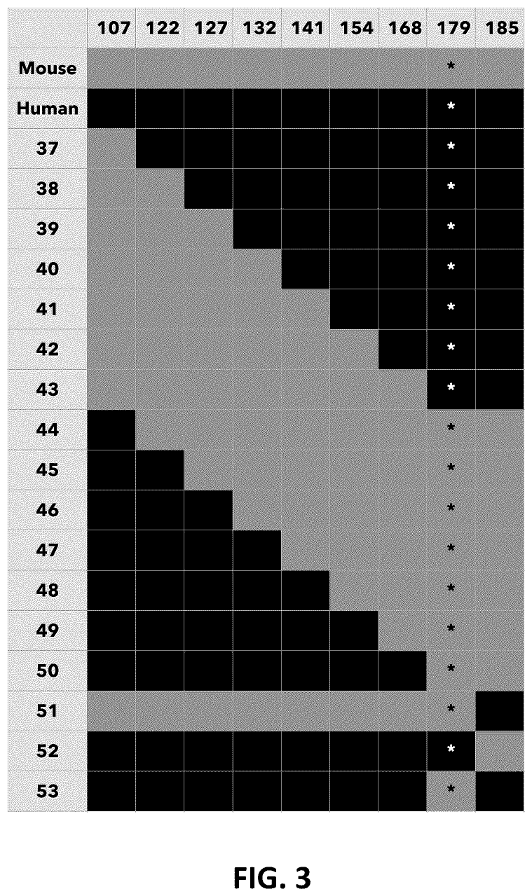

In the first instance, a series of chimeras (variant nos. 37-53, SEQ ID NOs: 123-139) of the carboxy-terminal fragment of DHFR were generated as shown in FIG. 3. Transposons expressing this series of chimeras were transfected into Jurkat cells together with a transposon expressing an amino-terminal fragment that was entirely mouse in protein sequence (but also carried the L22F and F31S substitutions that confer resistance to methotrexate on the enzyme). The transposons also carried transgenes encoding fluorescent proteins: mTagBFP2 was co-expressed from the transposon encoding the amino-terminal DHFR fragment, while plobRFP was co-expressed from the transposon encoding the carboxy-terminal fragment.

Plasmids carrying the transposons were co-transfected with mRNA encoding the Leap-In transposase (the ThermoFisher Neon® electroporator was used with 100 μl tips, 2 million cells, and three 10 mS pulses of 1350V; each electroporation involved 7.5 μg of each of the two plasmids and 3 μg of the transposase mRNA). The transfected cells were placed immediately into RPMI medium supplemented with 10% fetal bovine serum and 200 nM methotrexate. After a week of culture, the cells were analyzed by flow cytometry for expression of BFP and RFP.

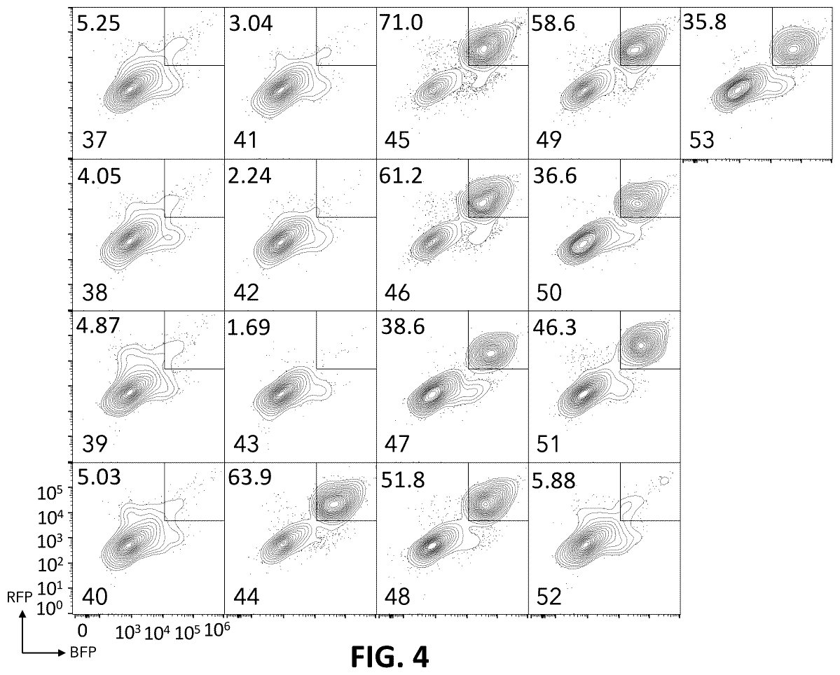

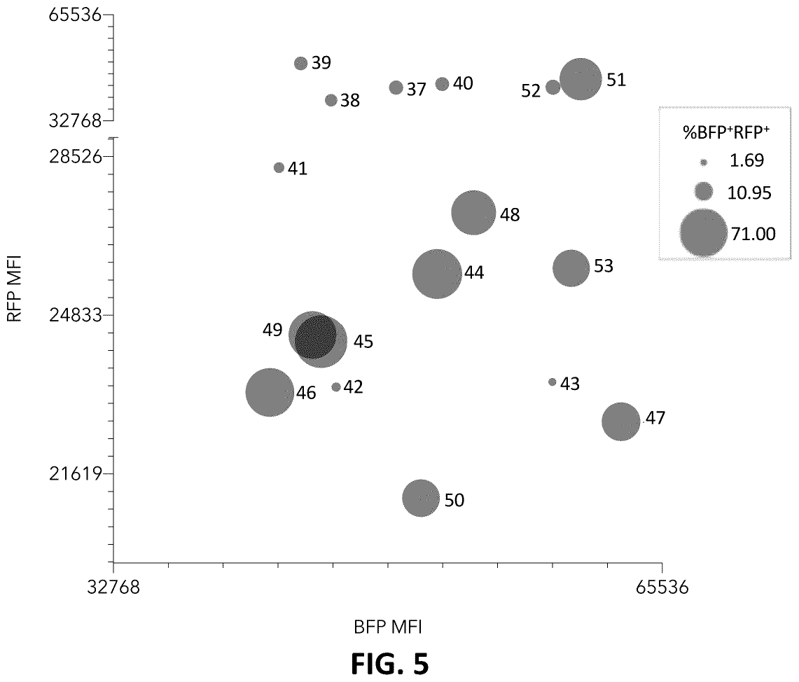

As shown in FIGS. 4 and 5, some of the carboxy-terminal fragments failed to result in substantial numbers of cells that were positive for expression of both BFP and RFP. The cultures generated with such fragments (e.g., variant nos. 37-43 (SEQ ID NOs: 123-129)) showed low numbers of viable cells at the time of analysis (FIG. 5). By contrast, substantial numbers of BFP+RFP+ cells were present in the cultures generated with carboxy-terminal variant nos. 44-51 and 53 (SEQ ID NOs: 130-137 and 139). These results suggest that mouse substitutions toward the carboxy-terminus of the carboxy-terminal fragment were important for creating a split form of DHFR when used with an amino-terminal fragment that was entirely mouse in protein sequence.

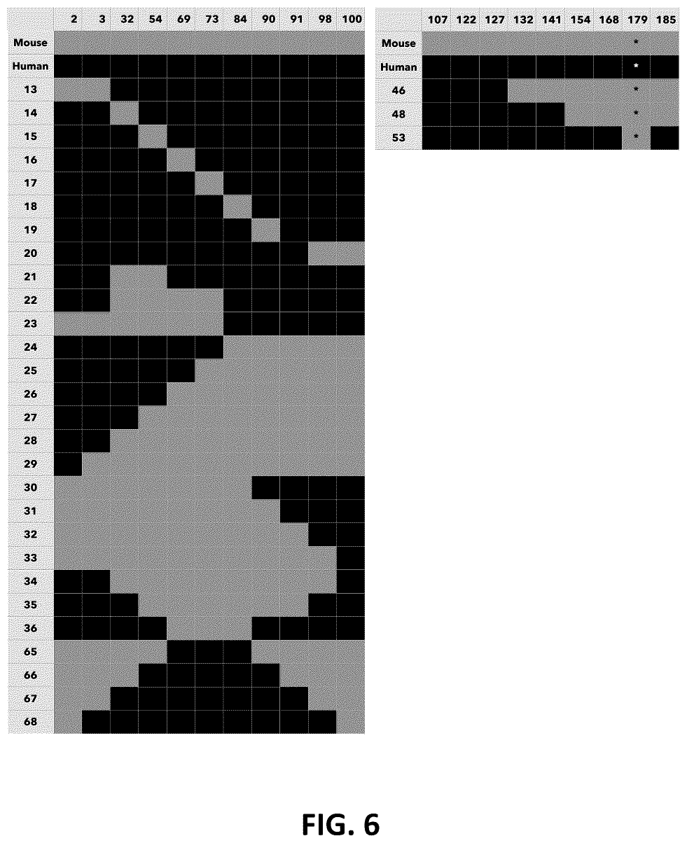

The experimental approach just described was repeated to identify mouse substitutions that would allow for an otherwise human amino-terminal fragment of DHFR to function in a split context. FIG. 6 shows a collection of chimeras (variant nos. 13-36, 65-68; SEQ ID NOs: 140-167) generated for this purpose. Transposon methodology was used as above to express these chimeras in Jurkat cells together with the three carboxy-terminal fragments shown at the right-hand side of the figure, i.e., the full collection of amino-terminal fragments was paired with carboxy-terminal variant no. 46 (SEQ ID NO: 132) in one series of transfections, with carboxy-terminal variant no. 48 (SEQ ID NO: 134) in a separate series, and finally with carboxy-terminal variant no. 53 (SEQ ID NO: 139) in an additional series. The Lonza nucleofector was used for these transfections (with SE buffer and supplement, 1 μg of each of two plasmids and 0.2 μg of Leap-In mRNA per transfection, 16×20 μl electroporation assemblies, and the manufacturer's recommended protocol). Transfected cells were cultured in RPMI medium supplemented with 10% fetal bovine serum and 200 nM methotrexate. After a week of culture, the cells were analyzed by flow cytometry for expression of BFP and RFP.

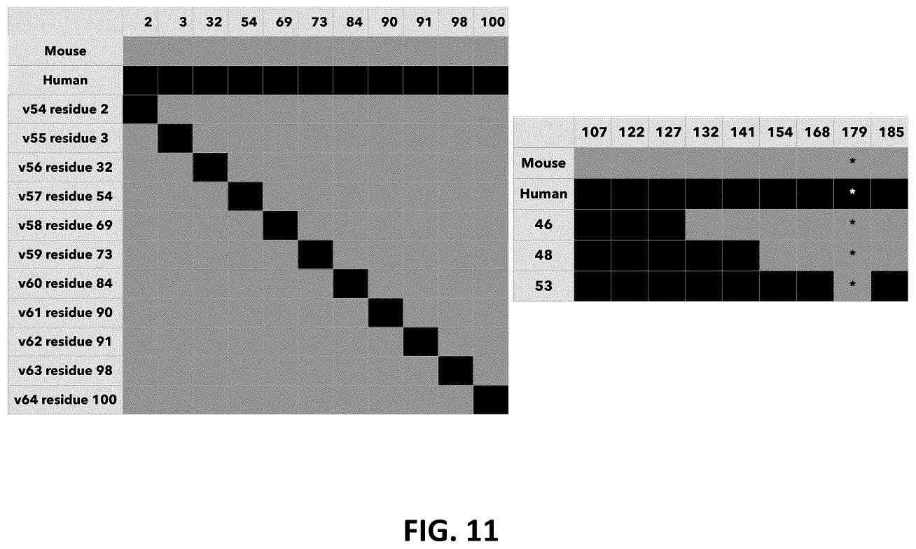

An additional series (variant nos. 54-64; SEQ ID NOs: 168-178) of amino-terminal fragments was also generated and tested as part of the above experiment. This series is shown in FIG. 11 and is comprised of chimeras in which only one human substitution was made in an otherwise fully human context. The collection of chimeras shown in FIG. 11 was likewise paired with carboxy-terminal variant no. 46 in one series of transfections, with carboxy-terminal variant no. 48 in a separate series, and finally with carboxy-terminal variant no. 53 in an additional series.

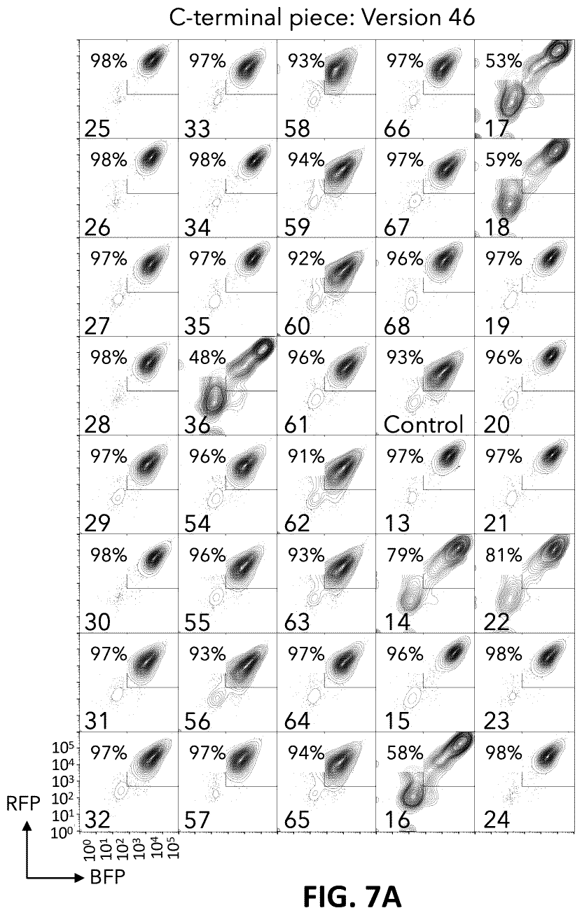

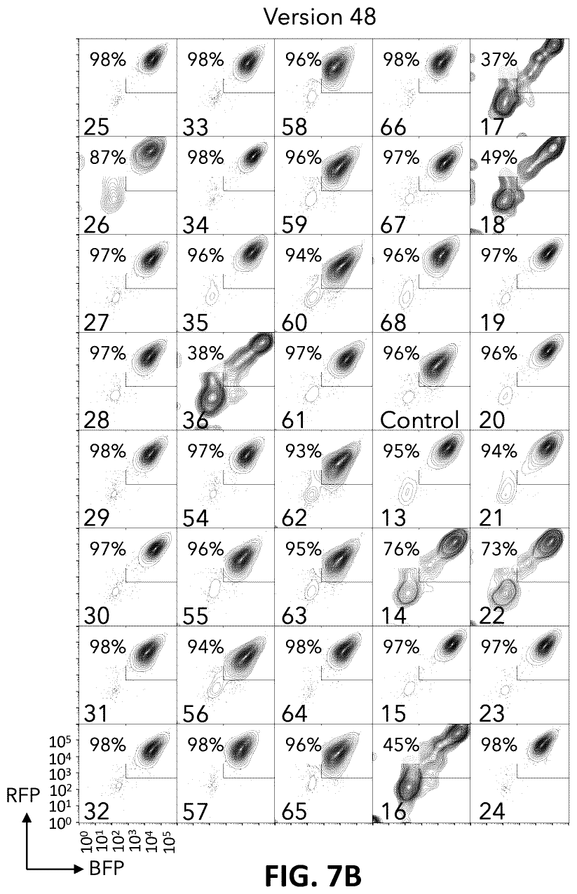

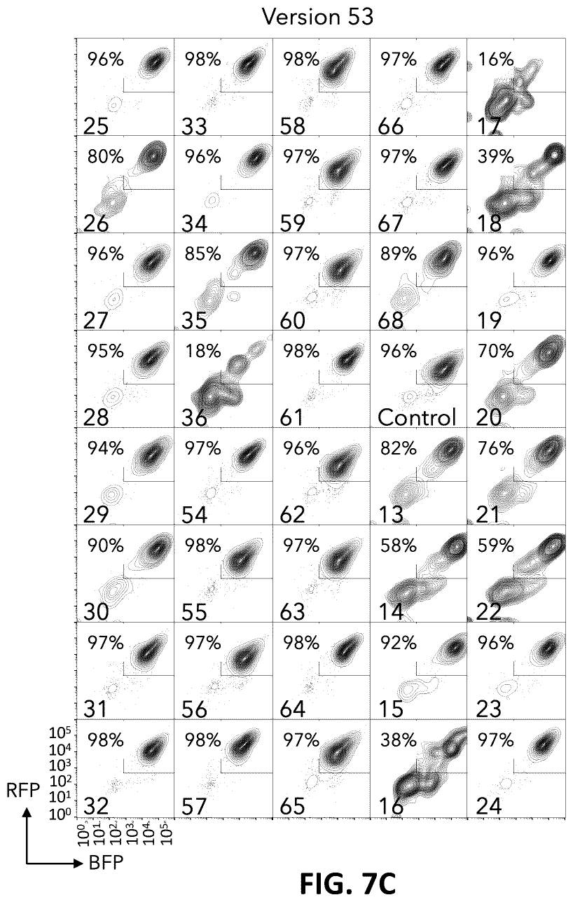

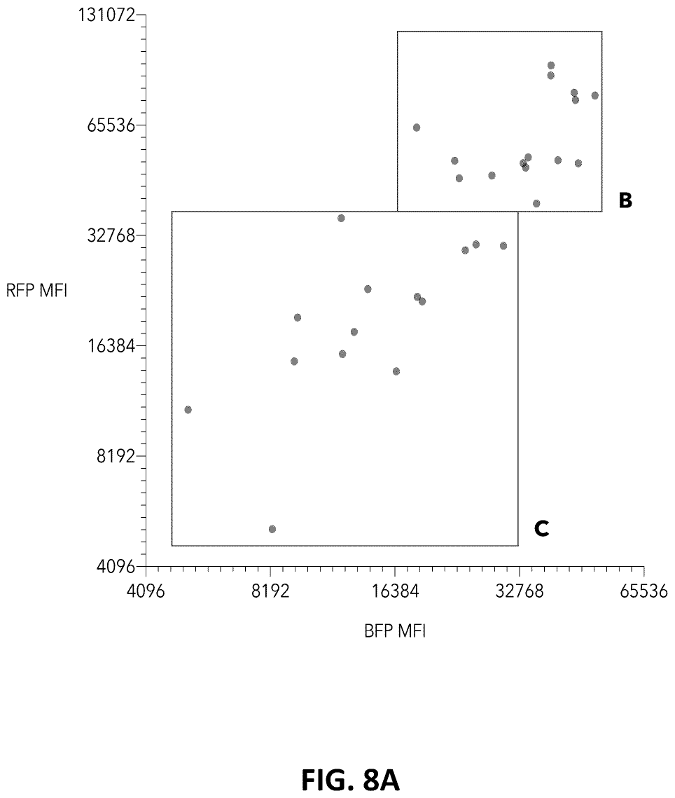

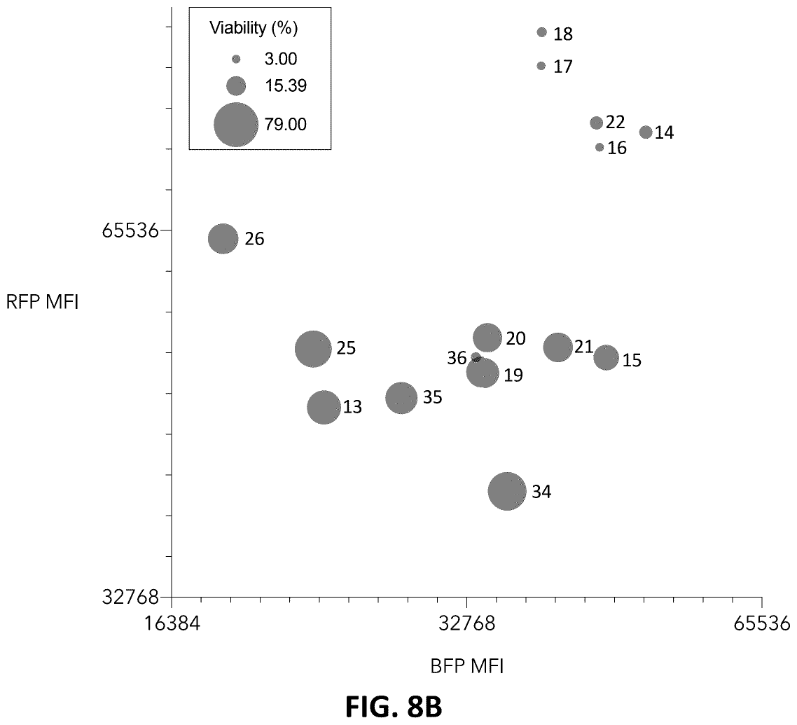

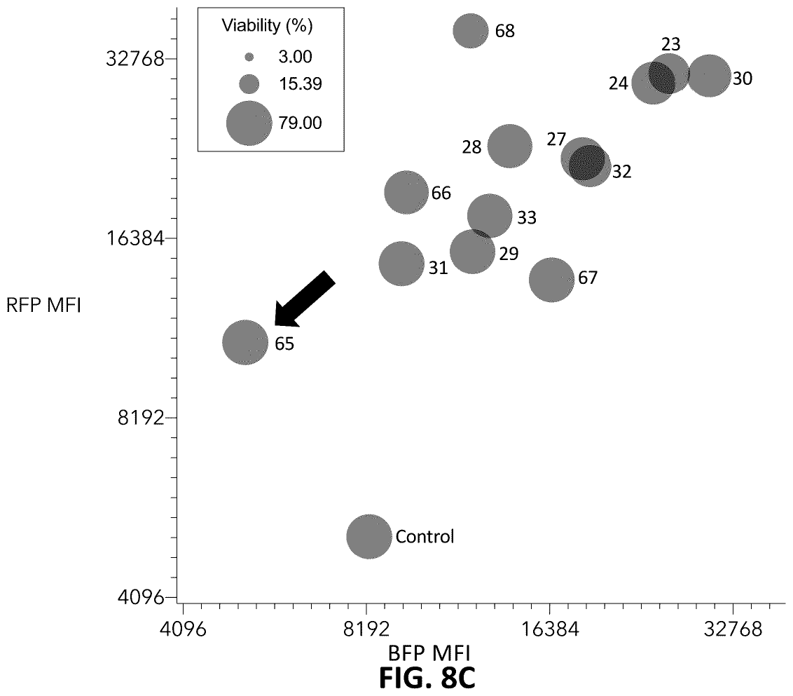

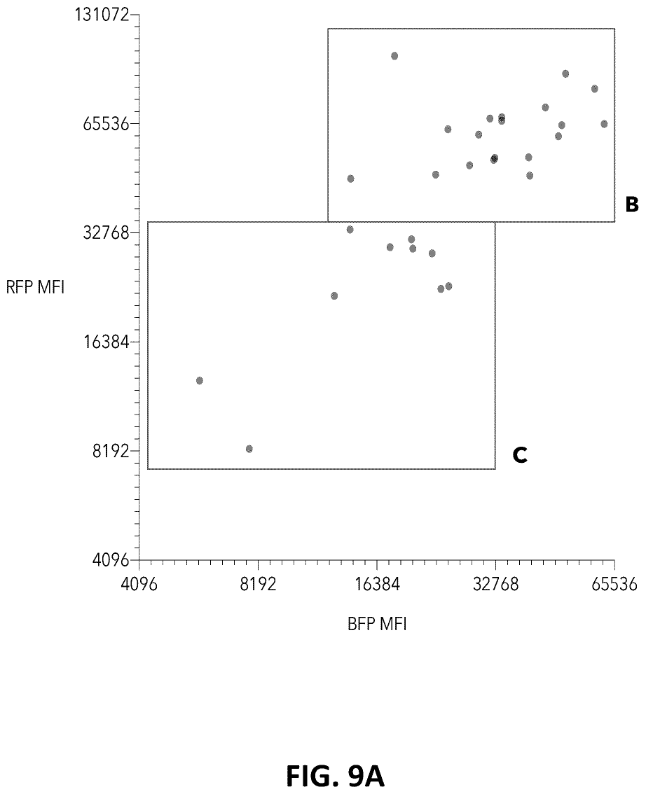

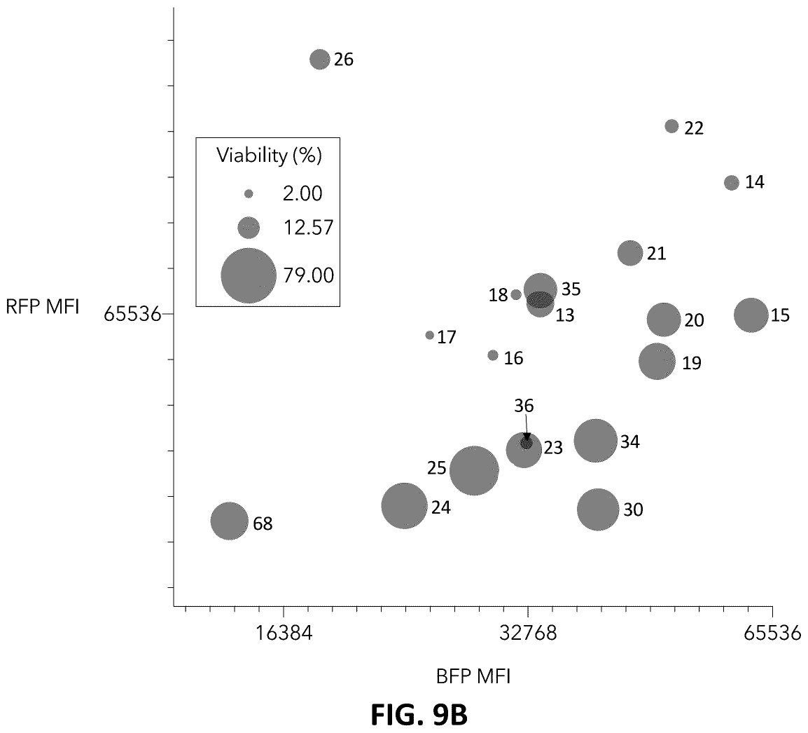

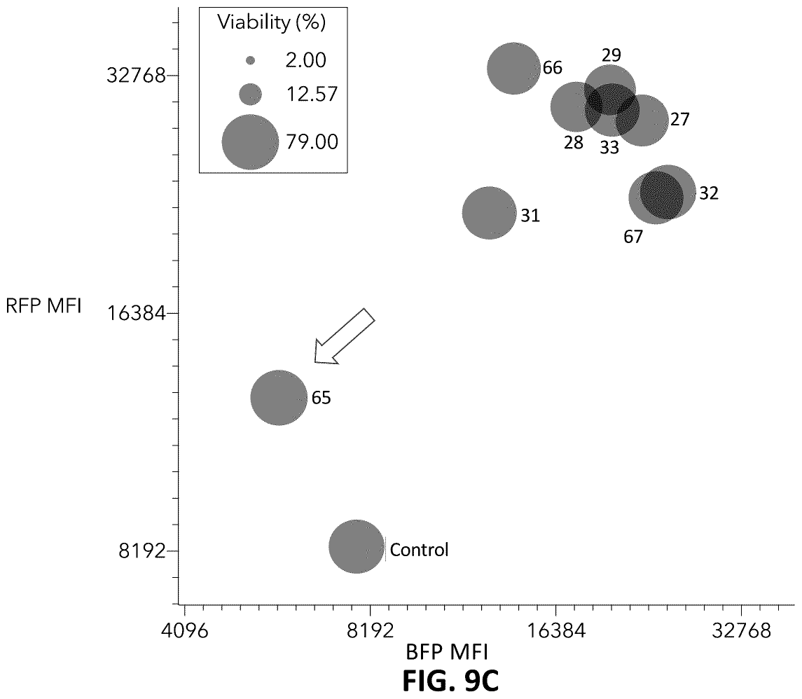

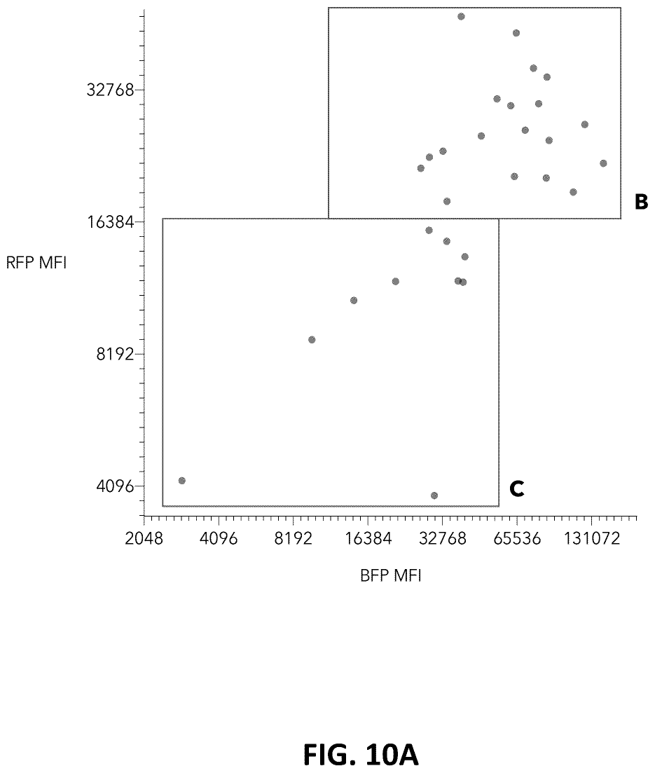

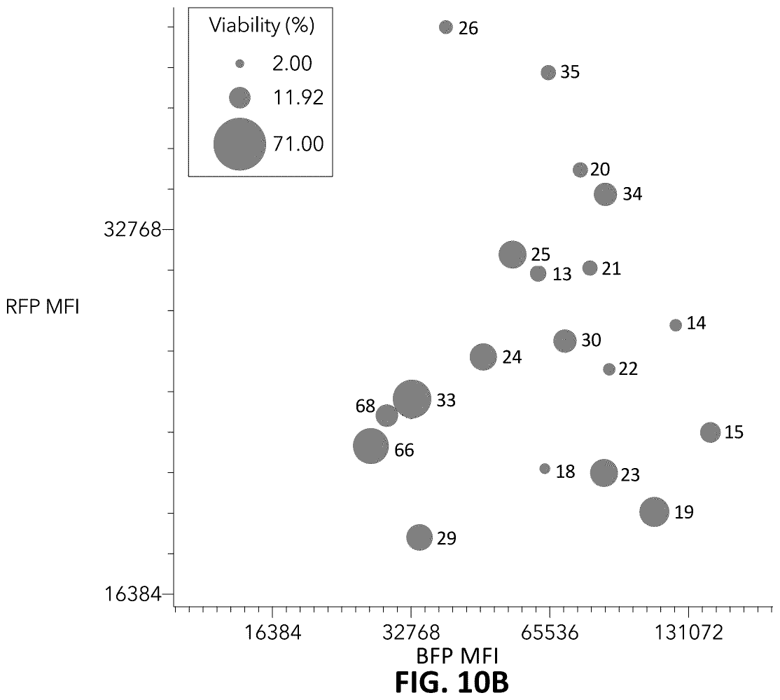

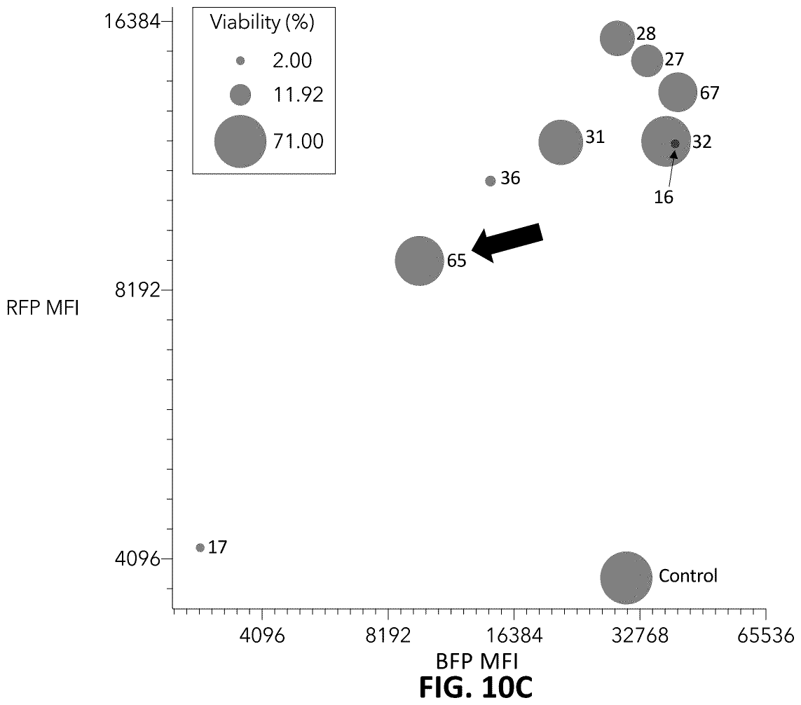

The flow cytometry data from the above experiment involving chimeras depicted in FIGS. 6 and 11 are shown in FIGS. 7A-7C. FIGS. 8A-8C, 9A-9C, and 10A-10C summarize these flow cytometry data for the amino-terminal chimeras in FIG. 6 (combined with carboxy-terminal fragment variant nos. 46, 48, and 53, respectively). Preferred combinations of chimeras yield high frequencies of BFP+RFP+ cells (FIGS. 7A-7C) with low MFIs for both reporters and good viability (FIGS. 8A-8C, 9A-9C, and 10A-10C). Chimera variant no. 65 (SEQ ID NO: 164) was associated with this phenotype when it was used with all three carboxy-terminal fragment variant nos. 46, 48, and 53. Chimera variant no. 65 was comprised of eight mouse substitutions (at positions 2, 3, 32, 54, 90, 91, 98, and 100).

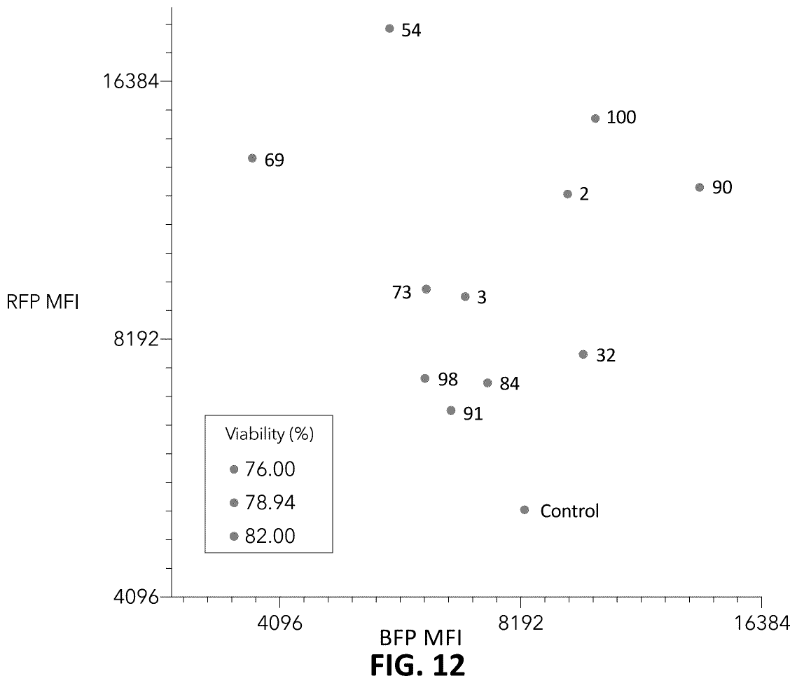

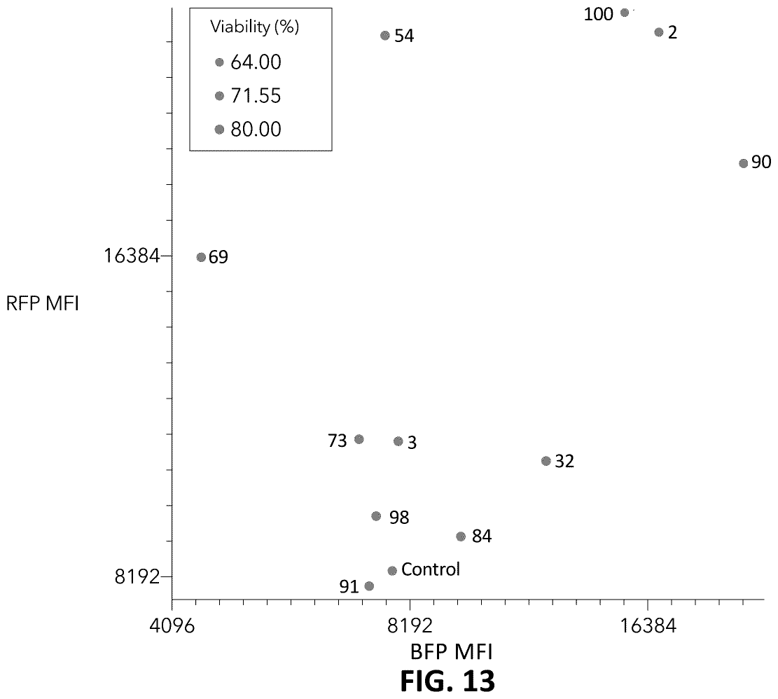

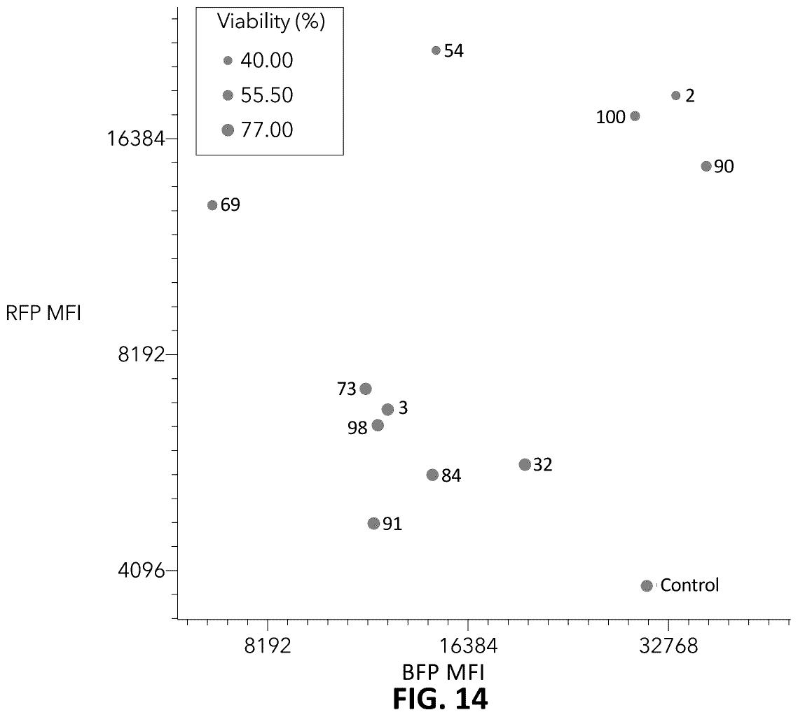

FIGS. 12-14 summarize the flow cytometry data obtained with the chimeras depicted in FIG. 11. High MFIs in these figures discriminate chimeras associated with (presumptively) relatively weak methotrexate resistance (incurring a selection for high transposon copy number). Since these chimeras each carry only a single mouse-to-human substitution in an otherwise fully mouse context, the results immediately identify substitutions that might be relatively more problematic than others (when assayed in isolation). These residues are 2, 54, 69, 90, and 100. Such data suggest that for optimal function, a split DHFR system may allow human residues at positions 3, 32, 73, 84, 91, and 98, but may require mouse residues at positions 2, 54, 69, 90, and 100.

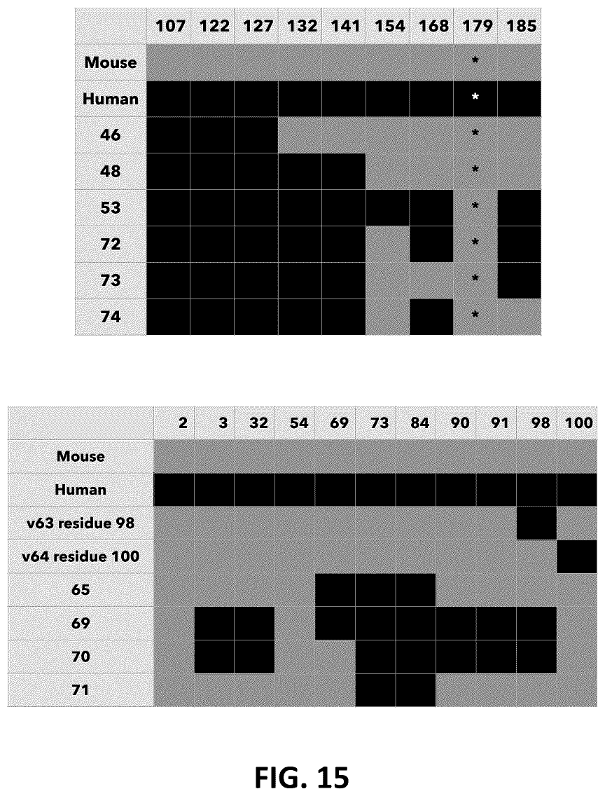

A further series of chimeras-variant nos. 69-74 (SEQ ID NOs: 179-184) depicted in FIG. 15—was generated based on the results just summarized. This series explored further which of the implicated amino- and carboxy-terminal mouse substitutions were necessary to generate a pair of chimeric fragments that functioned optimally in terms of conferring resistance to methotrexate.

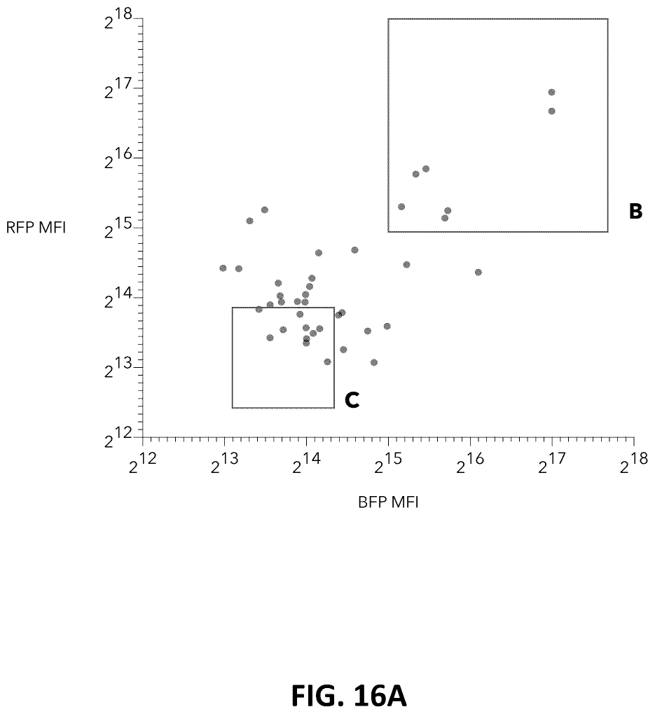

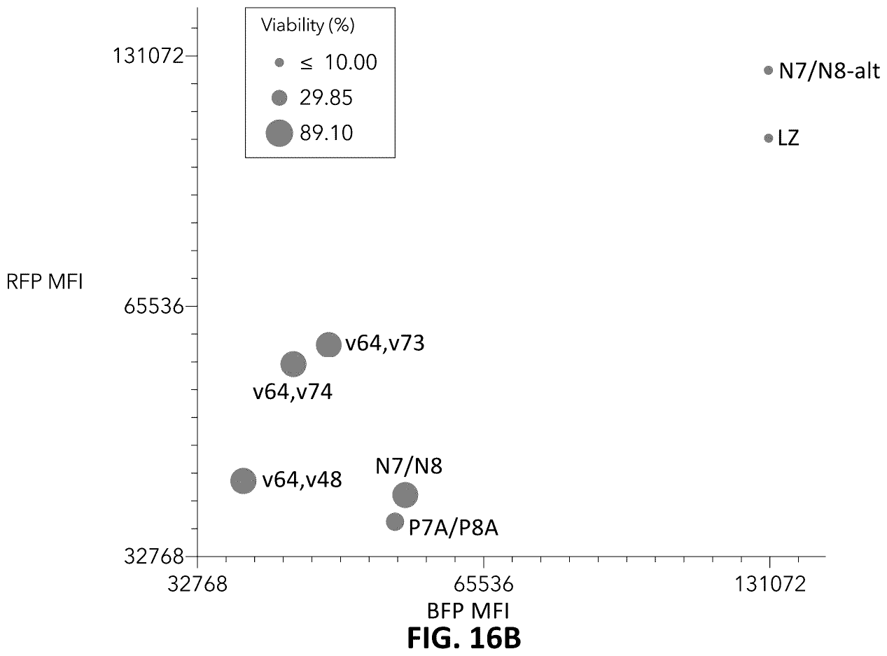

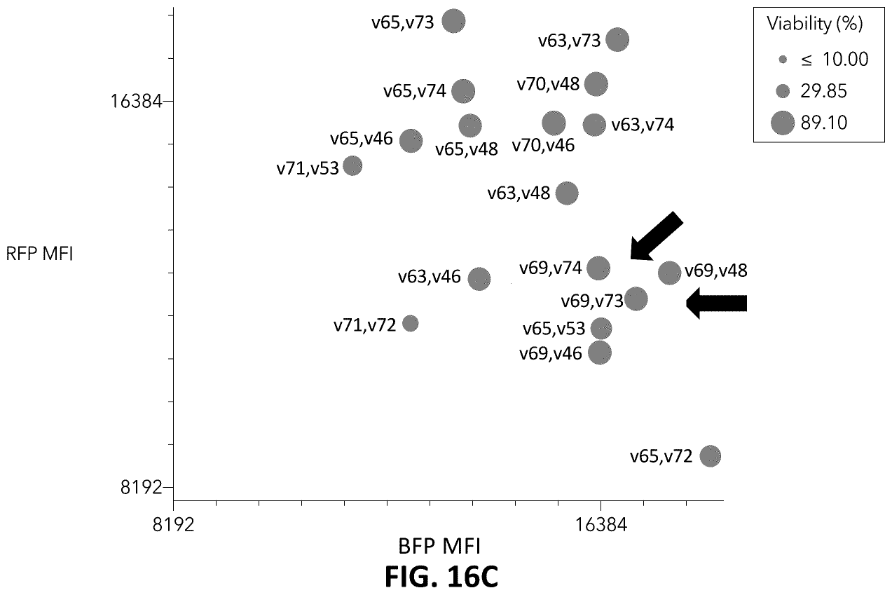

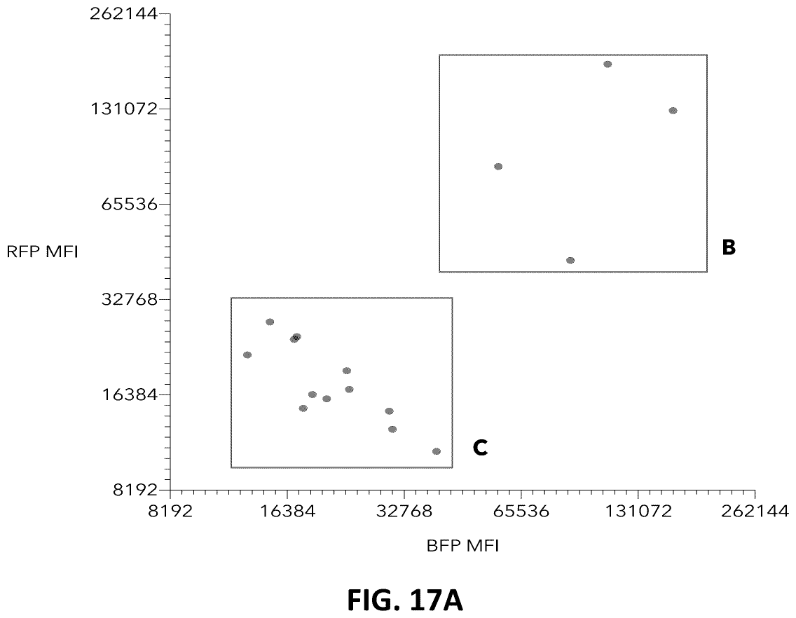

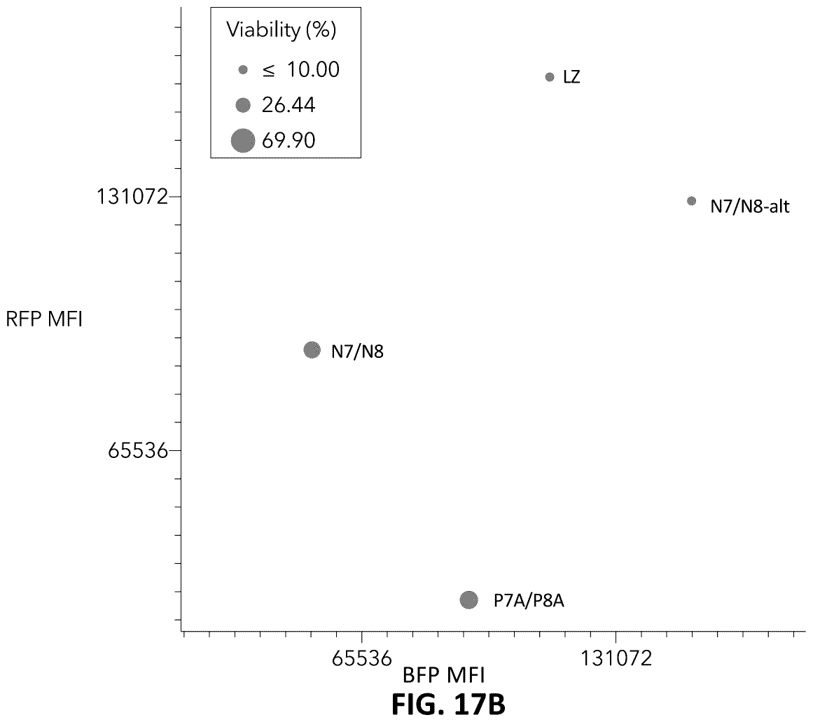

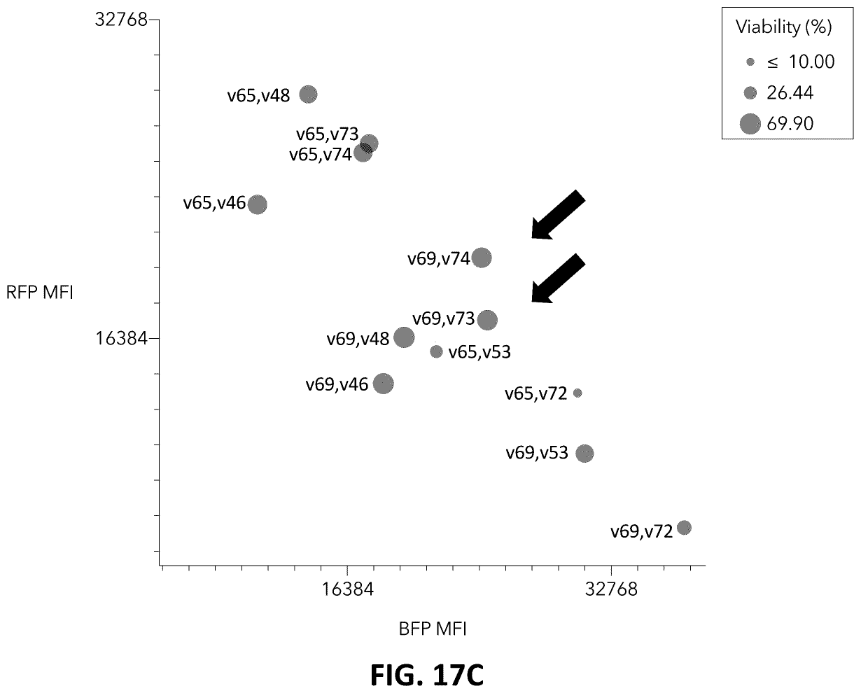

Transfections into Jurkat cells were performed (using Lonza nucleofection) as above and the transfected cells were again placed into methotrexate selection for a week prior to analysis by flow cytometry. The experiment was repeated three times with similar outcomes. Two of these outcomes are summarized in FIGS. 16A-16C and 17A-17C. The arrows in the two figures show that amino-terminal variant 69 is associated with good functionality when combined with carboxy-terminal variants 73 or 74. Importantly, these combinations of fragments perform at a similar level to the best-performing fragments comprised of a greater content of mouse residues. They also outperform the chimeras comprised entirely of mouse DHFR sequences.

The results just summarized show that mouse residues must be used at positions 2 (G2R), 54 (K54R), 73 (L73I), 100 (T100I), and either 168 (D168E) or 185 (N185K) to create a human-mouse chimeric split DHFR system. All other residues can be human (with the exception of the L22F and F31S substitutions required for methotrexate resistance).

### Example 3: Use of a Split DHFR Selection System in Human T Cells

The results summarized in the previous example involved the use of DNA constructs designed solely for the purpose of optimizing DHFR fragments for use in a selection system. To validate the outcome of the optimization experiments, a series of constructs was generated for use in Jurkat and primary T cells. Specifically, the constructs exploited the amino-terminal fragment present in variant no. 65 (i.e., with mutations to the human DHFR sequence of G2R, S3P, R32K, K54R, S90A, R91K, K98R, and T100I) and the carboxy-terminal fragment present in variant no. 48 (see FIGS. 6, 7A-7C, 8A-8C, 9A-9C, and 10A-10C) (i.e., with mutations to the human DHFR sequence of E154G, D168E, and N185K). This combination of fragments was associated with roughly equivalent performance to that of the optimized fragments comprised of a minimal content of mouse substitutions (see FIGS. 15, 16A-16C, and 17A-17C).

The construct series used for this example involved a single transposon-containing plasmid of ˜ 10 Kb in size harboring a transgene expressing the carboxy-terminal fragment DHFR. Of three other transgenes present in the transposon, one employed a constitutive house-keeping gene promoter to express a firefly luciferase protein (a red-shifted variant of the luciferase from Luciola italica). The other transgenes employed synthetic promoters previously verified as being induced in a STAT3- or NFAT-responsive manner upstream of open reading frames encoding secreted marine luciferases from Gaussia princeps or Cypridina noctiluca, respectively.

Five transposon-containing plasmids, also each ˜10 Kb in size, were generated carrying a common transgene encoding the amino-terminal fragment of DHFR (variant no. 65). Of two other transgenes present in these plasmids, one encoded a chimeric antigen receptor (the BB2121 CAR specific for BCMA, the Tisagenlecleucel CAR specific for CD19, or the 14g2a CAR specific for the ganglioside GD2), and the other encoded a variant of CD360, which is the human alpha chain from the receptor for the cytokine IL-21.

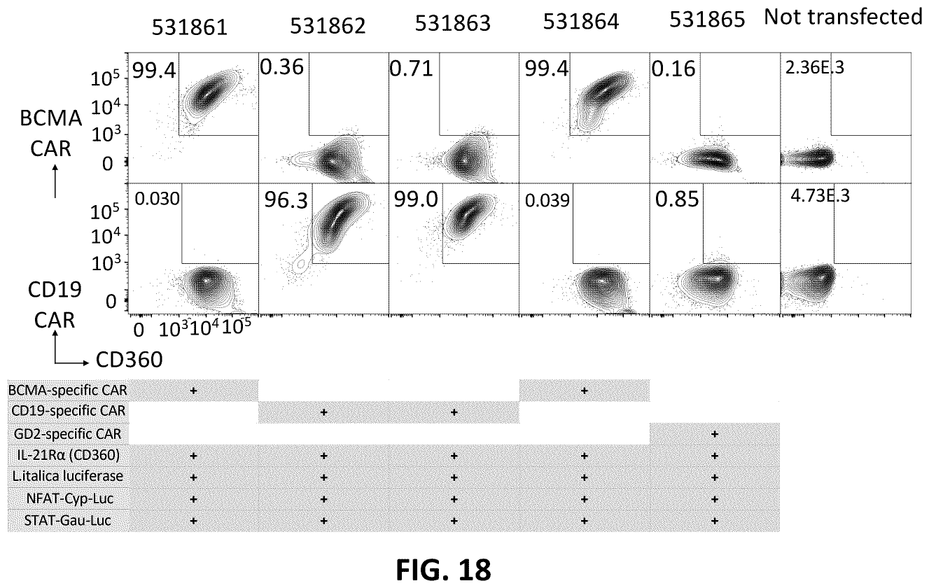

As an initial test of these constructs, they were co-transfected into Jurkat cells using the Lonza Nucleofector instrument (with SE buffer and supplements according to the manufacturer's recommended procedure). The transfected cells were plated immediately in RPMI-1640 medium supplemented with 10% (vol/vol) fetal bovine serum and methotrexate at 200 nM. After more than two weeks of selection, the cells were analyzed by flow cytometry to confirm expression of both the BB2121 and Tisagenlecleucel CARs and CD360 (FIG. 18). Expression of the GD2-specific CAR was not assessed by flow cytometry but was confirmed functionally (see below).

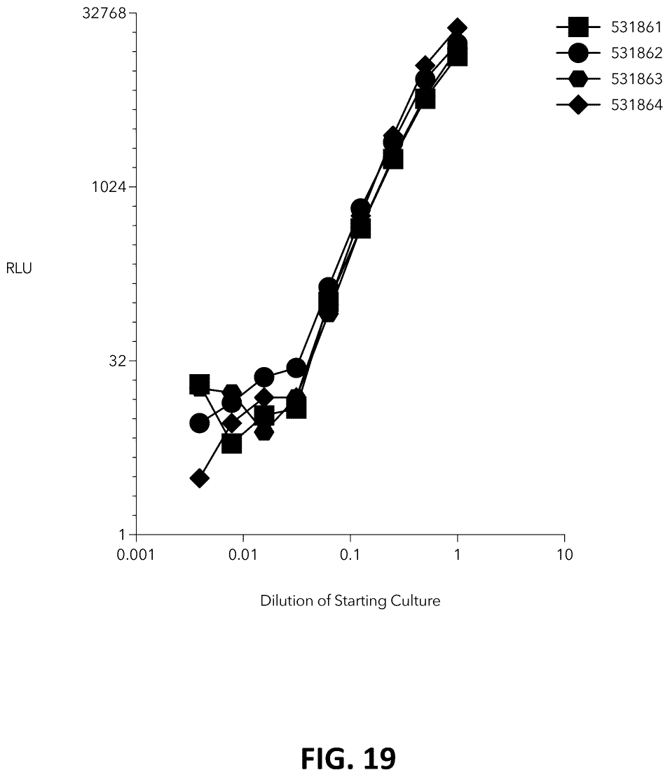

To confirm that the firefly luciferase transgene had been transferred into the transfected cells, aliquots of four of the cultures were serially diluted eight times (a two-fold dilution each time) and assayed by luminometry after the application of a luciferin-containing buffer (FLAR from Targeting Systems, El Cajon, CA, USA). As shown in FIG. 19, titratable luciferase activity was present in all of the cultures.

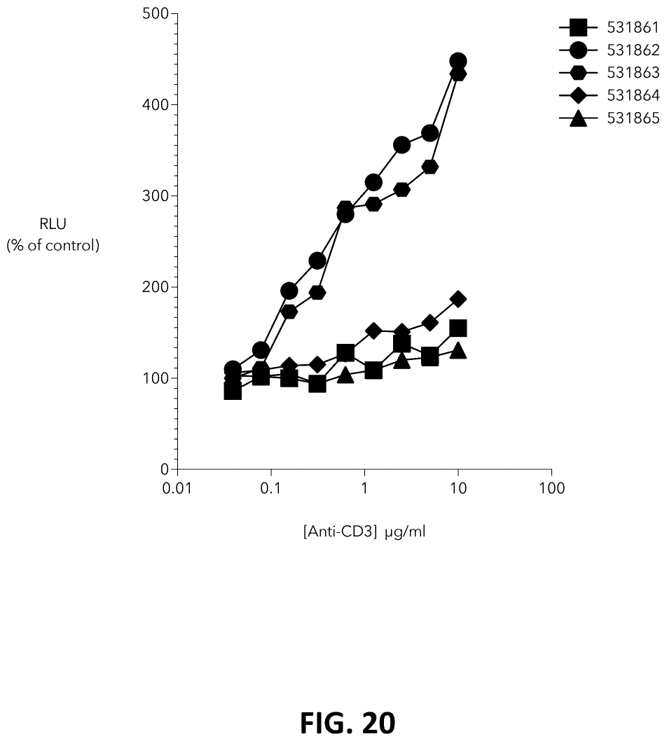

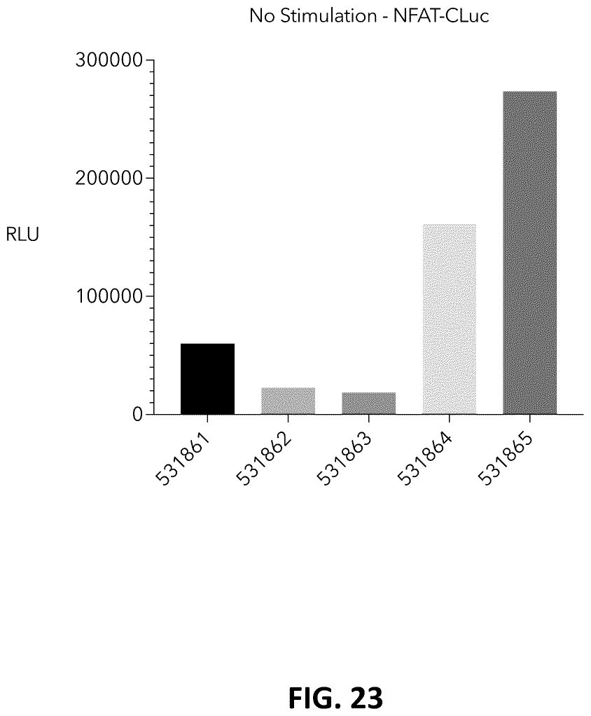

Two approaches were taken to confirm that the NFAT-luciferase transgene (expressing the marine luciferase from Cypridina noctiluca) was functional in the methotrexate-selected cell cultures. One was to subject the cells to a titration of anti-CD3 monoclonal antibody, which induces signaling through the TCR/CD3 complex and thereby causes NFAT-dependent induction of C. noctiluca luciferase. As shown in FIG. 20, this treatment successfully resulted in titratable induction of luciferase in all five of the cell cultures. Notably, the extent of induction was higher in cells expressing the CD19-specific CAR than in those expressing either the BCMA- or GD2-specific CARs. This difference could be readily explained by variance in the amount of basal expression of the reporter in the absence of anti-CD3 stimulation (see FIG. 23), this being higher for the BCMA- and GD2-specific CARs than for the CD19-specific CAR, consistent with the propensity of these CARs to induce so-called tonic signaling associated with spontaneous aggregation of the CARs on the cell surface.

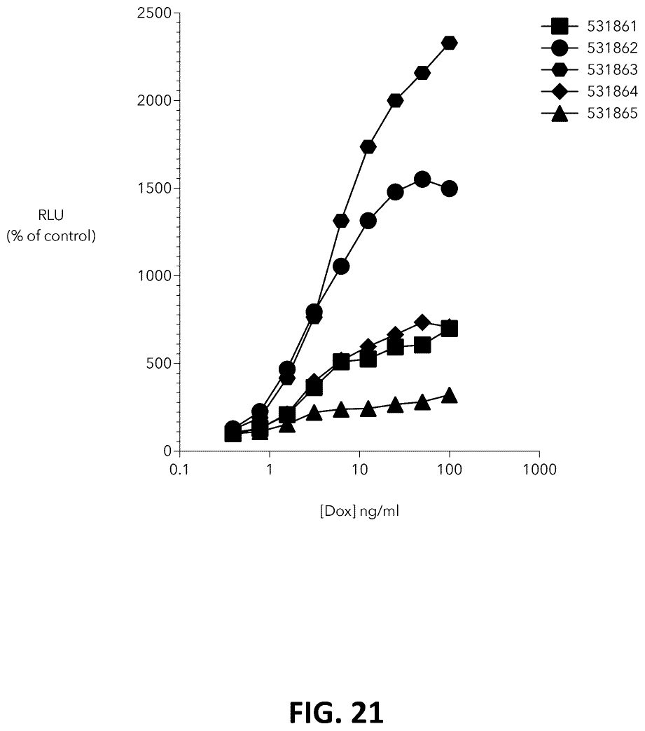

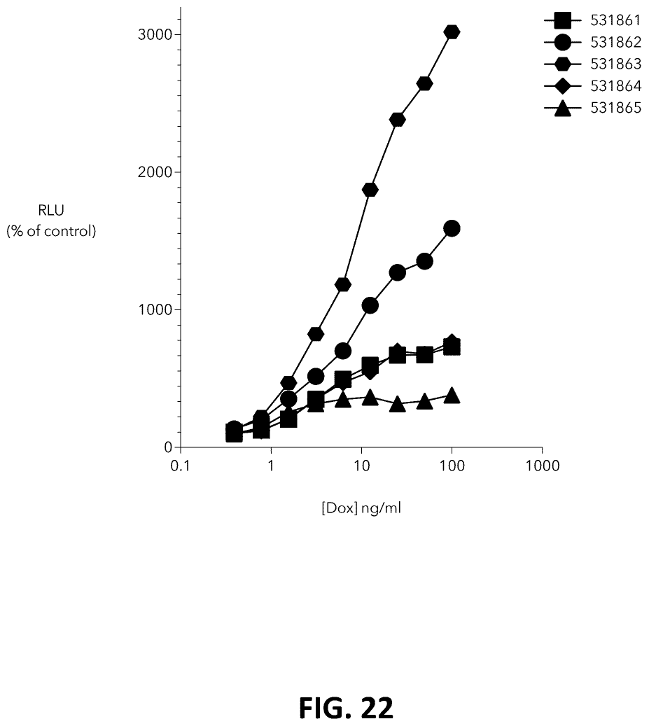

The second approach for assessing the functionality of the NFAT-luciferase transgene in the transfected cells was to expose them to target cells that differed in their relative expression of the antigens recognized by the CARs the Jurkat cells expressed. This was accomplished through use of target cells (mouse EL-4 thymoma cells) that expressed the antigens in a tetracycline/doxycycline-regulated fashion. The target cells were exposed to a titration of doxycycline for two days before mixture with the transfected Jurkat cells for a further overnight period prior to assaying secreted luciferase in the culture medium by luminometry (using the VLAR-2 reagent and Vargulin from Targeting Systems, El Cajon, CA, USA). Two different clones of EL4 target cells were used in parallel in each case, with the results obtained shown in FIGS. 21 and 22. Here again, the extent of induction of luciferase was impacted by the basal level of luciferase expression in the cells, with the CD19-specific cells (characterized by low CAR tonic signaling) showing the greatest reporter induction.

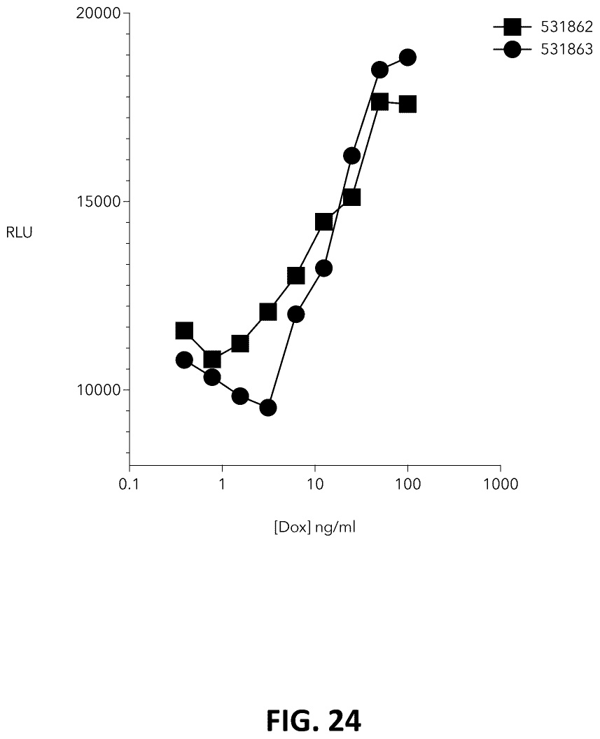

Functionality of the STAT3-luciferase transgene in the cells was also assessed by sampling supernatant fluid from the stimulated cells just described. In this case, the Gaussia princeps luciferase required coelenterazine as a substrate and was assayed using the GAR reagent and substrate from Targeting Systems (El Cajon, CA, USA). As shown in FIG. 24 for the CD19-specific cells, antigen-dependent reporter induction could be readily detected.

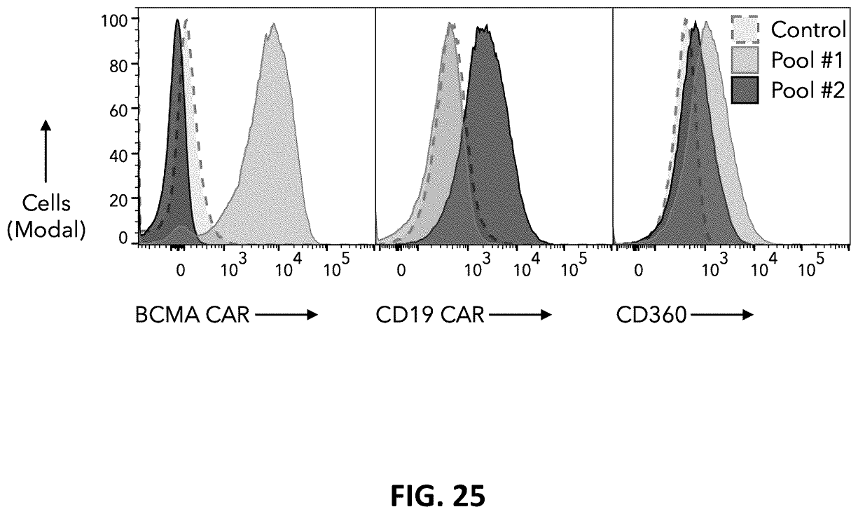

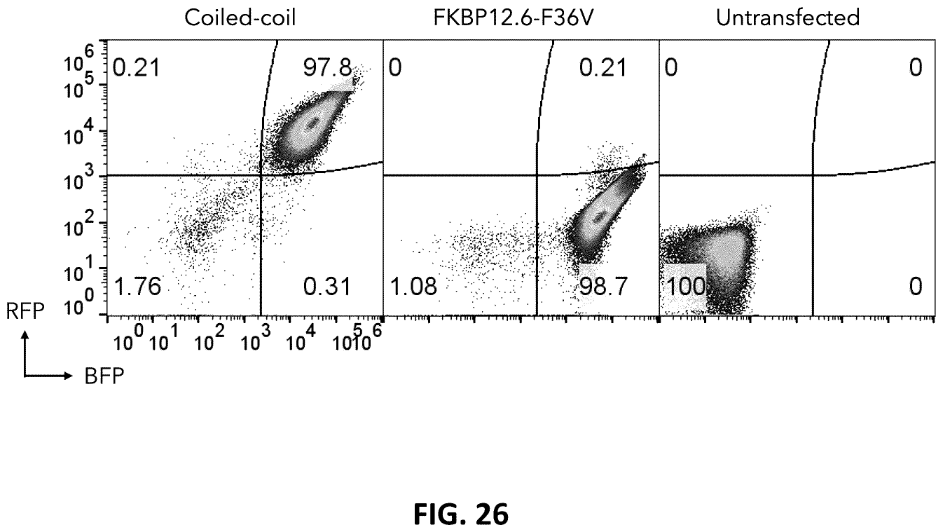

Finally, the split DHFR constructs based on variant nos. 65 and 48 were tested for functionality in primary T cells. In this case, two constructs that expressed the amino-terminal fragment of DHFR (variant no. 65) were selected for testing: one carrying the BCMA-specific CAR transgene and the other the CD19-specific CAR transgene. In both cases, a CD360 transgene was also present on the construct. Constructs of ˜6 Kb carrying a transgene encoding the carboxy-terminal fragment of DHFR (variant no. 48) were combined with the CAR constructs, and the cells were selected in 200 nM methotrexate prior to analysis by flow cytometry for CAR and CD360 expression. The results shown in FIG. 25 confirm the expected reciprocal pattern of CAR expression in the derived cell pools consistent with functionality of both the CAR and CD360 transgenes.

### Example 4: Split DHFR Complementation Assay to Show Rimiducid-Dependent Dimerization

The Split DHFR concept was employed as the basis of a test of whether a fragment of the human protein FKBP12.6 could dimerize in the presence of rimiducid. For this purpose, an F36V substitution was introduced into FKBP12.6 in an effort to render it sensitive to rimiducid-dependent dimerization as is the case for the paralogous protein FKBP12. See Clackson T, Yang W, Rozamus L W, Hatada M, Amara J F, Rollins C T, Stevenson L F, Magari S R, Wood S A, Courage N L, Lu X, Cerasoli F, Gilman M, Holt D A. Redesigning an FKBP-ligand interface to generate chemical dimerizers with novel specificity. Proc Natl Acad Sci USA. 1998; 95 (18): 10437-42. doi: 10.1073/pnas.95.18.10437.

The truncated version of FKBP12.6 carrying the F36V substitution (SEQ ID NO: 192) was fused at its carboxy terminus to the two fragments of murine DHFR as in Example 1, with one of the fragments bearing the L22F and F31S substitutions to confer methotrexate resistance (SEQ ID NOs: 190 and 191).

As a control, the P7A/P8A pair of DHFR fragment fusion proteins (involving SEQ ID Nos: 188 and 189) was also used. Whereas the P7A/P8A pair of constructs co-expressed the DHFR fragments with a red fluorescent protein (RFP; plobRFP) or a blue fluorescent protein (BFP; mTagBFP2), both FKBP12.6 tr transposons carried a BFP transgene.

Jurkat cells were co-transfected by electroporation (ThermoFisher Neon; 100 μl tips; 1350V, three 10 ms pulses) with pairs of plasmids carrying transposons encoding the two DHFR fragments (7.5 μg of each) together with mRNA encoding the Leap-In Transposase® enzyme (2 μg). The cells were plated in RPMI-1640 medium supplemented with fetal bovine serum (10% vol/vol) and methotrexate (0.2 μM). Rimiducid at 100 nM was added to the medium of cells that had been transfected with the FKBP12.6tr pair of plasmids.

FIG. 2 shows flow cytometry data acquired after the transfected cells had been selected in methotrexate for two weeks. At left is the result for cells expressing the DHFR fragments fused to the P7A/P8A heterodimerizing coiled-coil peptides. In this case, one transposon carried an RFP gene, while the other had a BFP gene, and the majority of surviving cells co-expressed both fluorescent proteins. In the center is the result obtained when the fusions were both to the FKBP12.6tr_F36V protein and the cells were cultured in the presence of rimiducid. Both transposons in this case carried a BFP gene. Untransfected cells were analyzed under identical conditions as a control (at right).

The control P7A/P8A coiled coil fusion proteins conferred methotrexate resistance on the transfected cells and permitted the outgrowth of cells that were predominantly double-positive for BFP and RFP. Similarly, the FKBP12.6tr_F36V-based fusion proteins also allowed survival in the presence of methotrexate, though in this case, the cells that grew out were only BFP+ (because RFP was not used, and instead, both transposons carried an mTagBFP2 gene). Additional experiments showed that this latter survival required rimiducid because the cells failed to thrive when the drug was removed.

1. A method for selecting cells that have stably acquired one or more genes of interest, the method comprising:
(A) co-transfecting a plurality of cells with:
(1) a first heterologous polynucleotide comprising a nucleotide sequence that encodes a first fusion protein, the first fusion protein comprising:
(a) a first dimerization domain; and
(b) a first mutant human enzyme dihydrofolate reductase (hDHFR) fragment comprising amino acid substitutions, numbered relative to SEQ ID NO: 3, of L22F, F31S, G2R, K54R, and T100I; and
(2) a second heterologous polynucleotide comprising a nucleotide sequence that encodes a second fusion protein, the second fusion protein comprising:
(a) a second dimerization domain that co-associates with the first dimerization domain when expressed in a cell; and
(b) a second mutant hDHFR fragment comprising amino acid substitutions, numbered relative to SEQ ID NO: 3, of at least one of D168E and N185K,
wherein, but for the mutations to the first and second mutant hDHFR fragments, the first and second mutant hDHFR fragments are normally contiguous to each other, their breakpoint occurring in the immediate vicinity of the surface exposed loop comprised of residues 101-108, numbered relative to SEQ ID NO: 3;
wherein the first and second mutant hDHFR fragments are catalytically inactive in isolation or when co-expressed in cells, but when brought into proximity with one another by fusion to the first and second dimerization domains, respectively, demonstrate resistance to methotrexate; and
wherein at least one of the first heterologous polynucleotide and the second heterologous polynucleotide further comprise a nucleotide sequence that encodes a gene of interest; and
(B) subjecting the cells to methotrexate.

2. The method of claim 1, wherein the first mutant hDHFR fragment further comprises an amino acid substitution, numbered relative to SEQ ID NO: 3, of L73I.

3. The method of claim 1, wherein the first mutant hDHFR fragment further comprises amino acid substitutions, numbered relative to SEQ ID NO: 3, of S3P, R32K, S90A, R91K, and K98R.

4. The method of claim 1, wherein the second mutant hDHFR fragment further comprises an amino acid substitution, numbered relative to SEQ ID NO: 3, of E154G.

5. The method of claim 4, wherein the second mutant hDHFR fragment further comprises amino acid substitutions, numbered relative to SEQ ID NO: 3, of K132R and D141E.

6. The method of claim 1, wherein the first dimerization domain and the second dimerization domain each independently comprise a GCN4 leucine zipper comprising SEQ ID NO: 254.

7. The method of claim 1, wherein one of the first dimerization domain and the second dimerization domain comprises the N7 hetero-dimerizing synthetic coiled coil peptide comprising SEQ ID NO: 255, and the other dimerization domain comprises the N8 hetero-dimerizing synthetic coiled coil peptide comprising SEQ ID NO: 256, of the N7/N8 pair of hetero-dimerizing synthetic coiled coil peptides, respectively.

8. The method of claim 1, wherein one of the first dimerization domain and the second dimerization domain comprises the P7A hetero-dimerizing synthetic coiled coil peptide comprising SEQ ID NO: 257, and the other dimerization domain comprises the P8A hetero-dimerizing synthetic coiled coil peptide comprising SEQ ID NO: 258, of the P7A/P8A pair of hetero-dimerizing synthetic coiled coil peptides, respectively.

9. The method of claim 1, wherein the first dimerization domain and the second dimerization domain each independently comprise an FKBP-based dimerization domain, and the method further comprises subjecting the cells to a chemical inducer of dimerization.

10. The method of claim 1, wherein the first dimerization domain and the second dimerization domain each independently comprise an FKBP mutant represented by SEQ ID NO: 192, and the method further comprises subjecting the cells to a chemical inducer of dimerization.

11. The method of claim 1, wherein the first heterologous polynucleotide further comprises a nucleotide sequence that encodes for a first fluorescent protein that fluoresces in a first color in the visible region when exposed to a wavelength of light that excites its chromophore, and the second heterologous polynucleotide further comprises a nucleotide sequence that encodes for a second fluorescent protein that fluoresces in a second color in the visible region when exposed to a wavelength of light that excites its chromophore.

12. The method of claim 11, further comprising subjecting the methotrexate-treated cells to flow cytometry, fluorescent microscopy, or both.

13. A system for selecting cells that have stably acquired a gene of interest, the system comprising:
(1) a first heterologous polynucleotide comprising a nucleotide sequence that encodes a first fusion protein, the first fusion protein comprising:
(a) a first dimerization domain; and
(b) a first mutant human enzyme dihydrofolate reductase (hDHFR) fragment comprising amino acid substitutions, numbered relative to SEQ ID NO: 3, of L22F, F31S, G2R, K54R, and T100I; and
(2) a second heterologous polynucleotide comprising a nucleotide sequence that encodes a second fusion protein, the second fusion protein comprising:
(a) a second dimerization domain that co-associates with the first dimerization domain when expressed in a cell; and
(b) a second mutant hDHFR fragment comprising amino acid substitutions, numbered relative to SEQ ID NO: 3, of at least one of D168E and N185K,
wherein, but for the mutations to the first and second mutant hDHFR fragments, the first and second mutant hDHFR fragments are normally contiguous to each other, their breakpoint occurring in the immediate vicinity of the surface exposed loop comprised of residues 101-108, numbered relative to SEQ ID NO: 3;
wherein the first and second mutant hDHFR fragments are catalytically inactive in isolation or when co-expressed in cells, but when brought into proximity with one another by fusion to the first and second dimerization domains, respectively, demonstrate resistance to methotrexate; and
wherein at least one of the first heterologous polynucleotide and the second heterologous polynucleotide further comprise a nucleotide sequence that encodes a gene of interest.

14. The system of claim 13, wherein the first mutant hDHFR fragment further comprises an amino acid substitution, numbered relative to SEQ ID NO: 3, of L73I.

15. The system of claim 13, wherein the first mutant hDHFR fragment further comprises amino acid substitutions, numbered relative to SEQ ID NO: 3, of S3P, R32K, S90A, R91K, and K98R.

16. The system of claim 13, wherein the second mutant hDHFR fragment further comprises an amino acid substitution, numbered relative to SEQ ID NO: 3, of E154G.

17. The system of claim 16, wherein the second mutant hDHFR fragment further comprises amino acid substitutions, numbered relative to SEQ ID NO: 3, of K132R and D141E.

18. The system of claim 13, wherein the first dimerization domain and the second dimerization domain each independently comprise a GCN4 leucine zipper comprising SEQ ID NO: 254.

19. The system of claim 13, wherein one of the first dimerization domain and the second dimerization domain comprises the N7 hetero-dimerizing synthetic coiled coil peptide comprising SEQ ID NO: 255, and the other dimerization domain comprises the N8 hetero-dimerizing synthetic coiled coil peptide comprising SEQ ID NO: 256, of the N7/N8 pair of hetero-dimerizing synthetic coiled coil peptides, respectively.

20. The system of claim 13, wherein one of the first dimerization domain and the second dimerization domain comprises the P7A hetero-dimerizing synthetic coiled coil peptide comprising SEQ ID NO: 257, and the other dimerization domain comprises the P8A hetero-dimerizing synthetic coiled coil peptide comprising SEQ ID NO: 258, of the P7A/P8A pair of hetero-dimerizing synthetic coiled coil peptides, respectively.

21. The system of claim 13, wherein the first dimerization domain and the second dimerization domain each independently comprise an FKBP-based dimerization domain.

22. The system of claim 13, wherein the first dimerization domain and the second dimerization domain each independently comprise an FKBP mutant represented by SEQ ID NO: 192.

23. The system of claim 13, wherein the first heterologous polynucleotide further comprises a nucleotide sequence that encodes for a first fluorescent protein that fluoresces in a first color in the visible region when exposed to a wavelength of light that excites its chromophore, and the second heterologous polynucleotide further comprises a nucleotide sequence that encodes for a second fluorescent protein that fluoresces in a second color in the visible region when exposed to a wavelength of light that excites its chromophore.

24. A modified cell is provided, the modified cell expressing:
(1) a gene of interest;
(2) a first heterologous polynucleotide comprising a nucleotide sequence that encodes a first fusion protein, the first fusion protein comprising:
(a) a first dimerization domain; and
(b) a first mutant human enzyme dihydrofolate reductase (hDHFR) fragment comprising amino acid substitutions, numbered relative to SEQ ID NO: 3, of L22F, F31S, G2R, K54R, and T100I; and
(3) a second heterologous polynucleotide comprising a nucleotide sequence that encodes a second fusion protein, the second fusion protein comprising:
(a) a second dimerization domain that co-associates with the first dimerization domain when expressed in a cell; and
(b) a second mutant hDHFR fragment comprising amino acid substitutions, numbered relative to SEQ ID NO: 3, of at least one of D168E and N185K,
wherein, but for the mutations to the first and second mutant hDHFR fragments, the first and second mutant hDHFR fragments are normally contiguous to each other, their breakpoint occurring in the immediate vicinity of the surface exposed loop comprised of residues 101-108, numbered relative to SEQ ID NO: 3; and
wherein the first and second mutant hDHFR fragments are catalytically inactive in isolation or when co-expressed in the cell, but when brought into proximity with one another by fusion to the first and second dimerization domains, respectively, demonstrate resistance to methotrexate.

25. The modified cell of claim 24, wherein the first mutant hDHFR fragment further comprises an amino acid substitution, numbered relative to SEQ ID NO: 3, of L73I.

26. The modified cell of claim 24, wherein the first mutant hDHFR fragment further comprises amino acid substitutions, numbered relative to SEQ ID NO: 3, of S3P, R32K, S90A, R91K, and K98R.

27. The modified cell of claim 24, wherein the second mutant hDHFR fragment further comprises an amino acid substitution, numbered relative to SEQ ID NO: 3, of E154G.

28. The modified of claim 27, wherein the second mutant hDHFR fragment further comprises amino acid substitutions, numbered relative to SEQ ID NO: 3, of K132R and D141E.

29. The modified cell of claim 24, wherein the first dimerization domain and the second dimerization domain each independently comprise a GCN4 leucine zipper comprising SEQ ID NO: 254.

30. The modified cell of claim 24, wherein one of the first dimerization domain and the second dimerization domain comprises the N7 hetero-dimerizing synthetic coiled coil peptide comprising SEQ ID NO: 255, and the other dimerization domain comprises the N8 hetero-dimerizing synthetic coiled coil peptide comprising SEQ ID NO: 256, of the N7/N8 pair of hetero-dimerizing synthetic coiled coil peptides, respectively.

31. The modified cell of claim 24, wherein one of the first dimerization domain and the second dimerization domain comprises the P7A hetero-dimerizing synthetic coiled coil peptide comprising SEQ ID NO: 257, and the other dimerization domain comprises the P8A hetero-dimerizing synthetic coiled coil peptide comprising SEQ ID NO: 258, of the P7A/P8A pair of hetero-dimerizing synthetic coiled coil peptides, respectively.

32. The modified cell of claim 24, wherein the first dimerization domain and the second dimerization domain each independently comprise an FKBP-based dimerization domain.

33. The modified cell of claim 24, wherein the first dimerization domain and the second dimerization domain each independently comprise an FKBP mutant represented by SEQ ID NO: 192.

34. The modified cell of claim 24, wherein the first heterologous polynucleotide further comprises a nucleotide sequence that encodes for a first fluorescent protein that fluoresces in a first color in the visible region when exposed to a wavelength of light that excites its chromophore, and the second heterologous polynucleotide further comprises a nucleotide sequence that encodes for a second fluorescent protein that fluoresces in a second color in the visible region when exposed to a wavelength of light that excites its chromophore.

35. A fusion protein comprising:
(A) a dimerizing peptide; and
(B) a mutant human enzyme dihydrofolate reductase (hDHFR) fragment comprising amino acid substitutions, numbered relative to SEQ ID NO: 3, of L22F, F31S, G2R, K54R, and T100I.

36. The fusion protein of claim 35, wherein the mutant hDHFR fragment further comprises an amino acid substitution, numbered relative to SEQ ID NO: 3, of L73I.

37. The fusion protein of claim 35, wherein the mutant hDHFR fragment further comprises amino acid substitutions, numbered relative to SEQ ID NO: 3, of S3P, R32K, S90A, R91K, and K98R.

38. The fusion protein of claim 35, wherein the dimerizing peptide comprises a GCN4 leucine zipper comprising SEQ ID NO: 254.

39. The fusion protein of claim 35, wherein the dimerizing peptide comprises the N7 hetero-dimerizing synthetic coiled coil peptide comprising SEQ ID NO: 255 or the N8 hetero-dimerizing synthetic coiled coil peptide comprising SEQ ID NO: 256, of the N7/N8 pair of hetero-dimerizing synthetic coiled coil peptides.

40. The fusion protein of claim 35, wherein the dimerizing peptide comprises the P7A hetero-dimerizing synthetic coiled coil peptide comprising SEQ ID NO: 257 or the P8A hetero-dimerizing synthetic coiled coil peptide comprising SEQ ID NO: 258, of the P7A/P8A pair of hetero-dimerizing synthetic coiled coil peptides.

41. The fusion protein of claim 35, wherein the first dimerization domain and the second dimerization domain each independently comprise an FKBP-based dimerization domain.

42. The fusion protein of claim 35, wherein the first dimerization domain and the second dimerization domain each independently comprise an FKBP mutant represented by SEQ ID NO: 192.

43. A nucleotide sequence coding for the fusion protein according to claim 35.

44. A transposon comprising the nucleotide sequence according to claim 43.

45. An isolated cell comprising the nucleotide sequence according to claim 43.

46. A method for producing the fusion protein according to claim 35, the method comprising culturing a cell comprising a nucleotide sequence coding for the fusion protein according to claim 35 under conditions conducive to the production of the fusion protein.

47. A composition comprising: (i) the fusion protein according to claim 35 or the nucleotide sequence according to claim 43; and (ii) a pharmaceutically acceptable carrier or excipient.

48-116. (canceled)

117. A method for determining rimiducid-dependent dimerization of FKBP proteins, the method comprising:
(A) co-transfecting a cell with:
(1) a first heterologous polynucleotide comprising a nucleotide sequence that encodes a first fusion protein, the first fusion protein comprising:
(a) a first engineered FKBP-based dimerization domain, wherein the first engineered FKBP-based dimerization domain has the ability to be activated upon binding of a chemical inducer of dimerization (CID); and
(b) a first dihydrofolate reductase (DHFR) fragment, and
(2) a second heterologous polynucleotide comprising a nucleotide sequence that encodes a second fusion protein, the second fusion protein comprising:
(a) a second engineered FKBP-based dimerization domain; and
(b) a second DHFR fragment that is normally contiguous to the first DHFR fragment,
wherein, the fragments of the DHFR protein sequence are catalytically inactive in isolation or when co-expressed in cells, but when brought into proximity with one another by fusion to protein domains that co-associate, demonstrate resistance to methotrexate; and
(B) subjecting the cell to:
(1) the CID; and
(2) methotrexate.

118. The method of claim 117, wherein the first DHFR fragment comprises amino acid substitutions, numbered relative to SEQ ID NO: 3, of L22F, F31S, G2R, K54R, and T100I.

119. The method of claim 118, wherein the second DHFR fragment comprises amino acid substitutions, numbered relative to SEQ ID NO: 3, of at least one of D168E and N185K.

120. The method of claim 119, wherein the first DHFR fragment further comprises an amino acid substitution, numbered relative to SEQ ID NO: 3, of L73I.

121. The method of claim 119, wherein the first DHFR fragment further comprises amino acid substitutions, numbered relative to SEQ ID NO: 3, of S3P, R32K, S90A, R91K, and K98R.

122. The method of claim 119, wherein the second DHFR fragment further comprises an amino acid substitution, numbered relative to SEQ ID NO: 3, of E154G.

123. The method of claim 122, wherein the second DHFR fragment further comprises amino acid substitutions, numbered relative to SEQ ID NO: 3, of K132R and D141E.

124. The method of claim 117, wherein the first DHFR fragment comprises amino acid substitutions, numbered relative to SEQ ID NO: 4, of L22F and F31S, and one or more of P3S, K32R, D69G, R84Q, A90S, K91R, and R98K.

125. The method of claim 124, wherein the second DHFR fragment comprises amino acid substitutions, numbered relative to SEQ ID NO: 4, of one or more of Q122K, Q127H, R132K, E141D, and G154E.

126. The method of claim 125, wherein the first DHFR fragment further comprises an amino acid substitution, numbered relative to SEQ ID NO: 4, of S107N.

127. The method of claim 125, wherein the second DHFR fragment further comprises an amino acid substitution, numbered relative to SEQ ID NO: 4, of S107N.

128. The method of claim 125, wherein the second DHFR fragment further comprises amino acid substitutions, numbered relative to SEQ ID NO: 4, of E168D or K185N, but not both.


SEQUENCE LISTING (192 sequences)

SEQ ID NO: 1 - DNA, 14 bp, synthetic construct
  misc_feature 1..14: note = Synthetic
  source 1..14: mol_type = other DNA
ccytttbmctgcca

SEQ ID NO: 2 - DNA, 14 bp, synthetic construct
  misc_feature 1..14: note = Synthetic
  source 1..14: mol_type = other DNA
tggcagkvaaargg

SEQ ID NO: 3 - protein, 187 aa, synthetic construct
  REGION 1..187: note = Synthetic
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 4 - protein, 187 aa, synthetic construct
  REGION 1..187: note = Synthetic
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 5 - DNA, 64 bp, synthetic construct
  misc_feature 1..64: note = Synthetic
  source 1..64: mol_type = other DNA
atcacgcatgggatacgtcgtggcagtaaaagggcttaaatgccaacgacgcgtcccatacgtt

SEQ ID NO: 6 - DNA, 82 bp, synthetic construct
  misc_feature 1..82: note = Synthetic
  source 1..82: mol_type = other DNA
atgacgcatgggatacgtcgtggcagtaaaagggcttaaatgccaacgacgcgtcccatacgttgttggcattttaagtctt

SEQ ID NO: 7 - DNA, 106 bp, synthetic construct
  misc_feature 1..106: note = Synthetic
  source 1..106: mol_type = other DNA
cctgggtaaactaaaagtcccctcgaggaaaggcccctaaagtgaaacagtgcaaaacgttcaaaaactgtctggcaatacaagttccactttgggacaaatcggc

SEQ ID NO: 8 - DNA, 105 bp, synthetic construct
  misc_feature 1..105: note = Synthetic
  source 1..105: mol_type = other DNA
cctgggtaaactaaaagtcccctcgaggaaaggcccctaaagtgaaacagtgcaaaacgttcaaaaactgtctggcaatacaagttccactttgaccaaaacggc

SEQ ID NO: 9 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHY

SEQ ID NO: 10 - protein, 589 aa, synthetic construct
  REGION 1..589: note = Synthetic
MAKRFYSAEEAAAHCMAPSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWNTTTVLSIPVFSATMSRNRYQLLLRFLHFNNNATAVPPDQPDHDRLHKLRPLIDSLSERFAAVYTPCQNICIDESLLLFKGRLRFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRTRKGLPRALLDKKLNRGETYALRKNELLAIKFFDKKNVFMLTSIHDESVIREQRVGRPPKNKPLCSKEYSKYMGGVDRTDQLQHYYNATRKTSAWYKKVGIYLIQMALRNSYIVYKAAVPGPKLSYYKYQLQILPALLFGGVEEQTVPEMLPSDNVARLIGKHFIDTLPPTPGKQRPQKGCKVCRKRGIRRDTRYYCPKCPRNPGLCFKPCFEIYHTQLHY

SEQ ID NO: 11 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNYEPINFFQLFMTEAILQDMVLYTNVYAEQYLTQNPLPRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHY

SEQ ID NO: 12 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQVPLPRYARAHAWHPTDIAEMKRFVGLTLAMGLIKANSLESYWDTTTVLNIPVFSATMSRNRYQLLLRFLEFNNEATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHY

SEQ ID NO: 13 - protein, 589 aa, synthetic construct
  REGION 1..589: note = Synthetic
MAKRFYSAEEAAAHCMASSQEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLLLFKGRLQFKQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGVYLIQMALRNSYIVYKAAVPGPKLSYYKYQLQILPALLFGGVEEQTVPEMPDSDNVARLIGKHFIDTLPPTPGKQRPQKGCKVCRKRGIRRDTRYYCPKCPRNPGLCRKPCFEIYHTQLHY

SEQ ID NO: 14 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDICEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLLLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGVYLIQMALRNSYIVYKAAVPGPKLSYYKYQLQILPALLFGGVEEQTVPEMPDSDNVARLIGKHFIDTLPPTPGKQRPQKGCKVCRKRGIRRDTRYYCPKCPRNPGLCRKPCFEIYHTQLHY

SEQ ID NO: 15 - protein, 589 aa, synthetic construct
  REGION 1..589: note = Synthetic
MAKRFYSAEEAAAHCMASSSEQTSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSIESYWDTTTVLSIPVFGATMSRNRYQLLLRFLHFNNNATAVPPDQPGHDRLHKLRPLIDSLSERFANVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRKPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 16 - protein, 589 aa, synthetic construct
  REGION 1..589: note = Synthetic
MAKRFYSAEEAAAHCSASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRGARAHAWHPTDIAEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 17 - protein, 589 aa, synthetic construct
  REGION 1..589: note = Synthetic
MAKRFYSAEEAAAHCSASSSEEFSGSDSEYVPPASESDSSTEESWCSSSTVSALEEPMEVDEDVDDLEDQEAHDRADAAAGGEPAWGPPCNFPPEIPPFTTVPGVKVDTSNFEPINFFQLFMTEAILQDMVLYTNVYAEQYLTQNPLTRGARAHAWHPTDIAEMKRFVGLTLAMGLIKANSIESYWDTTTVLSIPVFGATMSRNRYQLLHRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDSVARLIGKHFIDTLPPTPGKQRPQKGCKVCRKRGIRRDTRYYCPKCPRNPGLCRKPCFEIYHTQLHY

SEQ ID NO: 18 - protein, 589 aa, synthetic construct
  REGION 1..589: note = Synthetic
MAKRFYSAEEAAAHCSASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRGARAHAWHPTDIAEMKRFVGLTLAMGLIKANSIESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPDSDNVARLIGKHFIDTLPPTPGKQRPQKGCKVCRKRGIRRDTRYYCPKCPRNPGLCRKPCFEIYHTQLHY

SEQ ID NO: 19 - protein, 589 aa, synthetic construct
  REGION 1..589: note = Synthetic
MAKRFYSAEEAAAHCSASSSEQFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRGARAHAWYPTDIAEMKRFVGLTLAMGLIKANSIESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRGDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 20 - protein, 589 aa, synthetic construct
  REGION 1..589: note = Synthetic
MAKRFYSAEEAAAHCSASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRGARAHAWHPTDIAEMKRFVGLTLAMGLIKANSIESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYMSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRKPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDSVARLIGKHFIDTLPPTPGKQRPQKGCKVCRKRGIRRDTRYYCPKCPRNPGLCRKPCFEIYHTQLHY

SEQ ID NO: 21 - protein, 589 aa, synthetic construct
  REGION 1..589: note = Synthetic
MAKRFYSAEEAAAHCMASSSEEFYGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLLLFKGRLQFKQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPDSDNVARLIGKHFIDTLPPTPGKQRPQKGCKVCRKRGIRRDTRYYCPKCPRNPGLCRIPCFEIYHTQLHY

SEQ ID NO: 22 - protein, 589 aa, synthetic construct
  REGION 1..589: note = Synthetic
MAKRFYSAEEAAAHCSASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDICEMKRFVGLTLAMGLIKANSIESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYVLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 23 - protein, 589 aa, synthetic construct
  REGION 1..589: note = Synthetic
MAKRFYSAEEAAAHCSASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWYPTDIAEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 24 - protein, 589 aa, synthetic construct
  REGION 1..589: note = Synthetic
MAKRFYSAEEAAAHCSASSSDEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRGARAHAWYPTDIAEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLMLFKGRLQFRQYIPSKRARYGIKFYKLCESSTGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRKPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDSVARLIGKHFIDTLPPTPGKQRPQKGCKVCRKRGIRRDTRYYCPKCPRNPGLCRKPCFEIYHTQLHY

SEQ ID NO: 25 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFGATMSRNRYQLLLRFLHFNNNATAVPPDQPGHDRLHKLRPLIDSLSERFANVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 26 - protein, 589 aa, synthetic construct
  REGION 1..589: note = Synthetic
MAKRFYSAEEAAAHCMASSSEETSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRHGRPPKNKPLCSKEYSKYMGGVDRADQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 27 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFSATMSRNRYQLLLRFLHFNNEATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 28 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 29 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCRKPCFEIYHTQLHY

SEQ ID NO: 30 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWDTTTVLKIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLNTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRHWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHY

SEQ ID NO: 31 - protein, 589 aa, synthetic construct
  REGION 1..589: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQQLTQNPLPRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDRKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQYHY

SEQ ID NO: 32 - protein, 589 aa, synthetic construct
  REGION 1..589: note = Synthetic
MAKRFYSAEEAAAHCMASSSEEPSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTHLYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQYHY

SEQ ID NO: 33 - protein, 592 aa, synthetic construct
  REGION 1..592: note = Synthetic
MAKRFYSAEEAAAHCMASSQEEFSGSDSEYVPPASESDSSTEESWCSSSTVSALEEPMEVDEDVDDLEDQEAGDRVDAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKKNSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 34 - protein, 592 aa, synthetic construct
  REGION 1..592: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNKLPRYARAHAWHPTDIAEMKRFVGLTLAMGLIKANSLESYWDTGTVHSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 35 - protein, 592 aa, synthetic construct
  REGION 1..592: note = Synthetic
MAKRFCSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLTRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIATLPPTPGKQRPQKGCKVCRKRGIRRDTRYYCPKCPRNPGLCFKPCFEIYHTQLHYGRR

SEQ ID NO: 36 - protein, 592 aa, synthetic construct
  REGION 1..592: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQVPLTRYARAHAWHPTDIAEMKRFVGLTLAMGLIKKNSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 37 - protein, 592 aa, synthetic construct
  REGION 1..592: note = Synthetic
MAKRFYSAEEAAAHCMASSSEEFSGSDQEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNKLTRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 38 - protein, 592 aa, synthetic construct
  REGION 1..592: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNVLPRYARAHAWHPTDIAEMKRFVGLTLAMGLIKANSLESYWDTTTVLSIPVFSATMSRNRYQLLLRFLHFNNDATAVPPDQPGHDRLHKLRPLIDSLTERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 39 - protein, 592 aa, synthetic construct
  REGION 1..592: note = Synthetic
MAKRFYSAEEAAAHCMASSSEEFSGSDSEYVPPASESDSSTEESWCSSSTVSALEEPMEVDEDVDDLEDQEAGDRADAAPGGEPAWGPPCNFPPEIPPFTTVPGVKVDTSNFEPINFFQLFMTEAILQDMVLYTNVYAEQYLTQVPLPRYARAHAWHPTDIAEMKRFVGLTLAMGLIKANSLESYWDTTTVLSIPVFSATMSRNRYQLLLRFLHFNNEATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 40 - protein, 592 aa, synthetic construct
  REGION 1..592: note = Synthetic
MAKRFYSAEEAAAHCMASSSEEFSGSDSEYVPPASESDSSTEESWCSSSTVSALEEPMEVDEDVDDLEDQEAGDRVDAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWDTTTVLSIPVFSATMSRNRYQLLLKFLHFNNEATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 41 - protein, 592 aa, synthetic construct
  REGION 1..592: note = Synthetic
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQVPLPRYARAHAWHPTDIAEMKRFVGLTLAMGLIKANSLESYWDTTTVLNIPVFSATMSRNRYQLLLRFLEFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHYGRR

SEQ ID NO: 42 - DNA, 16 bp, synthetic construct
  misc_feature 1..16: note = Synthetic
  source 1..16: mol_type = other DNA
cccggcgagcatgagg

SEQ ID NO: 43 - DNA, 16 bp, synthetic construct
  misc_feature 1..16: note = Synthetic
  source 1..16: mol_type = other DNA
cctcatgctcgccggg

SEQ ID NO: 44 - DNA, 205 bp, synthetic construct
  misc_feature 1..205: note = Synthetic
  source 1..205: mol_type = other DNA
cagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtat

SEQ ID NO: 45 - DNA, 192 bp, synthetic construct
  misc_feature 1..192: note = Synthetic
  source 1..192: mol_type = other DNA
tttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttca

SEQ ID NO: 46 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYLAGLIKSNRQSLKDLWRTDGTGVDIFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 47 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYLAGLIKSNRQSLKDLWRTDGTGVDIFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFDVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELSANYNVSRNSKRWPMTLFYGVLNMAAINACIIYRANKNVTIKRTEFIRSLGLSMIYEHLHSRNKKKNIPTYLRQRIEKQLGEPSPRHVNVPGRYVRCQDCPYKKDRKTKHSCNACAKPICMEHAKFLCENCAELDSSL

SEQ ID NO: 48 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRWRQTKTAAENSSAETSFYMQETTLCELKALIGLLYIAGLIKSNRQSLKDLWRTDGTGVDIFRTTMSLQRFQFLLNNIRFDDKSTRDERKQTDNMAAFRSIFDQFVQSCQNAYSPSEFLTIDEMLLSFRGRCLFRVYIPNKPAKYGIKILALVDAKNFYVHNLEVYAGKQPSGPYAVSNRPFEVVERLIQPVARSHRNVTFDNWFTGYEVMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAHLDSSL

SEQ ID NO: 49 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRERASASRQVSGPFYTSKDGTKWYKNCQRPNVRLRSENIVTEQAQVKNIARDASTEYECWNIFVTSDMLQEILTHTNSSIRWRQTKTAAENSSAETSFYMQETTLCELKALIGLLYIAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELQANYNVSRNSKRWPMTLFYGVLNMAAINACIIYRTNKNVTIKRTEFIRSLGLSMIKEHLHSRNKKKNIPTYLRQRIEKQLGEPSPRHVNVPGRYVRCQDCPYKKDRKTKRSCNACAKPICMEHAKFLCENCAELDSSI

SEQ ID NO: 50 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRAVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYIAGLIKSNRQSLKDLWRTDGTGVDIFRTTMSLQRFQFLLNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRCNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 51 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQMSGPHYTSKDGTKWYKNCQRPNVRLRSENIVTEQAQVKNIARDASTEYECWNIFVTSDMLQEILTHTNSSIRWRQTKTAAENSSASTSFYMQETTLCELKALIGLLYIAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSHL

SEQ ID NO: 52 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRWRQTKTAAENSSASTSFYMQETTLCELKALIGLLYIAGLIKSNRQSLKDLWRTDGTGVDIFRTTMSLQRFQFLLNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIAMQLGEPSPRHVNVPGRYVRCQDCPYKKDRKTKRSCNACAKPICMEHAKFLCENCAELDSSL

SEQ ID NO: 53 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRWRQTKTAAENSSAETSFYMQETTLCELKALIGLLYIAGLIKSNRQSLKDLWRTDGTGVDIFRTTMSLQRFQFLLNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKTQIPENFIRTDRQPNSSVFGFQKDITLVSYAPKKNKVVVVMSTMHHDNSIDESTGEKQKPEMITFYNSTKAGVDVVDELQANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 54 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRWRQTKTAAENSSAETSFYMQETTLCELKALIGLLYLAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELQANYNVSRNSKRWPMTLFYGVLNMAAINACIIYRTNKNVTIKRTEFIRSLGLSMIKEHLHSRNKKKNIPTYLRQRIEKQLGEPSPRHVNYPGRYVRCQDCPYKKDRKTKRSCNACAKPICMEHAKFLCENCAELDSSL

SEQ ID NO: 55 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRWRQTKTAAENSSAETSFYMQETTLCELKALIGLLYIAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 56 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRWRQTKTAAENSSAETSFYMQETTLCELKALIGLLYLAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIKEHLHSRNKKKNIPTYLRQRIEKQLGEPSPRHVNYPGRYVRCQDCPYKKDRKTKRSCNACAKPICMEHAKFLCENCAELHSSL

SEQ ID NO: 57 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYLAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTSRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 58 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYIAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 59 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYIAGLIKSNRQSLKDLYRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELQANYNVSRNSKRWPMTLFYGVLNMAAINACIIYRTNKNVTIKRTEFIRSLGLSMIYEHLHSRNKKKNIPTYLRQRIEKQLGEPSPRHVNYPGRYVRCQDCPYKKDRKTKRSCNACAKPICMEHAKFLCENCAELDSSL

SEQ ID NO: 60 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYIAGLIKSNRQSLKDLWRKDGTGVDIFRTTMSLQRFQFLLNNIRFDDISTRDERKQTDNMAAFRSIFDQFVQCCQNAYSPSEFLTIDEMLLSFRGRCLFRVYIPNKPAKYGIKILALVDAKNFYVHNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKKWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 61 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYIAGLIKSNRQSLKDLWRTDGTGVDIFRTTMSLQRFQFLINNIRFDDKSTRDERKQTDNMAAFRSIFDQFVQCCQNAYSPSEFLTIDEMLLSFRGRCLFRVYIPNKPAKYGIKILALVDAKNWYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 62 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYLAGLIKSNRQSAKDLWRTDGTGVDIFRTTMSLQRFYFLQNNIRFDDKSTRDERKQTDNMAAFRSIFDQFVQCCQNAYSPSEFLTIDEMLLSFRGRCLFRVYIPNKPAKYGIKILALVDAKNFYVKNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 63 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLPELKALIALLYLAGLIKSNRQSLKDLWRTDGTGVDVFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFYVVNLEVYVGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 64 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRERQTKTAAENSSAETSFYMQETTLCELKALIALLYLAGLIKSNRQSLKDLWRTDGTGVDIFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFYVKNLEVYVGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 65 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYLAGLIKSNRQSLKDLWRTDGTGVDIFRTTMSLQRFQFLQNNIRFDDISTRDERKQTDNMAAFRSIFDQFVQCCQNAYSPSEFLTIDEMLLSFRGRCLFRVYIPNKPAKYGIKILALVDAKNDYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNYPGRYVRCQDCPYKKDRKTKRSCNACAKPICMEHAKFLCENCAELDSSL

SEQ ID NO: 66 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYLAGLIKSNRQSLKDLYRTDGTGVDIFRTTMSLQRFGFLQNNIRFDDKSTRDERKQTDNMAAFRSIFDQFVQCCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 67 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYLAGLIKSNRQSLKDLWRTDGTGVDIFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTILRQRIEKQLGEPSPRHVNVPGRYVRCQDCPYKKDRKTKRSCNACAKPICMEHAKFLCVNCAELDSSL

SEQ ID NO: 68 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIGLLYLAGLIKSNRQSLKDLWRTDGTGVDIFRTTMSLQRFQFLQNNIRFDDKSTRDERKQTDNMAAFRSIFDQFVQSCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNYPGRYVRCQDCPYKKDRKTKRSCNACAKPICMEHAKFLCENCAELDSSL

SEQ ID NO: 69 - protein, 610 aa, synthetic construct
  REGION 1..610: note = Synthetic
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYLAGLIKSNRQSLKDLWRTDGTGVDIFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYECMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEMQLGEPSPRHVNVPGRYVRCQDCPYKKDRKTKRSCNACAKPICMEHAKFLCENCAELKSSL

SEQ ID NO: 70 - DNA, 15 bp, synthetic construct
  misc_feature 1..15: note = Synthetic
  source 1..15: mol_type = other DNA
cacttggattgcggg

SEQ ID NO: 71 - DNA, 15 bp, synthetic construct
  misc_feature 1..15: note = Synthetic
  source 1..15: mol_type = other DNA
cccgacaccgtagtg

SEQ ID NO: 72 - DNA, 262 bp, synthetic construct
  misc_feature 1..262: note = Synthetic
  source 1..262: mol_type = other DNA
aaacgagttaagtcggctcgcgtgaattgcgcgtactccgcgggagccgtcttaactcggttcatatagatttgcggtggagtgcgggaaacgtgtaaactcgggccgattgtaactgcgtattaccaaatatttgtttccaagcttggtaccgagctcggatcccgtacgctgcaggtcgacggatccccgggttaattaaggcgcgccagatctgtttagcttgcctcgtccccgccgggtcacccggccagcgacatgg

SEQ ID NO: 73 - DNA, 227 bp, synthetic construct
  misc_feature 1..227: note = Synthetic
  source 1..227: mol_type = other DNA
tgtcgaagaattcggcggccgcatgcatctagagaattatttatgtactgaatagataaaaaaatgtctgtgattgaataaattttcattttttacacaagaaaccgaaaatttcatttcaatcgaacccatacttcaaaagatataggcattttaaactaactctgattttgcgcgggaaacctaaataattgcccgcgccatcttatattttggcgggaaattca

SEQ ID NO: 74 - protein, 567 aa, synthetic construct
  REGION 1..567: note = Synthetic
MSQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTWSPYTDSWYHIYMDNYYNSVANCEALMKNKFRICGTIRKNRGIPKDFQTISLKKGETKFIRKNDILLQVWQSKKPVYLISSHSAEMEESQNIDRTSKKKIVKPNALIDYNKHMKGVDRADQYLSYYSILRRWKWTKRLAMYMINCALFNSYAVYKSVRQRKMGFKMFLKQTAHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKN

SEQ ID NO: 75 - DNA, 13 bp, synthetic construct
  misc_feature 1..13: note = Synthetic
  source 1..13: mol_type = other DNA
ccctagaaagata

SEQ ID NO: 76 - DNA, 13 bp, synthetic construct
  misc_feature 1..13: note = Synthetic
  source 1..13: mol_type = other DNA
tatctttctaggg

SEQ ID NO: 77 - DNA, 296 bp, synthetic construct
  misc_feature 1..296: note = Synthetic
  source 1..296: mol_type = other DNA
gtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 78 - DNA, 218 bp, synthetic construct
  misc_feature 1..218: note = Synthetic
  source 1..218: mol_type = other DNA
tttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgat

SEQ ID NO: 79 - protein, 594 aa, synthetic construct
  REGION 1..594: note = Synthetic
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 80 - DNA, 18 bp, synthetic construct
  misc_feature 1..18: note = Synthetic
  source 1..18: mol_type = other DNA
cccttgrcatgcctggta

SEQ ID NO: 81 - DNA, 18 bp, synthetic construct
  misc_feature 1..18: note = Synthetic
  source 1..18: mol_type = other DNA
taccaggcatgycaaggg

SEQ ID NO: 82 - DNA, 297 bp, synthetic construct
  misc_feature 1..297: note = Synthetic
  source 1..297: mol_type = other DNA
gggtttattagacccaccactttgaaaaacctatgatatttttttaaattgaaggctattgttgacgtgtgttatagtagcttcgcgcaataaaccggcggccattttgacgagcgaacttcagtctcacgtgagcgtgcgtgcgagtagcacgtgtgtaaagtgcgcgcgggcccgtgggaccctaccaggcatacaacgtaacattctgtcggtaagaatattttctttattttttggcatttctttgtttaatgtgttaaattataatacgaaaaaaatattgttgcagtagaa

SEQ ID NO: 83 - DNA, 337 bp, synthetic construct
  misc_feature 1..337: note = Synthetic
  source 1..337: mol_type = other DNA
atcttttcgattatccaaagataatagtattttagttgatttattagtgccttaaattaatgaaagtctgacttcgatctctgcattatatgtaagattgttaattatagaactaagagtttaatttctgttaattaaaattaagcgattttgaataattgttaaataaagatattttcacatacatttacatattttatttattatctgtaataataatacattctaaaagacataaatataaaacaaaattttcctagcttgttcatttgtgtaaaacatgtattttcaatatcgggtttgacagacccaccaggcatgccgtgtgatttttatg

SEQ ID NO: 84 - protein, 588 aa, synthetic construct
  REGION 1..588: note = Synthetic
MARGLTDLEINQILELEDVENDVIFDESGDESDHVSIRVESDTEEVEIPTLEPQQGSSDSENDQPLSNLARRSFYKGKDNTIWNRAPPNPRVRTRSENIVTGTPGVKRQAKNALLELDCFHLFVNESILSVILEHTNHKIRSERQGKNTSNEYAYSETTLTELRAVIGLLYLAGLFKSGRQNLQDLWASDGTGIEIFPMTMSLRRFAFIVNCLRFDDSDTREERAAIDRLAPIRQIYEEFVKNCKDVYTPYENLTIDEELVAFRGRCKFRQYLPNKPAKYGIKIIALVDAYTYYSLNMEIYAGDQPDGPYKVSNKPHDVVDRIVQPISQTGRNVTMDNWFTSYPTYAHLLKNHKLTAVGTMKSNKTCIPPKFRERREINTSLFGFQDDFTIVSYIPKRNKNVFMLSSLHHDSEIDSETGEQQKPSIITFYNKTKSGVDNVDKLIRTYDVSRNSRRWPLTIFFWILNTAGINAKIVQMLNSSDNTPTRRAFIKKLGMSLIAPHQAERKTNSKIPVSLRKRIGSHLGESSASPAKIPNVGVKKRCYICPVKKDRKSKYICISCTSHICLEHANFVCENCRRNEEENSDSS

SEQ ID NO: 85 - DNA, 19 bp, synthetic construct
  misc_feature 1..19: note = Synthetic
  source 1..19: mol_type = other DNA
cctttarctrctgaggtgg

SEQ ID NO: 86 - DNA, 19 bp, synthetic construct
  misc_feature 1..19: note = Synthetic
  source 1..19: mol_type = other DNA
ccacctcagyagytaaagg

SEQ ID NO: 87 - DNA, 273 bp, synthetic construct
  misc_feature 1..273: note = Synthetic
  source 1..273: mol_type = other DNA
gggcttttcgagcctagcgaaagtgaaattgttcccctcctcccttcccccgcgcgcgacaaacccgtaacttctagtagcttcgatgttagttgcgcctaggccgtcagaagcttcgcacgtgttttcgtgcgcaattcggtaagtaaattcaatttgaaatttgtcgcgggcttcttaggccccacctcagtgtttacgtaacttttttgtaaatagtttcgattaagttattgtgttttttttttgcagtagcttgaaaacgtttgaaaa

SEQ ID NO: 88 - DNA, 370 bp, synthetic construct
  misc_feature 1..370: note = Synthetic
  source 1..370: mol_type = other DNA
ttttggtgcttgtattttttttcttcccataatacaaagataattatgaatgtgcctaatgctaaaaagactgttaaaaattaatattttatgtaagtttgttgattatttctaatattttaatgaatactttgtgatttttgatctcatgtgattttgccaaaaattttgctaagtgttttttaaaaacactcaaaagttaattataaataaaaaaattaaacaaaaaacattttattttatttaaaatctatccacaaaagcttattattatacaataaaacctaaaaaccccaaatattttaaaatatgaacatttatatacacgggccgcggaggcccccacgtcagtacttacgtgaaaataatt

SEQ ID NO: 89 - protein, 613 aa, synthetic construct
  REGION 1..613: note = Synthetic
MEPSTSSGRKRSIGNVHNQRAAKNRRAVVPGTRDFGTTLTSWLDNEDSSGSEVEDIGDNFTPERHEIESDTISQSESEEQVADHVTEEHNMSSDDDAPLSTRRSFYGKNRYKWACQPLSRAVRVPQHNIIQRTNVSNLTEDDPKDPFSIWNKLMDDEILQETLKWTNEKIIQYRSKFSDKDRPELRNLDMVELHAFIGLLLFTAVFKSNHENVNYLFATDGTGREIFRCVMSKNRFLVILHCLRFDNPDDREERRESDKIAAISYIFTKFVGNCQKIYNVCEYATVDEMLVPFRGRTHLMIYMPMKPAKYGLKLMCLCDANNGYFYNCYIYTGRGSDGAGLTEEEKKFMVPTQSVIHLAKPLFGSNRNITCDNWFTSIELIEYLKKKGLTCVGTMKKNKREIPKEFLPSKQRDVGSSLYGYAGQNTILSHVPKKNKAVILLSSMHHAEAVDETTGKPEIIGFYNKTKGGVDEIDKKCAIYTSSRRTRRWPMVVFYRMLDISTVNSHLIYDIHHDKTTERGMFLKQLARTLVLPQMKRRALNERLPRELRLSLARVLGPDMPVPDPQEVDETFKTRRRCHTCPLKLQRKSTHTCYTCKKHVCLQCAKQVCADCV

SEQ ID NO: 90 - DNA, 15 bp, synthetic construct
  misc_feature 1..15: note = Synthetic
  source 1..15: mol_type = other DNA
ccctcrtattatgtt

SEQ ID NO: 91 - DNA, 15 bp, synthetic construct
  misc_feature 1..15: note = Synthetic
  source 1..15: mol_type = other DNA
aacataataygaggg

SEQ ID NO: 92 - DNA, 15 bp, synthetic construct
  misc_feature 1..15: note = Synthetic
  source 1..15: mol_type = other DNA
ccctcatattatgtt

SEQ ID NO: 93 - DNA, 15 bp, synthetic construct
  misc_feature 1..15: note = Synthetic
  source 1..15: mol_type = other DNA
aacataatacgaggg

SEQ ID NO: 94 - DNA, 401 bp, synthetic construct
  misc_feature 1..401: note = Synthetic
  source 1..401: mol_type = other DNA
aagggtcaatttgacccatttcagtttttggtttgaccaaagaactggttatcctttctttttcttcacgaaagttggtgacttttcctcatctagggtcatgaacttgtgtgtaaaatctggatactgtgaagtgtcgtggaatgtctgtgaacagtttgtatacaaagatgatgttgcgggtcattttgacccacacactttgatgtgagcaagtagctgtccagatccgaaataaacatgtctctttgatgcactttattttgattgctaaattatttatattttgactgtctctgaatagaccttcagatcagagacccaggtgtgtgtgggggaggagctttctctcccttgtccttgtcactgttctcgtgtcatctctttgagaaacagcaaaa

SEQ ID NO: 95 - DNA, 247 bp, synthetic construct
  misc_feature 1..247: note = Synthetic
  source 1..247: mol_type = other DNA
agatactgaatattgaaaatctcagaaaatgtgacaagttaaattacaaaaaaaaagtgtttgtgaaggaaaaaaatattaaatatagtgttggaataaaaaaatagtattgtttgtctctttcctaaatgttgaaatattctaaaataaagttgatatcagtttaacctgtttttttattgttttgagtggatttacacagtatgggtcaaaatgacccgcaacataatcaaggtaattttttttc

SEQ ID NO: 96 - protein, 579 aa, synthetic construct
  REGION 1..579: note = Synthetic
MSSRRFTAEEALLLFFDSDAEEEISEIEDLSDAEDNDIDDPDFQFSDDEEDSEDESAVVSPSDENLGMEQSSSTEGTWASKDGNIKWSTSPHQSRGRLSSSNIIKMTPGPTRFAVTRVDDIQSAFQLFISQPIERIILDMTNLEGRRVFQEKWKSLDQTDLNAYIGILILAGVYRSKGEATSSLWNEENGRPIFRATMSLETFHMISRVIRFDNRDTRVGRRESDKLAAIRDVWDKWVEILPLLYNPGPHVTVDERLVPFRGRCPFRQYMPNKPAKYGIKIWAACDAKSSYAWKMQVYTGKSPGGAPEKNQGMRVVLEMSEGLQGHNITCDNFFTSYRLGEELQKRKLTMLGTVRRNKPELPSEILKIQGRPMHSSIFAFTEKATVVSYCPKRNKNVLVMSTMHTDASLSTRDDMKPQMILDYNSTKGGVDNLDKVTATYSCQRKTARWPMAIFFNIVDVSAYNAYVLWSEINQEWNAGKLYRRRLFLEELGKALITPKIQRRARPARSPAAAAVIEKIKFRTSNQFAMDPVDTDVKKRKRCQVCPSRDDSKTSTSCVKCKNFICRKHTVTFCPSCGEH

SEQ ID NO: 97 - DNA, 16 bp, synthetic construct
  misc_feature 1..16: note = Synthetic
  source 1..16: mol_type = other DNA
ccctagaagcccaatc

SEQ ID NO: 98 - DNA, 16 bp, synthetic construct
  misc_feature 1..16: note = Synthetic
  source 1..16: mol_type = other DNA
gattgggcttctaggg

SEQ ID NO: 99 - DNA, 303 bp, synthetic construct
  misc_feature 1..303: note = Synthetic
  source 1..303: mol_type = other DNA
tacgtaaatttgacgtataccgcggcgaaatatatctgtctctttcacgtttaccgtcggattcccgctaacttcggaaccaactcagtagccattgagaactcccaggacacagttgcgtcatctcggtaagtgccgccattttgttgtaatagacaggttgcacgtcattttgacgtataattgggctttgtgtaacttttgaaattatttataatttttattgatgtgatttatttgagttaatcgtattgtttcgttacatttttcatatgatattaatattttcagattgaatataaa

SEQ ID NO: 100 - DNA, 347 bp, synthetic construct
  misc_feature 1..347: note = Synthetic
  source 1..347: mol_type = other DNA
agactgttttttttaaaaggcttataaagtattactattgcgtgatttaattttataaaaatatttaaaaccagttgatttttttaataattacctaattttaagaaaaaatgttagaagcttgatatttttgttgatttttttctaagatttgattaaaaggccataattgtattaataaagagtatttttaacttcaaatttattttatttattaattaaaacttcaattatgataatacatgcaaaaatatagttcatcaacagaaaaatataggaaaactctaatagttttatttttacacgtcatttttacgtatgattgggctttatagctagtcaaatat

SEQ ID NO: 101 - protein, 598 aa, synthetic construct
  REGION 1..598: note = Synthetic
MESRQRLNQDEIATILENDDDYSPLDSDSEAEDRVVEDDVWSDNEDAMIDYVEDTSRQEDPDNNIASQESANLEVTSLTSHRIISLPQRSICGKNNHVWSTTKGRTTGRTSAINIIRTNRGPTRMCRNIVDPLLCFQLFITDEIIHEIVKWTNVEMIVKRQNLIDISASYRDTNTMEMWALVGILTLTAVMKDNHLSTDELFDATFSGTRYVSVMSRERFEFLIRCMRMDDKTLRPTLRSDDAFIPVRKLWEIFINQCRLNYVPGGNLTVDEQLLGFRGRCPFRMYIPNKPDKYGIRFPMMCDAATKYMIDAIPYLGKSTKTNGLPLGEFYVKELTKTVHGTNRNVTCDNWFTSIPLAKNMLQAPYNLTIVGTIRSNKREIPEEIKNSRSRPVGSSMFCFDGPLTLVSYKPKPSRMVFLLSSCDENAVINESNGKPDMILFYNQTKGGVDSFDQMCKSMSANRKTNRWPMAVFYGMLNMAFVNSYIIYCHNKINKQKKPINRKEFMKNLSTDLTTPWMQERLKAPTLKRTLRDNITNVLKNVVPPSPANNSEEPGPKKRSYCGFCSYKKRRMTKTQFYKCKKAICGEHNIDVCQDCVG

SEQ ID NO: 102 - DNA, 16 bp, synthetic construct
  misc_feature 1..16: note = Synthetic
  source 1..16: mol_type = other DNA
ccctagaagcccaatc

SEQ ID NO: 103 - DNA, 16 bp, synthetic construct
  misc_feature 1..16: note = Synthetic
  source 1..16: mol_type = other DNA
gattgggcttctaggg

SEQ ID NO: 104 - DNA, 304 bp, synthetic construct
  misc_feature 1..304: note = Synthetic
  source 1..304: mol_type = other DNA
tacgtaaatttgacgtataccgcggcgaaatatctctgttactttcacgtttaacgtcggatcgccgctaacttctgaaccaactcagtagccattgggacctcgcaggacacagttgcatcatctcggtaagtgccgccattttgttgtaatagagaggttgcacgtcattttgacgtataattgggctttgtgtaacttttgaaattgtttaaatttttttaaatttgtgatttatttgagttaatcgtattgtttcgttacattttacatgtaatattaatattttcaggttgaatacaaa

SEQ ID NO: 105 - DNA, 370 bp, synthetic construct
  misc_feature 1..370: note = Synthetic
  source 1..370: mol_type = other DNA
tgtttgtcaagactgtatataaagactgtttttttctaagaaacttataaaatattattacaagttgatttaattttatgaaaaaatttaaaactagttgattttttttataattacataattttaagaaaaagtgttagaggcttgatttttttgtttttttttttctaaggtttgattgaaatgccataatagtattaataaagagtattttttaacttaaaatctattttatttattaattaaaacttcaattatgataactcatgcaaaaatatagttcattaacagaaaaatcttggaaaactctgaagttttatttttacacgtcatttttacgtatgattgggctttataactagttaaatat

SEQ ID NO: 106 - protein, 598 aa, synthetic construct
  REGION 1..598: note = Synthetic
MASRQRLNHDEIATILENDDDYSPLDSESEKEDCVVEDDVWSDNEDAIVDFVEDTSAQEDPDNNIASRESPNLEVTSLTSHRIITLPQRSIRGKNNHVWSTTKGRTTGRTSAINIIRTNRGPTRMCRNIVDPLLCFQLFITDEIIHEIVKWTNVEIIVKRQNLKDISASYRDTNTMEIWALVGILTLTAVMKDNHLSTDELFDATFSGTRYVSVMSRERFEFLIRCIRMDDKTLRPTLRSDDAFLPVRKIWEIFINQCRQNHVPGSNLTVDEQLLGFRGRCPFRMYIPNKPDKYGIKFPMMCAAATKYMIDAIPYLGKSTKTNGLPLGEFYVKDLTKTVHGTNRNITCDNWFTSIPLAKNMLQAPYNLTIVGTIRSNKREMPEEIKNSRSRPVGSSMFCFDGPLTLVSYKPKPSKMVFLLSSCDENAVINESNGKPDMILFYNQTKGGVDSFDQMCKSMSANRKTNRWPMAVFYGMLNMAFVNSYIIYCHNKINKQEKPISRKEFMKKLSIQLTTPWMQERLQAPTLKRTLRDNITNVLKNVVPASSENISNEPEPKKRRYCGVCSYKKRRMTKAQCCKCKKAICGEHNIDVCQDCIG

SEQ ID NO: 107 - DNA, 32 bp, synthetic construct
  misc_feature 1..32: note = Synthetic
  source 1..32: mol_type = other DNA
cagttgaagtcggaagtttacatacacttaag

SEQ ID NO: 108 - DNA, 32 bp, synthetic construct
  misc_feature 1..32: note = Synthetic
  source 1..32: mol_type = other DNA
ctaaggtgtatgtaaacttccgacttcaactg

SEQ ID NO: 109 - DNA, 227 bp, synthetic construct
  misc_feature 1..227: note = Synthetic
  source 1..227: mol_type = other DNA
cagttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 110 - DNA, 229 bp, synthetic construct
  misc_feature 1..229: note = Synthetic
  source 1..229: mol_type = other DNA
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactg

SEQ ID NO: 111 - protein, 340 aa, synthetic construct
  REGION 1..340: note = Synthetic
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDKENYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 112 - DNA, 16 bp, synthetic construct
  misc_feature 1..16: note = Synthetic
  source 1..16: mol_type = other DNA
cagtgttcttcaacct

SEQ ID NO: 113 - DNA, 16 bp, synthetic construct
  misc_feature 1..16: note = Synthetic
  source 1..16: mol_type = other DNA
aggttgaagaacactg

SEQ ID NO: 114 - DNA, 328 bp, synthetic construct
  misc_feature 1..328: note = Synthetic
  source 1..328: mol_type = other DNA
cagtgttcttcaacctgtgttccgcggaaccctagggttccacccaaaggctttcggggttccgcgagtcattgcttcaattcgagagacgtcggccgcgccgctcttcagaatgcacatgcgtcaatcggagtttcatgttgaaacatgttatccattcgcatagttgacttacactgcacttaaccttaattttcaaaaatatgtaactgtacttgtggtcgtagttttgttgttgttttaggtttagacaagcaaaggtaagttaacttacagttttaaaataaattgtattttgtttgatcctaacctagaatcgttcagaaat

SEQ ID NO: 115 - DNA, 145 bp, synthetic construct
  misc_feature 1..145: note = Synthetic
  source 1..145: mol_type = other DNA
ccaaagcacgggctcaccttgttcgtaacaagtcaacgcagctgtccctaaaatctcatctgggtgtattactaaatgaagggttccataaaaaaaaatatctcgacaaagggttccgccggatggcaaaggttgaagaacactg

SEQ ID NO: 116 - protein, 636 aa, synthetic construct
  REGION 1..636: note = Synthetic
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 117 - protein, 163 aa, synthetic construct
  REGION 1..163: note = Synthetic
MNTEAARRSRARKLQRMKQLEDKVEELLSKNYHLENEVARLKKLVGERGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 118 - protein, 139 aa, synthetic construct
  REGION 1..139: note = Synthetic
MNTEAARRSRARKLQRMKQLEDKVEELLSKNYHLENEVARLKKLVGERGGGGSGGGGSASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 119 - protein, 147 aa, synthetic construct
  REGION 1..147: note = Synthetic
MGEIAALEAKNAALKAEIAALEAKIAALKAGYGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 120 - protein, 124 aa, synthetic construct
  REGION 1..124: note = Synthetic
MYGKIAALKAENAALEAKIAALKAEIAALEAGYGGGGSGGGGSASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 121 - protein, 147 aa, synthetic construct
  REGION 1..147: note = Synthetic
MGEIAALEAKNAALKAEIAALEAKNAALKAGCGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 122 - protein, 123 aa, synthetic construct
  REGION 1..123: note = Synthetic
MKIAALKAENAALEAKIAALKAENAALEAGGCGGGGSGGGGSASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 123 - protein, 130 aa, synthetic construct
  REGION 1..130: note = Synthetic
MKIAALKAENAALEAKIAALKAENAALEAGGCGGGGSGGGGSGGGGSLLASKVDMVWIVGGSSVYKEAMNHPGHLKLFVTRIMQDFESDTFFPEIDLEKYKLLPEYPGVLSDVQEEKGIKYKLEVYEKND

SEQ ID NO: 124 - protein, 130 aa, synthetic construct
  REGION 1..130: note = Synthetic
MKIAALKAENAALEAKIAALKAENAALEAGGCGGGGSGGGGSGGGGSLLASKVDMVWIVGGSSVYQEAMNHPGHLKLFVTRIMQDFESDTFFPEIDLEKYKLLPEYPGVLSDVQEEKGIKYKLEVYEKND

SEQ ID NO: 125 - protein, 130 aa, synthetic construct; identical to SEQ ID NO: 124
  REGION 1..130: note = Synthetic

SEQ ID NO: 126 - protein, 130 aa, synthetic construct; identical to SEQ ID NO: 123
  REGION 1..130: note = Synthetic

SEQ ID NO: 127 - protein, 130 aa, synthetic construct
  REGION 1..130: note = Synthetic
MKIAALKAENAALEAKIAALKAENAALEAGGCGGGGSGGGGSGGGGSLLASKVDMVWIVGGSSVYQEAMNQPGHLRLFVTRIMQEFESDTFFPEIDLEKYKLLPEYPGVLSDVQEEKGIKYKLEVYEKND

SEQ ID NO: 128 - protein, 130 aa, synthetic construct
  REGION 1..130: note = Synthetic
MKIAALKAENAALEAKIAALKAENAALEAGGCGGGGSGGGGSGGGGSLLASKVDMVWIVGGSSVYQEAMNQPGHLRLFVTRIMQEFESDTFFPEIDLGKYKLLPEYPGVLSDVQEEKGIKYKLEVYEKND

SEQ ID NO: 129 - protein, 130 aa, synthetic construct
  REGION 1..130: note = Synthetic
MKIAALKAENAALEAKIAALKAENAALEAGGCGGGGSGGGGSGGGGSLLASKVDMVWIVGGSSVYQEAMNQPGHLRLFVTRIMQEFESDTFFPEIDLGKYKLLPEYPGVLSEVQEEKGIKYKLEVYEKND

SEQ ID NO: 130 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYQEAMNQPGHLRLFVTRIMQEFESDTFFPEIDLGKYKLLPEYPGVLSEVQEEKGIKYKFEVYEKKD

SEQ ID NO: 131 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNQPGHLRLFVTRIMQEFESDTFFPEIDLGKYKLLPEYPGVLSEVQEEKGIKYKFEVYEKKD

SEQ ID NO: 132 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLRLFVTRIMQEFESDTFFPEIDLGKYKLLPEYPGVLSEVQEEKGIKYKFEVYEKKD

SEQ ID NO: 133 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQEFESDTFFPEIDLGKYKLLPEYPGVLSEVQEEKGIKYKFEVYEKKD

SEQ ID NO: 134 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLGKYKLLPEYPGVLSEVQEEKGIKYKFEVYEKKD

SEQ ID NO: 135 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLEKYKLLPEYPGVLSEVQEEKGIKYKFEVYEKKD

SEQ ID NO: 136 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLEKYKLLPEYPGVLSDVQEEKGIKYKFEVYEKKD

SEQ ID NO: 137 - protein, 130 aa, synthetic construct
  REGION 1..130: note = Synthetic
MKIAALKAENAALEAKIAALKAENAALEAGGCGGGGSGGGGSGGGGSLLASKVDMVWIVGGSSVYQEAMNQPGHLRLFVTRIMQEFESDTFFPEIDLGKYKLLPEYPGVLSEVQEEKGIKYKFEVYEKND

SEQ ID NO: 138 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLEKYKLLPEYPGVLSDVQEEKGIKYKLEVYEKKD

SEQ ID NO: 139 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLEKYKLLPEYPGVLSDVQEEKGIKYKFEVYEKND

SEQ ID NO: 140 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPEL

SEQ ID NO: 141 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESKYFQRMTTTSSVEGKQNLVIMGKKTWFSIPEKNRPLKGRINLVLSRELKEPPQGAHFLSRSLDDALKLTEQPEL

SEQ ID NO: 142 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGRKTWFSIPEKNRPLKGRINLVLSRELKEPPQGAHFLSRSLDDALKLTEQPEL

SEQ ID NO: 143 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKDRINLVLSRELKEPPQGAHFLSRSLDDALKLTEQPEL

SEQ ID NO: 144 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKGRINIVLSRELKEPPQGAHFLSRSLDDALKLTEQPEL

SEQ ID NO: 145 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKGRINLVLSRELKEPPRGAHFLSRSLDDALKLTEQPEL

SEQ ID NO: 146 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKGRINLVLSRELKEPPQGAHFLAKSLDDALKLTEQPEL

SEQ ID NO: 147 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALRLIEQPEL

SEQ ID NO: 148 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESKYFQRMTTTSSVEGKQNLVIMGRKTWFSIPEKNRPLKGRINLVLSRELKEPPQGAHFLSRSLDDALKLTEQPEL

SEQ ID NO: 149 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESKYFQRMTTTSSVEGKQNLVIMGRKTWFSIPEKNRPLKDRINIVLSRELKEPPQGAHFLSRSLDDALKLTEQPEL

SEQ ID NO: 150 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPQGAHFLSRSLDDALKLTEQPEL

SEQ ID NO: 151 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKGRINLVLSRELKEPPRGAHFLAKSLDDALRLIEQPEL

SEQ ID NO: 152 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKGRINIVLSRELKEPPRGAHFLAKSLDDALRLIEQPEL

SEQ ID NO: 153 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKDRINIVLSRELKEPPRGAHFLAKSLDDALRLIEQPEL

SEQ ID NO: 154 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGRKTWFSIPEKNRPLKDRINIVLSRELKEPPRGAHFLAKSLDDALRLIEQPEL

SEQ ID NO: 155 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 156 - protein, 152 aa, synthetic construct
  REGION 1..152: note = Synthetic
MGEIAALEAKNAALKAEIAALEAKNAALKAGCGGGGSGGGGSGGGGSVGPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 157 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLSRSLDDALKLTEQPEL

SEQ ID NO: 158 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLARSLDDALKLTEQPEL

SEQ ID NO: 159 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALKLTEQPEL

SEQ ID NO: 160 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLTEQPEL

SEQ ID NO: 161 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLTEQPEL

SEQ ID NO: 162 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGRKTWFSIPEKNRPLKDRINIVLSRELKEPPRGAHFLAKSLDDALKLTEQPEL

SEQ ID NO: 163 - protein, 152 aa, synthetic construct
  REGION 1..152: note = Synthetic
MGEIAALEAKNAALKAEIAALEAKNAALKAGCGGGGSGGGGSGGGGSVGSLNCIVAVSQNMGIGKNGDFPWPPLRNESRYFQRMTTTSSVEGKQNLVIMGKKTWFSIPEKNRPLKDRINIVLSRELKEPPRGAHFLSRSLDDALKLTEQPEL

SEQ ID NO: 164 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKGRINLVLSRELKEPPQGAHFLAKSLDDALRLIEQPEL

SEQ ID NO: 165 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGKKTWFSIPEKNRPLKGRINLVLSRELKEPPQGAHFLSKSLDDALRLIEQPEL

SEQ ID NO: 166 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALRLIEQPEL

SEQ ID NO: 167 - protein, 152 aa, synthetic construct
  REGION 1..152: note = Synthetic
MGEIAALEAKNAALKAEIAALEAKNAALKAGCGGGGSGGGGSGGGGSVRSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLIEQPEL

SEQ ID NO: 168 - protein, 152 aa, synthetic construct; identical to SEQ ID NO: 156
  REGION 1..152: note = Synthetic

SEQ ID NO: 169 - protein, 152 aa, synthetic construct
  REGION 1..152: note = Synthetic
MGEIAALEAKNAALKAEIAALEAKNAALKAGCGGGGSGGGGSGGGGSVRSLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 170 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESRYFQRMTTTSSVEGKQNLVIMGRKTWFSIPEKNRPLKDRINIVLSRELKEPPRGAHFLAKSLDDALRLIEQPEL

SEQ ID NO: 171 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGKKTWFSIPEKNRPLKDRINIVLSRELKEPPRGAHFLAKSLDDALRLIEQPEL

SEQ ID NO: 172 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKGRINIVLSRELKEPPRGAHFLAKSLDDALRLIEQPEL

SEQ ID NO: 173 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINLVLSRELKEPPRGAHFLAKSLDDALRLIEQPEL

SEQ ID NO: 174 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPQGAHFLAKSLDDALRLIEQPEL

SEQ ID NO: 175 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLSKSLDDALRLIEQPEL

SEQ ID NO: 176 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLARSLDDALRLIEQPEL

SEQ ID NO: 177 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALKLIEQPEL

SEQ ID NO: 178 - protein, 152 aa, synthetic construct; identical to SEQ ID NO: 160
  REGION 1..152: note = Synthetic

SEQ ID NO: 179 - protein, 152 aa, synthetic construct
  REGION 1..152: note = Synthetic
MGEIAALEAKNAALKAEIAALEAKNAALKAGCGGGGSGGGGSGGGGSVRSLNCIVAVSQNMGIGKNGDFPWPPLRNESRYFQRMTTTSSVEGKQNLVIMGRKTWFSIPEKNRPLKGRINLVLSRELKEPPQGAHFLARSLDDALKLIEQPEL

SEQ ID NO: 180 - protein, 152 aa, synthetic construct
  REGION 1..152: note = Synthetic
MGEIAALEAKNAALKAEIAALEAKNAALKAGCGGGGSGGGGSGGGGSVRSLNCIVAVSQNMGIGKNGDFPWPPLRNESRYFQRMTTTSSVEGKQNLVIMGRKTWFSIPEKNRPLKDRINLVLSRELKEPPQGAHFLARSLDDALKLIEQPEL

SEQ ID NO: 181 - protein, 152 aa, synthetic construct
  REGION 1..152: note = Synthetic
MGEIAALEAKNAALKAEIAALEAKNAALKAGCGGGGSGGGGSGGGGSVRPLNCIVAVSQNMGIGKNGDFPWPPLRNESKYFQRMTTTSSVEGKQNLVIMGRKTWFSIPEKNRPLKDRINLVLSRELKEPPQGAHFLAKSLDDALRLIEQPEL

SEQ ID NO: 182 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLGKYKLLPEYPGVLSDVQEEKGIKYKFEVYEKND

SEQ ID NO: 183 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLGKYKLLPEYPGVLSEVQEEKGIKYKFEVYEKND

SEQ ID NO: 184 - protein, 130 aa, synthetic construct
  REGION 1..130: note = Synthetic
MKIAALKAENAALEAKIAALKAENAALEAGGCGGGGSGGGGSGGGGSLLANKVDMVWIVGGSSVYKEAMNHPGHLKLFVTRIMQDFESDTFFPEIDLGKYKLLPEYPGVLSDVQEEKGIKYKFEVYEKKD

SEQ ID NO: 185 - protein, 48 aa, synthetic construct
  REGION 1..48: note = Synthetic
MNTEAARRSRARKLQRMKQLEDKVEELLSKNYHLENEVARLKKLVGER

SEQ ID NO: 186 - protein, 32 aa, synthetic construct
  REGION 1..32: note = Synthetic
MGEIAALEAKNAALKAEIAALEAKIAALKAGY

SEQ ID NO: 187 - protein, 33 aa, synthetic construct
  REGION 1..33: note = Synthetic
MYGKIAALKAENAALEAKIAALKAEIAALEAGY

SEQ ID NO: 188 - protein, 32 aa, synthetic construct
  REGION 1..32: note = Synthetic
MGEIAALEAKNAALKAEIAALEAKNAALKAGC

SEQ ID NO: 189 - protein, 32 aa, synthetic construct
  REGION 1..32: note = Synthetic
MKIAALKAENAALEAKIAALKAENAALEAGGC

SEQ ID NO: 190 - protein, 218 aa, synthetic construct
  REGION 1..218: note = Synthetic
MGVEIETISPGDGRTFPKKGQTCVVHYTGMLQNGKKVDSSRDRNKPFKFRIGKQEVIKGFEEGAAQMSLGQRAKLTCTPDVAYGATGHPGVIPPNATLIFDVELLNLESGGGSVRPLNCIVAVSQNMGIGKNGDFPWPPLRNESKYFQRMTTTSSVEGKQNLVIMGRKTWFSIPEKNRPLKDRINIVLSRELKEPPRGAHFLAKSLDDALRLIEQPEL

SEQ ID NO: 191 - protein, 194 aa, synthetic construct
  REGION 1..194: note = Synthetic
MGVEIETISPGDGRTFPKKGQTCVVHYTGMLQNGKKVDSSRDRNKPFKFRIGKQEVIKGFEEGAAQMSLGQRAKLTCTPDVAYGATGHPGVIPPNATLIFDVELLNLESGGGSASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 192 - protein, 108 aa, synthetic construct
  REGION 1..108: note = Synthetic
MGVEIETISPGDGRTFPKKGQTCVVHYTGMLQNGKKVDSSRDRNKPFKFRIGKQEVIKGFEEGAAQMSLGQRAKLTCTPDVAYGATGHPGVIPPNATLIFDVELLNLE